(19) United States
(12) Patent Application Publication
(10) Pub. No.: US 2024/0285379 A1
(43) Pub. Date: Aug. 29, 2024

(54) GRADUAL SURFACE QUALITY FEEDBACK DURING INTRAORAL SCANNING

(71) Applicant: Align Technology, Inc., San Jose, CA (US)

(72) Inventors: Ofer Saphier, Rechovot (IL); Shai Ayal, Tel Aviv (IL); Gilad Elbaz, Tel Aviv (IL); Pavel Gorodetsky, Rishon LeZion (IL); Eliran Dafna, Beer-sheva (IL); Ditza Auerbach, Aseret (IL); Michael Sabina, Campbell, CA (US); Shai Farkash, Hod Hasharon (IL)

(21) Appl. No.: 18/426,169

(22) Filed: Jan. 29, 2024

Related U.S. Application Data
(60) Provisional application No. 63/486,929, filed on Feb. 24, 2023.

(51) Int. Cl. A61C 9/00; A61B 1/00; A61B 1/24; A61C 13/34; G06T 7/00; G06T 19/00

(52) U.S. Cl. CPC A61C 9/006; A61B 1/00172; A61B 1/24; A61C 13/34; G06T 7/0012; G06T 19/00; G06T 2207/20081; G06T 2207/30036; G06T 2207/30168

(57) ABSTRACT

Embodiments relate to techniques for assisting users of intraoral scanners during intraoral scanning. In an embodiment, a method includes receiving intraoral scans of a dental site, generating a 3D surface of the dental site based on the intraoral scans, determining a surface quality score for a region of the 3D surface, and outputting a view of the 3D surface to a display, wherein the region of the 3D surface is shown with a visualization associated with the surface quality score.

### RELATED APPLICATIONS

The present application claims the benefit under 35 U.S.C. § 119(e) of U.S. Provisional Patent Application No. 63/486,929, filed Feb. 24, 2023, which is incorporated by reference herein.

### TECHNICAL FIELD

Embodiments of the present disclosure relate to the field of dentistry and, in particular, to a graphic user interface that provides visualizations during intraoral scanning.

### BACKGROUND

In prosthodontic procedures designed to implant a dental prosthesis in the oral cavity, the dental site at which the prosthesis is to be implanted in many cases should be measured accurately and studied carefully, so that a prosthesis such as a crown, denture or bridge, for example, can be properly designed and dimensioned to fit in place. A good fit enables mechanical stresses to be properly transmitted between the prosthesis and the jaw, and to prevent infection of the gums via the interface between the prosthesis and the dental site, for example.

Some procedures also call for removable prosthetics to be fabricated to replace one or more missing teeth, such as a partial or full denture, in which case the surface contours of the areas where the teeth are missing need to be reproduced accurately so that the resulting prosthetic fits over the edentulous region with even pressure on the soft tissues.

In some practices, the dental site is prepared by a dental practitioner, and a positive physical model of the dental site is constructed using known methods. Alternatively, the dental site may be scanned to provide 3D data of the dental site. In either case, the virtual or real model of the dental site is sent to the dental lab, which manufactures the prosthesis based on the model. However, if the model is deficient or undefined in certain areas, or if the preparation was not optimally configured for receiving the prosthesis, the design of the prosthesis may be less than optimal. For example, if the insertion path implied by the preparation for a closely-fitting coping would result in the prosthesis colliding with adjacent teeth, the coping geometry has to be altered to avoid the collision, which may result in the coping design being less optimal. Further, if the area of the preparation containing a finish line lacks definition, it may not be possible to properly determine the finish line and thus the lower edge of the coping may not be properly designed. Indeed, in some circumstances, the model is rejected and the dental practitioner then re-scans the dental site, or reworks the preparation, so that a suitable prosthesis may be produced.

In orthodontic procedures it can be important to provide a model of one or both jaws. Where such orthodontic procedures are designed virtually, a virtual model of the oral cavity is also beneficial. Such a virtual model may be obtained by scanning the oral cavity directly, or by producing a physical model of the dentition, and then scanning the model with a suitable scanner.

Thus, in both prosthodontic and orthodontic procedures, obtaining a three-dimensional (3D) model of a dental site in the oral cavity is an initial procedure that is performed. When the 3D model is a virtual model, the more complete and accurate the scans of the dental site are, the higher the quality of the virtual model, and thus the greater the ability to design an optimal prosthesis or orthodontic treatment appliance(s).

Some intraoral scanning systems provide two-state user feedback regarding the sufficiency of a 3D surface/3D model. The two-state feedback indicates either that the 3D surface is sufficient or that it is not sufficient. However, a user performing scanning does not know whether or not they are making progress in reaching the sufficient state and is left guessing as to whether or not he or she is making progress.

### SUMMARY

In a first implementation, a method comprises: receiving a plurality of intraoral scans of a dental site from an intraoral scanner during an intraoral scanning session; generating a three-dimensional (3D) surface of the dental site based on the plurality of intraoral scans; determining a first surface quality score for a first region of the 3D surface; outputting a first view of the 3D surface to a display, wherein the first region of the 3D surface is shown with a first visualization associated with the first surface quality score; receiving one or more additional intraoral scans of the dental site during the intraoral scanning session; updating the 3D surface based on the one or more additional intraoral scans; determining a new surface quality score for the first region of the updated 3D surface; and outputting a first view of the updated 3D surface to the display, wherein the first region of the updated 3D surface is shown with a second visualization associated with the new surface quality score.

A 2^{nd} implementation may further extend the 1^{st} implementation. In the 2^{nd} implementation, the method further comprises: inputting at least one of the first region of the 3D surface or the plurality of intraoral scans associated with the first region into a trained machine learning model, wherein the trained machine learning model outputs the first surface quality score.

A 3^{rd} implementation may further extend the 1^{st} or 2^{nd} implementation. In the 3^{rd} implementation, the plurality of intraoral scans are generated by projecting a structured light comprising a plurality of features onto the dental site and capturing the plurality of features on the dental site, and wherein the first surface quality score is determined based at least in part on a quantity of the plurality of features associated with the first region of the 3D surface.

A 4^{th} implementation may further extend the 3^{rd} implementation. In the 4^{th} implementation, the plurality of features comprise a plurality of spots.

A 5^{th} implementation, may further extend and of the 1^{st} through 4^{th} implementations. In the 5^{th} implementation, the 3D surface is generated based on a plurality of points from the plurality of intraoral scans, the method further comprising: determining a quality score for each point of the plurality of points; determining a subset of the plurality of points that are associated with the first region; and determining the first surface quality score for the first region based on quality scores of the subset of the plurality of points and on a quantity of the subset of the plurality of points.

A 6^{th} implementation may further extend the 5^{th} implementation. In the 6^{th} implementation, the quality score for a point of the plurality of points is computed based at least in part on a) a distance between the point and at least one of a first camera or a second camera of the intraoral scanner that captured the point in generation of an intraoral scan of the plurality of intraoral scans and b) a distance between the first camera and the second camera.

A 7^{th} implementation may further extend the 5^{th} or 6^{th} implementation. In the 7^{th} implementation, the quality score for a point of the plurality of points is computed based at least in part on a) a distance between the point and a camera of the intraoral scanner that captured the point in generation of an intraoral scan of the plurality of intraoral scans and b) a distance between the camera and a structured light projector of the intraoral scanner that projected structured light to the point.

An 8^{th} implementation may further extend any of the 5^{th} through 7^{th} implementations. In the 8^{th} implementation, the intraoral scanner comprises a plurality of cameras, and wherein the quality score for a point of the plurality of points is computed based at least in part on a number of the plurality of cameras that captured the point in generation of an intraoral scan of the plurality of intraoral scans.

An 9^{th} implementation may further extend any of the 5^{th} through 8^{th} implementations. In the 9^{th} implementation, a point of the plurality of points comprises a spot projected by a structured light projector of the intraoral scanner, and wherein the quality score for the point is based at least in part on a spot size of the spot.

A 10^{th} implementation may further extend any of the 5^{th} through 9^{th} implementations. In the 10^{th} implementation, the quality score for a point of the plurality of points is computed based at least in part on an angle between a normal to the 3D surface at the point and an imaging axis of the intraoral scanner.

An 11^{th} implementation may further extend any of the 5^{th} through 10^{th} implementations. In the 11^{th} implementation, the method further comprises: determining a type of material of the dental site at a point of the plurality of points, wherein the quality score for the point is computed based at least in part on the type of material of the dental site at the point.

A 12^{th} implementation may further extend any of the 5^{th} through 11^{th} implementations. In the 12^{th} implementation, the quality score for a point of the plurality of points is computed based on at least one of: a) a distance between the point and at least one of a first camera or a second camera of the intraoral scanner that captured the point in generation of an intraoral scan of the plurality of intraoral scans and b) a distance between the first camera and the second camera; a) a distance between the point and a camera of the intraoral scanner that captured the point in generation of the intraoral scan and b) a distance between the camera and a structured light projector of the intraoral scanner that projected structured light to the point; a number cameras of the intraoral scanner that captured the point in generation of the intraoral scan; a spot size associated with the point; an angle between a normal to the 3D surface at the point and an imaging axis of a camera that captured the point in generation the intraoral scan; or a type of material of the dental site at the point.

A 13^{th} implementation may further extend any of the 1^{st} through 12^{th} implementations. In the 13^{th} implementation, the first visualization comprises a first color and the second visualization comprises a second color.

A 14^{th} implementation may further extend any of the 1^{st} through 13^{th} implementations. In the 14^{th} implementation, the first visualization comprises a first transparency level and the second visualization comprises a second transparency level.

A 15^{th} implementation may further extend the 14^{th} implementation. In the 15^{th} implementation, the method further comprises: outputting a background to the display, wherein the background appears behind the 3D surface such that the background is visible through the first transparency level and the second transparency level.

A 16^{th} implementation may further extend the 14^{th} or 15^{th} implementations. In the 16^{th} implementation, the new surface quality score is greater than the first surface quality score, and wherein the second transparency level corresponding to the new surface quality score is lower than the first transparency level corresponding to the first surface quality score.

A 17^{th} implementation may further extend any of the 1^{st} through 16^{th} implementations. In the 17^{th} implementation, the first visualization comprises a first flicker rate and the second visualization comprises a second flicker rate.

An 18^{th} implementation may further the 17^{th} implementation. In the 18^{th} implementation, the first flicker rate and the second flicker rate comprise flickering of at least one of colors or transparency levels.

A 19^{th} implementation may further extend any of the 1^{st} through 18^{th} implementations. In the 19^{th} implementation, the first visualization comprises an arrow pointing to the first region and the second visualization comprises an updated arrow pointing to the first region.

A 20^{th} implementation may further extend the 19^{th} implementation. In the 20^{th} implementation, the new surface quality score is greater than the first surface quality score, and wherein the updated arrow comprises at least one of a shorter length, a lesser thickness or a lower flicker rate than the arrow.

A 21^{st} implementation may further extend any of the 1^{st} through 20^{th} implementations. In the 21^{st} implementation, the method further comprises: outputting a second view of the 3D surface to the display beside the first view, wherein the second view of the 3D surface lacks visualizations corresponding to surface quality scores

A 22^{nd} implementation may further extend the 21^{st} implementation. In the 22^{nd} implementation, the second view of the 3D surface comprises a color view showing captured colors of the dental site, a monochrome view, or a grayscale view.

A 23^{rd} implementation may further extend any of the 1^{st} through 22^{nd} implementations. In the 23^{rd} implementation, the method further comprises: determining a third surface quality score for a second region of the 3D surface; wherein the second region of the 3D surface is shown with a third visualization associated with the third surface quality score.

A 24^{th} implementation may further extend the 23^{rd} implementation. In the 24^{th} implementation, the method further comprises: determining a first grading rubric for the first region; determining the first visualization corresponding to the first surface quality score and the second visualization corresponding to the new surface quality score based on the first grading rubric; determining a second grading rubric for the second region; and determining the third visualization corresponding to the third surface quality score based on the second grading rubric.

A 25^{th} implementation may further extend the 24^{th} implementation. In the 25^{th} implementation, the method further comprises: classifying the first region as belonging to a first dental object class, wherein the first grading rubric is associated with the first dental object class; and classifying the second region as belonging to a second dental object class, wherein the second grading rubric is associated with the second dental object class.

A 26^{th} implementation may further extend the 25^{th} implementation. In the 26^{th} implementation, the first dental object class comprises a preparation tooth, an emergent profile, or a margin line, and wherein the second dental object class comprises a standard tooth.

A 27^{th} implementation may further extend any of the 4^{th} through 26^{th} implementations. In the 27^{th} implementation, the first grading rubric associates the first surface quality score with the first visualization, and wherein the second grading rubric associates a second surface quality score with the first visualization.

A 28^{th} implementation may further extend any of the 1^{st} through 27^{th} implementations. In the 28^{th} implementation, the method further comprises: determining whether a restorative treatment or an orthodontic treatment is to be performed for the dental site; and selecting a grading rubric that associates surface quality scores with visualizations based on whether the restorative treatment or the orthodontic treatment is selected.

A 29^{th} implementation may further extend any of the 1^{st} through 28^{th} implementations. In the 29^{th} implementation, the method further comprises: receiving a plurality of two-dimensional (2D) images of the dental site; and determining a quantity of the plurality of 2D images that depict the first region; wherein the first surface quality score for the first region is based at least in part on the quantity of the plurality of 2D images that depict the first region.

A 30^{th} implementation may further extend the 29^{th} implementation. In the 30^{th} implementation, the method further comprises: for each 2D image of the plurality of 2D images, determining an angle of a normal to the 3D surface at the first region to an imaging axis of the intraoral scanner; and determining a quantity of the plurality of 2D images for which the angle of the normal to the 3D surface at the first region to the imaging axis is within an angle threshold; wherein the first surface quality score for the first region is based at least in part on the quantity of the plurality of 2D images that depict the first region and that have angles of the normal to the 3D surface at the first region to the imaging axis that is within the angle threshold.

A 31^{st} implementation may further extend the 29^{th} or 30^{th} implementation. In the 31^{st} implementation, the plurality of 2D images comprise at least one of a plurality of color images or a plurality of near infrared (NIR) images.

A 32^{nd} implementation may further extend any of the 1^{st} through 31^{st} implementations. In the 32^{nd} implementation, the method further comprises: determining a first roughness and a first resolution associated with the first region of the 3D surface; wherein the first surface quality score is determined based at least in part on the first roughness and the first resolution.

A 33^{rd} implementation may further extend the 32^{nd} implementation. In the 33^{rd} implementation, the first resolution is determined based at least in part on a number of captured points in the first region; and the first roughness is determined based on distances between points from one or more intraoral scans of the plurality of intraoral scans used to generate the first region of the 3D surface and nearest points on the 3D surface.

A 34^{th} implementation may further extend the 33^{rd} implementation. In the 34^{th} implementation, the method further comprises: determining a standard deviation of distances between the points from the one or more intraoral scans and the nearest points on the 3D surface, wherein the first roughness is determined at least in part on the standard deviation.

A 35^{th} implementation may further extend the 34^{th} implementation. In the 35^{th} implementation, an increase in the standard deviation corresponds to an increase in the first roughness.

A 36^{th} implementation may further extend any of the 1^{st} through 35^{th} implementations. In the 36^{th} implementation, the method further comprises: determining that the new surface quality score for the first region has failed to improve for a threshold amount of time and that the new surface scanning quality score is below a surface quality threshold; determining one or more scanning suggestions that, if implemented, would cause the new surface quality score for the first region to improve; and outputting the one or more scanning suggestions to the display.

A 37^{th} implementation may further extend the 36^{th} implementation. In the 37^{th} implementation, the method further comprises: determining that the first region is associated with a distal molar of a patient being scanned, wherein the one or more scanning suggestions comprise a suggestion for a patient to move their jaw to the right or to the left.

A 38^{th} implementation may further extend the 36^{th} or 37^{th} implementation. In the 38^{th} implementation, the method further comprises: determining that the first region is at least partially obscured by soft tissue, wherein the one or more scanning suggestions comprise a suggestion for a doctor to roll the intraoral scanner about a prescribed axis and/or in a prescribed direction to move the soft tissue.

A 39^{th} implementation may further extend any of the 36^{th} through 38^{th} implementations. In the 39^{th} implementation, the method further comprises: determining that the first region is associated with an anterior tooth of a patient being scanned and that the first region is at least partially obscured by a patient lip, wherein the one or more scanning suggestions comprise guidance to at least one of a) pull the patient lip away from the anterior tooth or b) slide the intraoral scanner between the anterior tooth and the patient lip.

A 40^{th} implementation may further extend any of the 36^{th} through 39^{th} implementations. In the 40^{th} implementation, the one or more scanning suggestions comprise a path for the intraoral scanner to follow for capturing of further intraoral scans.

A 41^{st} implementation may further extend any of the 36^{th} through 40^{th} implementations. In the 41^{st} implementation, the one or more scanning suggestions comprise at least one of a target position or a target orientation to place the intraoral scanner for capturing of further intraoral scans.

A 42^{nd} implementation may further extend the 41^{st} implementation. In the 42^{nd} implementation, the method further comprises: receiving a first two-dimensional (2D) image of the dental site corresponding to a current field of view of the intraoral scanner at a first time; outputting the first 2D image to the display; and outputting a first overlay to the display over the first 2D image, the first overlay comprising a first shape approximately at a center of the 2D image and a second shape at the target position.

A 43^{rd} implementation may further extend the 42^{nd} implementation. In the 43^{rd} implementation, the method further comprises: receiving a second two-dimensional (2D) image of the dental site corresponding to the current field of view of the intraoral scanner at a second time after the intraoral scanner has been repositioned to move towards the target position; outputting the second 2D image to the display; and outputting a second overlay to the display over the second 2D image, the second overlay comprising the first shape approximately at the center of the 2D image and the second shape at the target position, wherein the first shape overlaps the second shape in the second overlay.

A 44^{th} implementation may further extend the 42^{nd} or 43^{rd} implementations. In the 44^{th} implementation, the first shape comprises a cross-hairs, a circle, a ring, or a square.

A 45^{th} implementation may further extend any of the 41^{st} through 44^{th} implementations. In the 45^{th} implementation, the method further comprises: outputting a first overlay to the display over the 3D surface, the first overlay comprising a shape at the target position and that indicates the target orientation.

A 46^{th} implementation may further extend any of the 36^{th} through 45^{th} implementations. In the 46^{th} implementation, the method further comprises: determining a current position of the intraoral scanner in a patient mouth, wherein the one or more scanning suggestions are determined based at least in part on the current position of the intraoral scanner in the patient mouth.

A 47^{th} implementation may further extend any of the 36^{th} through 46^{th} implementations. In the 47^{th} implementation, the one or more scanning suggestions comprise an animation showing how to move the intraoral scanner to achieve a target outcome.

A 48^{th} implementation may further extend any of the 36^{th} through 47^{th} implementations. In the 48^{th} implementation, the method further comprises: determining that the first region is at least partially obscured by a patient tongue, wherein the one or more scanning suggestions comprise a suggestion for a patient to move their tongue at least one of up, down, right, or left.

A 49^{th} implementation may further extend any of the 1^{st} through 48^{th} implementations. In the 49^{th} implementation, the method further comprises: determining that the new surface quality score for the first region has failed to improve for a threshold amount of time and that the new surface scanning quality score is below a surface quality threshold; and increasing a zoom setting for the first region.

A 50^{th} implementation may further extend the 49^{th} implementation. In the 50^{th} implementation, the method further comprises: determining that the intraoral scanner is focused on the first region prior to increasing the zoom setting for the first region.

A 51^{st} implementation may further extend any of the 1^{st} through 50^{th} implementations. In the 51^{st} implementation, the method further comprises: determining that the intraoral scanner has remained focused on the first region for a threshold amount of time; and increasing a zoom setting for the first region.

A 52^{nd} implementation may further extend the 51^{st} implementation. In the 52^{nd} implementation, the 3D surface comprises all of a dental arch that has been scanned thus far, wherein prior to increasing the zoom setting for the first region the first view of the 3D surface comprises an entirety of the 3D surface, and wherein after increasing the zoom setting for the first region the first view of the 3D surface comprises only a portion of the 3D surface.

A 53^{rd} implementation may further extend the 51^{st} or 52^{nd} implementation. In the 53^{rd} implementation, the first view of the 3D surface comprises at least one of a occlusal view, a birds-eye view, a distal to mesial view, or a mesial to distal view.

A 54^{th} implementation may further extend any of the 1^{st} through 53^{rd} implementations. In the 54^{th} implementation, the intraoral scanner comprises a plurality of cameras each having at least one of a different position or a different orientation in the intraoral scanner, the method further comprising: receiving a plurality of two-dimensional (2D) images each generated by a different camera of the plurality of cameras; determining a 2D image of the plurality of 2D images associated with improved intraoral scan quality; and outputting the determined 2D image to the display, wherein responsive to output of the determined 2D image, a doctor using the intraoral scanner will reposition the intraoral scanner in a manner that causes the improved intraoral scan quality.

A 55^{th} implementation may further extend any of the 1^{st} through 54^{th} implementations. In the 55^{th} implementation, the method further comprises: determining that the new surface quality score for the first region has failed to improve for a threshold amount of time; and making a determination that additional intraoral scans of the first region will not improve the surface quality score for the region.

A 56^{th} implementation may further extend the 55^{th} implementation. In the 56^{th} implementation, the method further comprises: determining that additional surface quality scores of one or more additional regions proximate to the first region at or above a surface quality threshold, wherein the determination is made responsive to determining that the additional surface quality scores of the one or more additional regions proximate to the first region at or above the surface quality threshold.

A 57^{th} implementation may further extend the 56^{th} implementation. In the 57^{th} implementation, the one or more additional regions comprise a plurality of additional regions that surround the first region.

A 58^{th} implementation may further extend any of the 55^{th} through 57^{th} implementations. In the 58^{th} implementation, the method further comprises: generating a notice to at least one of stop generating new intraoral scans of the first region or to move on to a next region.

A 59^{th} implementation may further extend any of the 55^{th} through 58^{th} implementations. In the 59^{th} implementation, the first region comprises at least one of the following region types: a hole having at least one of a bottom or one or more sidewalls that cannot be imaged by the intraoral scanner; a surface for which an achievable angle of the intraoral scanner relative to the hole is too steep to be imaged by the intraoral scanner; a surface covered by at least one of blood or saliva; or a surface covered by a collapsed gum.

A 60^{th} implementation may further extend the 59^{th} implementation. In the 60^{th} implementation, the method further comprises: determining a region type of the first region; and generating a notice identifying the region type.

A 61^{st} implementation may further extend any of the 55^{th} through 60^{th} implementations. In the 61^{st} implementation, at least one of a) determining that the new surface quality score for the first region has failed to improve for the threshold amount of time or b) making the determination that additional intraoral scans of the first region will not improve the surface quality score for the region are performed using a trained machine learning model.

A 62^{nd} implementation may further extend any of the 55^{th} through 61^{st} implementations. In the 62^{nd} implementation, the method further comprises: labeling the first region as a void on the updated 3D surface.

In a 63^{rd} implementation, a method comprises: receiving a plurality of intraoral scans of a dental site from an intraoral scanner during an intraoral scanning session; generating a three-dimensional (3D) surface of the dental site based on the plurality of intraoral scans; determining that a user of the intraoral scanner is having difficulty scanning a region of the dental site or had difficulty scanning the region of the dental site; and performing one or more actions to assist the user in scanning of the region of the dental site.

A 64^{th} implementation may further extend the 63^{rd} implementation. In the 64^{th} implementation, determining that the user is having difficulty scanning the region or had difficulty scanning the region of the dental site comprises: determining a surface quality score for a region of the 3D surface that corresponds to the region of the dental site; and determining that the surface quality score for the region of the 3D surface is below a surface quality threshold.

A 65^{th} implementation may further extend any of the 63^{rd} through 64^{th} implementations. In the 65^{th} implementation, determining that the user is having difficulty scanning the region comprises: determining that the intraoral scanner has remained focused on the region of the dental site without a threshold amount of improvement in a surface quality of a region of the 3D model that corresponds to the region of the dental site for a threshold amount of time.

A 66^{th} implementation may further extend any of the 63^{rd} through 65^{th} implementations. In the 66^{th} implementation, performing the one or more actions to assist the user in scanning of the dental site comprises: increasing a zoom setting for a region of the 3D surface that corresponds to the region of the dental site; and outputting a view of the 3D surface having the increased zoom setting to a display.

A 67^{th} implementation may further extend the 66^{th} implementation. In the 67^{th} implementation, the 3D surface comprises all of a dental arch that has been scanned thus far, wherein prior to increasing the zoom setting for the region the view of the 3D surface comprises an entirety of the 3D surface without the increased zoom setting, and wherein after increasing the zoom setting for the region of the 3D surface the view of the 3D surface comprises only a portion of the 3D surface.

A 68^{th} implementation may further extend any of the 66^{th} through 67^{th} implementations. In the 68^{th} implementation, the view of the 3D surface comprises at least one of a occlusal view, a birds-eye view, a distal to mesial view, or a mesial to distal view.

A 69^{th} implementation may further extend any of the 63^{rd} through 68^{th} implementations. In the 69^{th} implementation, performing the one or more actions to assist the user in scanning of the dental site comprises: determining one or more scanning suggestions that, if implemented, would cause a surface quality score for the region to improve; and outputting the one or more scanning suggestions to a display.

A 70^{th} implementation may further extend the 69^{th} implementation. In the 70^{th} implementation, the method further comprises: determining that the region is associated with a distal molar of a patient being scanned, wherein the one or more scanning suggestions comprise a suggestion for a patient to move their jaw at least one of to the right or to the left.

A 71^{st} implementation may further extend any of the 69^{th} through 70^{th} implementations. In the 71^{st} implementation, the method further comprises: determining that the region is at least partially obscured by soft tissue, wherein the one or more scanning suggestions comprise a suggestion for a doctor to roll the intraoral scanner about a prescribed axis and/or in a prescribed direction to move the soft tissue.

A 72^{nd} implementation may further extend any of the 69^{th} through 71^{st} implementations. In the 72^{nd} implementation, the method further comprises: determining that the region is associated with an anterior tooth of a patient being scanned and that the region is at least partially obscured by a patient lip, wherein the one or more scanning suggestions comprise guidance to at least one of a) pull the patient lip away from the anterior tooth or b) slide the intraoral scanner between the anterior tooth and the patient lip.

A 73^{rd} implementation may further extend any of the 69^{th} through 72^{nd} implementations. In the 73^{rd} implementation, the one or more scanning suggestions comprise a path for the intraoral scanner to follow for capturing of further intraoral scans.

A 74^{th} implementation may further extend any of the 69^{th} through 73^{rd} implementations. In the 74^{th} implementation, the one or more scanning suggestions comprise at least one of a target position or a target orientation to place the intraoral scanner for capturing of further intraoral scans.

A 75^{th} implementation may further extend the 74^{th} implementation. In the 75^{th} implementation, the method further comprises: receiving a first two-dimensional (2D) image of the dental site corresponding to a current field of view of the intraoral scanner at a first time; outputting the first 2D image to a display; and outputting a first overlay to the display over the first 2D image, the first overlay comprising a) a first shape at a first position of the 2D image that is associated with a center of a current field of view of the intraoral scanner and a b) second shape at the target position.

A 76^{th} implementation may further extend the 75^{th} implementation. In the 76^{th} implementation, the method further comprises: receiving a second two-dimensional (2D) image of the dental site corresponding to the current field of view of the intraoral scanner at a second time after the intraoral scanner has been repositioned to move towards the target position; outputting the second 2D image to the display; and outputting a second overlay to the display over the second 2D image, the second overlay comprising the first shape at the first position of the 2D image and the second shape at the target position, wherein the first shape overlaps the second shape in the second overlay.

A 77^{th} implementation may further extend the 75^{th} or 76^{th} implementation. In the 77^{th} implementation, the first shape comprises a cross-hairs, a circle, a ring, or a square.

A 78^{th} implementation may further extend any of the 74^{th} through 77^{th} implementations. In the 78^{th} implementation, the method further comprises: outputting a first overlay to the display over the 3D surface, the first overlay comprising a shape at the target position and that indicates the target orientation.

A 79^{th} implementation may further extend any of the 69^{th} through 78^{th} implementations. In the 79^{th} implementation, the method further comprises: determining a current position of the intraoral scanner in a patient mouth, wherein the one or more suggestions are determined based at least in part on the current position of the intraoral scanner in the patient mouth.

An 80^{th} implementation may further extend any of the 69^{th} through 79^{th} implementations. In the 80^{th} implementation, the one or more scanning suggestions comprise an animation showing how to move the intraoral scanner to achieve a target outcome.

An 81^{st} implementation may further extend any of the 69^{th} through 80^{th} implementations. In the 81^{st} implementation, the method further comprises: determining that the region is at least partially obscured by a patient tongue, wherein the one or more scanning suggestions comprise a suggestion for a patient to move their tongue at least one of up, down, right, or left.

An 82^{nd} implementation may further extend any of the 63^{rd} through 81^{st} implementations. In the 82^{nd} implementation, the method further comprises: determining that movement of the intraoral scanner has stopped at the region for at least a threshold amount of time before performing the one or more actions.

An 83^{rd} implementation may further extend any of the 63^{rd} through 82^{nd} implementations. In the 83^{rd} implementation, the method further comprises: determining that the intraoral scanner has begun moving away from the region before performing the one or more actions.

An 84^{th} implementation may further extend any of the 63^{rd} through 83^{rd} implementations. In the 84^{th} implementation, the method further comprises: performing the one or more actions responsive to receiving a request from a user of the intraoral scanner for scanning assistance.

An 85^{th} implementation may further extend any of the 63^{rd} through 84^{th} implementations. In the 85^{th} implementation, the method further comprises: receiving a set of intraoral images from the intraoral scanner, each intraoral image of the set of intraoral images having been generated by a different camera of the intraoral scanner; determining at least one of a current position or a current orientation of the intraoral scanner relative to the 3D surface; determining at least one of a target position or a target orientation of the intraoral scanner relative to the 3D surface; determining an image of the set of intraoral images that if selected would cause a user of the intraoral scanner to reposition the intraoral scanner in a manner that causes at least one of the current position or the current orientation of the intraoral scanner relative to the 3D surface to move towards at least one of the target position of the target orientation of the intraoral scanner relative to the 3D surface; selecting the determined image; and outputting the selected image to a display.

In an 86^{th} implementation, a method comprises: receiving at least one of a plurality of intraoral scans or a plurality of two-dimensional (2D) images of a dental site from an intraoral scanner; determining a velocity of the intraoral scanner relative to the dental site based at least in part on at least one of the plurality of intraoral scans or the plurality of 2D images; generating a three-dimensional (3D) surface of the dental site based on the plurality of intraoral scans; determining a zoom setting for displaying at least a portion of the 3D surface of the dental site based on the determined velocity; and outputting a view of at least the portion of the 3D surface, wherein at least the portion of the 3D surface has the determined zoom setting.

An 87^{th} implementation may further extend the 86^{th} implementation. In the 87^{th} implementation, the method further comprises: determining a current field of view of the intraoral scanner based on at least one of a most recent intraoral scan of the plurality of intraoral scans or a most recent 2D image of the plurality of 2D images; wherein the portion of the 3D surface corresponds to a portion of the dental site within the current field of view of the intraoral scanner.

An 88^{th} implementation may further extend any of the 86^{th} through 87^{th} implementations. In the 88^{th} implementation, the method further comprises: receiving at least one of a second plurality of intraoral scans or a second plurality of 2D images of the dental site from the intraoral scanner; determining a new velocity of the intraoral scanner relative to the dental site based at least in part on at least one of the second plurality of intraoral scans or the second plurality of 2D images; updating the 3D surface of the dental site based on the second plurality of intraoral scans; determining a new zoom setting for displaying at least the portion of the 3D surface of the dental site based on the new velocity; and outputting a view of at least the portion of the updated 3D surface, wherein at least the portion of the updated 3D surface has the new zoom setting.

An 89^{th} implementation may further extend any of the 86^{th} through 88^{th} implementations. In the 89^{th} implementation, the zoom setting is inversely proportional to the velocity.

A 90^{th} implementation may further extend any of the 86^{th} through 89^{th} implementations. In the 90^{th} implementation, the method further comprises: determining whether the velocity is below a velocity threshold; and responsive to determining that the velocity is below the velocity threshold, outputting a second view of at least the portion of the 3D surface beside the view of at least the portion of the 3D surface, wherein the portion of the 3D surface has a first orientation in the first view and a second orientation in the second view.

A 91^{st} implementation may further extend any of the 86^{th} through 90^{th} implementations. In the 91^{st} implementation, the method further comprises: determining a resolution to use for the portion of the 3D surface based on the velocity.

A 92^{nd} implementation may further extend any of the 86^{th} through 91^{st} implementations. In the 92^{nd} implementation, determining the velocity of the intraoral scanner relative to the dental site comprises determining an average velocity of the intraoral scanner relative to the dental site.

A 93^{rd} implementation may further extend the 92^{nd} implementation. In the 93^{rd} implementation, the average velocity is a weighted average that weights velocities computed from at least one of more recent intraoral scans or more recent 2D images more than velocities computed from at least one of less recent intraoral scans or less recent 2D images.

A 94^{th} implementation may further extend the 93^{rd} implementation. In the 94^{th} implementation, the average velocity is computed using an infinite impulse response filter.

A 95^{th} implementation may further extend any of the 86^{th} through 94^{th} implementations. In the 95^{th} implementation, determining the zoom setting based on the determined velocity is performed using a lookup table that maps velocity to zoom setting.

A 96^{th} implementation may further extend any of the 86^{th} through 95^{th} implementations. In the 96^{th} implementation, determining the zoom setting based on the determined velocity is performed using a function that maps velocity to zoom setting.

A 97^{th} implementation may further extend any of the 86^{th} through 96^{th} implementations. In the 97^{th} implementation, determining the velocity of the intraoral scanner comprises determining a velocity of a virtual point at a set distance from the intraoral scanner.

In a 98^{th} implementation, a method comprises: receiving a plurality of intraoral scans of a dental site from an intraoral scanner during an intraoral scanning session; determining a quality score for each point of a plurality of points in one or more of the plurality of intraoral scans; generating a three-dimensional (3D) surface based on the plurality of points from the one or more of the plurality of intraoral scans; determining a first surface quality score for a first region of the 3D surface based at least in part on a) a quantity of points, from the plurality of points, that are associated with the first region and b) quality scores of the points that are associated with the first region; and outputting a view of the 3D surface to a display, wherein the first region of the 3D surface is shown with a first visualization associated with the first surface quality score.

A 99^{th} implementation may further extend the 98^{th} implementation. In the 99^{th} implementation, the quality score for each point is determined before a depth value is determined for the point.

A 100^{th} implementation may further extend the 98^{th} or 99^{th} implementation. In the 100^{th} implementation, the quality score for each point is determined after a depth value is determined for the point.

A 101^{st} implementation may further extend any of the 98^{th} through 100^{th} implementations. In the 101^{st} implementation, the method further comprises: inputting the plurality of points from the one or more of the plurality of intraoral scans into a trained machine learning model, wherein the trained machine learning model outputs the quality score for each of the plurality of points.

A 102^{nd} implementation may further extend any of the 98^{th} through 101^{st} implementations. In the 102^{nd} implementation, the method further comprises: determining a first roughness and a first resolution associated with the first region of the 3D surface; wherein the first surface quality score is determined based at least in part on the first roughness and the first resolution.

A 103^{rd} implementation may further extend the 102^{nd} implementation. In the 103^{rd} implementation, the first resolution is determined based at least in part on the quantity of points that are associated with the first region; and the first roughness is determined based on distances between the points associated with the first region and nearest points on the 3D surface.

A 104^{th} implementation may further extend the 103^{rd} implementation. In the 104^{th} implementation, The method further comprises: determining a standard deviation of distances between the points associated with the first region and the nearest points on the 3D surface, wherein the first roughness is determined at least in part on the standard deviation.

A 105^{th} implementation may further extend the 104^{th} implementation. In the 105^{th} implementation, an increase in the standard deviation corresponds to an increase in the first roughness.

A 106^{th} implementation may further extend any of the 1^{st} through 105^{th} implementations. In the 98^{th} implementation, an intraoral scanning system comprises: the intraoral scanner; and a computing device, to perform the method of any of the 1^{st} through 105^{th} implementations.

A 107^{th} implementation may further extend any of the 1^{st} through 105^{th} implementations. In the 99^{th} implementation, a computer readable medium comprises instructions that, when executed by a processing device, cause the processing device to perform the method of any of the 1^{st} through 105^{th} implementations.

A 108^{th} implementation may further extend any of the 1^{st} through 105^{th} implementations. In the 100^{th} implementation, a computing device comprises a memory and one or more processor, wherein the one or more processor is to perform the method of any of the 1^{st} through 105^{th} implementations.

In a 109^{th} implementation, an intraoral scanning system comprises an intraoral scanner and a computing device, wherein the computing device is to: receive a plurality of intraoral scans of a dental site from the intraoral scanner during an intraoral scanning session; generate a three-dimensional (3D) surface of the dental site based on the plurality of intraoral scans; determine a first surface quality score for a first region of the 3D surface; receive one or more additional intraoral scans of the dental site during the intraoral scanning session; update the 3D surface based on the one or more additional intraoral scans; update the surface quality score for the first region based on the updated 3D surface; determine that the surface quality score for the first region fails to satisfy one or more criteria; and output a notice to at least one of stop generating new intraoral scans of the first region or to move on to a next region.

A 110^{th} implementation may extend the 109^{th} implementation. In the 110^{th} implementation to determine that the surface quality score for the first region fails to satisfy the one or more criteria the computing device is to: determine that the surface quality score for the first region has failed to improve for a threshold amount of time; and make a determination that additional intraoral scans of the first region will not improve the surface quality score for the region.

A 111^{th} implementation may extend the 109^{th} or 110^{th} implementations. In the 111^{th} implementation, to determine that the surface quality score for the first region fails to satisfy the one or more criteria the computing device is to: determine that the surface quality score for the first region is below a surface quality threshold and that that additional surface quality scores of one or more additional regions proximate to the first region are at or above the surface quality threshold.

### BRIEF DESCRIPTION OF THE DRAWINGS

Embodiments of the present disclosure are illustrated by way of example, and not by way of limitation, in the figures of the accompanying drawings.

FIG. 1 illustrates one embodiment of a system for performing intraoral scanning and/or generating a virtual three-dimensional model of an dental site.

FIG. 2A is a schematic illustration of a handheld intraoral scanner with a plurality cameras disposed within a probe at a distal end of the intraoral scanner, in accordance with some applications of the present disclosure.

FIGS. 2B-2C comprise schematic illustrations of positioning configurations for cameras and structured light projectors of an intraoral scanner, in accordance with some applications of the present disclosure.

FIG. 2D is a chart depicting a plurality of different configurations for the position of structured light projectors and cameras in a probe of an intraoral scanner, in accordance with some applications of the present disclosure.

FIG. 3 is a flow chart for a method of displaying a 3D surface of a dental site during intraoral scanning in a manner that shows surface quality, in accordance with embodiments of the present disclosure.

FIG. 4 is a flow chart for a method of displaying a 3D surface of a dental site during intraoral scanning in a manner that shows surface quality, in accordance with embodiments of the present disclosure.

FIG. 5 is a flow chart for a method of displaying a 3D surface of a dental site during intraoral scanning in a manner that shows different surface quality scores of different regions, in accordance with embodiments of the present disclosure.

FIG. 6 is a flow chart for a method of classifying regions of a 3D surface of a dental site and

determining surface quality scores associated with the regions using one or more trained machine learning model, in accordance with embodiments of the present disclosure.

FIG. 7 is a flow chart for a method of determining surface quality scores associated with a 3D surface of a dental site using a trained machine learning model, in accordance with embodiments of the present disclosure.

FIG. 8 is a flow chart for a method of determining surface quality scores associated with a 3D surface of a dental site, in accordance with embodiments of the present disclosure.

FIG. 9 illustrates scoring of regions of a dental site based on angle, in accordance with embodiments of the present disclosure.

FIG. 10 is a flow chart for a method of determining surface quality scores for 2D images, in accordance with embodiments of the present disclosure.

FIG. 11 is a flow chart for a method of determining surface quality scores for a region under consideration based on roughness and/or resolution, in accordance with embodiments of the present disclosure.

FIG. 12 illustrates scoring of regions of a dental site based on roughness and/or point density, in accordance with embodiments of the present disclosure.

FIGS. 13A-C illustrate views of a graphical user interface of an intraoral scan application that includes a 3D surface with surface quality feedback, in accordance with embodiments of the present disclosure.

FIG. 14A is a flow chart for a method of providing user assistance during intraoral scanning, in accordance with embodiments of the present disclosure.

FIG. 14B is a flow chart for a method of providing user assistance during intraoral scanning, in accordance with embodiments of the present disclosure.

FIG. 14C is a flow chart for a method of providing user assistance during intraoral scanning in instances where surface quality of a region is not improving, in accordance with embodiments of the present disclosure.

FIG. 15A is a flow chart for a method of adjusting a zoom setting to assist in intraoral scanning, in accordance with embodiments of the present disclosure.

FIG. 15B is a flow chart for a method of determining and outputting suggestions to aid in intraoral scanning during the intraoral scanning, in accordance with embodiments of the present disclosure.

FIG. 16A is a flow chart for a method of generating and outputting an overlay that provides scan guidance, in accordance with embodiments of the present disclosure.

FIG. 16B is a flow chart for a method of selecting an image (e.g., a viewfinder image) to influence how a dental site is scanned, in accordance with embodiments of the present disclosure.

FIGS. 17A-B illustrate sets of intraoral images generated by an array of cameras of an intraoral scanner, in accordance with embodiments of the present disclosure.

FIG. 18 illustrates reference frames of multiple cameras of an intraoral scanner, in accordance with an embodiment of the present disclosure.

FIG. 19 is a flow chart for a method of generating and outputting an overlay showing a suggested path to follow during intraoral scanning, in accordance with embodiments of the present disclosure.

FIG. 20 is a flow chart for a method of guiding a user of an intraoral scanner as to where to position the intraoral scanner during scanning, in accordance with embodiments of the present disclosure.

FIG. 21 illustrates a view of a 3D surface of a dental site being scanned, a position and orientation of a probe head of an intraoral scanner relative to the 3D surface, and a suggested next position and orientation of the probe head relative to the 3D surface, in accordance with an embodiment of the present disclosure.

FIGS. 22A-C illustrate a view of a 3D surface of a dental site being scanned and a suggested path for scanning the dental site, in accordance with an embodiment of the present disclosure.

FIG. 23 illustrates a view of a 3D surface of a dental site being scanned and a suggestion to rotate an intraoral scanner, in accordance with an embodiment of the present disclosure.

FIG. 24 illustrates a view of a 3D surface of a dental site being scanned and a suggestion for a patient to move their jaw, in accordance with an embodiment of the present disclosure.

FIG. 25 is a flow chart illustrating an embodiment for a method of determining scanning quality for regions of a 3D surface during intraoral scanning and outputting suggestions for improving the scanning quality.

FIG. 26 is a flow chart for a method of adjusting a zoom setting of a 3D surface based on scanner velocity, in accordance with embodiments of the present disclosure.

FIG. 27 is a flow chart for a method of adjusting a zoom setting of a 3D surface based on scanner velocity, in accordance with embodiments of the present disclosure.

FIG. 28A illustrates a view of a 3D surface of a dental site with a first zoom setting, in accordance with an embodiment of the present disclosure.

FIG. 28B illustrates a view of a 3D surface of a dental site with a second zoom setting, in accordance with an embodiment of the present disclosure.

FIG. 29 illustrates a block diagram of an example computing device, in accordance with embodiments of the present disclosure.

### DETAILED DESCRIPTION

Described herein are methods and systems for providing useful and gradual real time scanning feedback such as visualizations of intraoral objects (e.g., dental sites) reflective of scanning quality during intraoral scanning. Also described herein are methods and systems for aiding a user in intraoral scanning, such as by providing suggestions to improve scanning, by automatically adjusting zoom settings during intraoral scanning, by automatically selecting images for display during intraoral scanning, by automatically adjusting a resolution to use for a portion of a 3D surface during intraoral scanning, and so on. Examples of real time visualizations that may be provided in embodiments include representations of 3D surfaces of dental sites, representations of an intraoral scanner relative to the 3D surface(s), representations of suggested positions/orientations of the intraoral scanner relative to the 3D surfaces, representations of regions of 3D surfaces with visualizations representing quality scores associated with those regions, a representation of a recommended path for an intraoral scanner to follow for subsequent scanning, a representation of a target position and/or orientation of an intraoral scanner relative to a current position and/or orientation of the intraoral scanner, a representation of a void in a scanned surface, and so on.

In embodiments, an intraoral scan application can continuously update a 3D surface and adjust a view of the 3D surface and/or an intraoral scanner during intraoral scanning. As scanning progresses, more and more information is received for regions of the 3D surface. As further information is received for a region of the 3D surface, the 3D surface may be updated using the further information and a quality score associated with the region may be updated. The region of the 3D surface may be displayed with a visualization that is determined based on the current quality score associated with the region. Multiple different techniques may be used to determine the quality scores for regions of the 3D surface, for points on the 3D surface and/or for intraoral scans and/or images in embodiments. Accordingly, processing logic shows a user surface quality accumulation during scanning, so that the user will see gradual improvement in problematic areas, and can decide how much effort to apply in scanning those areas. In some instances, no improvement may be made in a problematic area. This may cause processing logic to determine that the problematic area cannot be successfully scanned, and to output a notice for a user to stop trying to scan the problematic area.

Intraoral scanners may have multiple surface capture challenges, such as a dental object having a reflective surface material that is difficult to capture, dental sites for which an angle of a surface of the dental site to an imaging axis is high (which makes that surface difficult to accurately capture), portions of dental sites that are far away from the intraoral scanner and thus have a higher noise and/or error, portions of dental sites that are too close to the intraoral scanner and have error, dental sites that are captured while the scanner is moving too quickly, resulting in blurry data and/or partial capture of an area, accumulation of blood and/or saliva over a dental site, and so on. Some or all of these challenges may cause a high level of noise in generated intraoral scans. Additionally, some captured areas of a dental site may have a sparse amount of captured points (e.g., a few number of points per mm²), resulting in a lower accuracy generated 3D surface. Embodiments address each of these challenges and enable a user to receive gradual and real time or near real time surface quality feedback that may take into account some or all of these challenges that a scanner might have encountered or might be presently encountering. In embodiments, even with a high level of noise, if a large number of points are captured for a region surface construction algorithms are able to estimate a 3D surface with a high degree of accuracy.

In embodiments, the surface quality scores for regions of a 3D surface of a dental site are continuously or periodically updated during intraoral scanning. This enables a user to determine whether they are making progress in the scanning process. By providing gradual feedback that is based on quality of a generated 3D surface (or regions thereof) during scanning, a user is able to determine which regions of the 3D surface are improving and which regions of the 3D surface are not improving. Different regions may be associated with different quality thresholds in embodiments. For example, higher quality thresholds may be associated with a preparation tooth than with other teeth. A user is able to see even minor improvements in the quality of a region of the 3D surface and can focus their attention and scanning on important regions (e.g., a preparation tooth) that require high quality during scanning, until a quality of those surfaces reaches a threshold quality.

In embodiments, a velocity of a scanner and/or virtual point (e.g., a focal point within a field of view of the scanner) is determined, and a zoom setting for displaying a generated 3D surface is controlled based on the velocity. As the velocity changes, the zoom setting for the 3D surface is automatically adjusted in embodiments. A smoothing operation may be used to smooth out adjustments in the zoom setting to avoid jerky changes in zoom settings.

In embodiments, processing logic assesses information received during intraoral scanning and determines whether a user of an intraoral scanner is having trouble scanning a region of a dental site based on the information. For example, processing logic may determine that a user is having trouble scanning a region if the intraoral scanner remains focused on the region for a threshold amount of time, if a quality score for the region is not improving, if a velocity of the intraoral scanner and/or a point of focus of the intraoral scanner is below a threshold, and so on. Responsive to determining that the user is having trouble scanning the region, the processing logic may determine one or more actions that may improve scan quality and/or may provide one or more suggestions for improving the scan quality. Many different actions may be performed and/or many different suggestions may be provided, examples of which are provided hereinbelow. Alternatively, the processing logic may determine that improved scan quality for the region is unobtainable, and may output a suggestion to stop scanning the region and/or to proceed with scanning a next region.

Various embodiments are described herein. It should be understood that these various embodiments may be implemented as stand-alone solutions and/or may be combined. Accordingly, references to an embodiment, or one embodiment, may refer to the same embodiment and/or to different embodiments. Some embodiments are discussed herein with reference to intraoral scans and intraoral images. However, it should be understood that embodiments described with reference to intraoral scans also apply to lab scans or model/impression scans. A lab scan or model/impression scan may include one or more images of a dental site or of a model or impression of a dental site, which may or may not include height maps, and which may or may not include intraoral two-dimensional (2D) images (e.g., 2D color images).

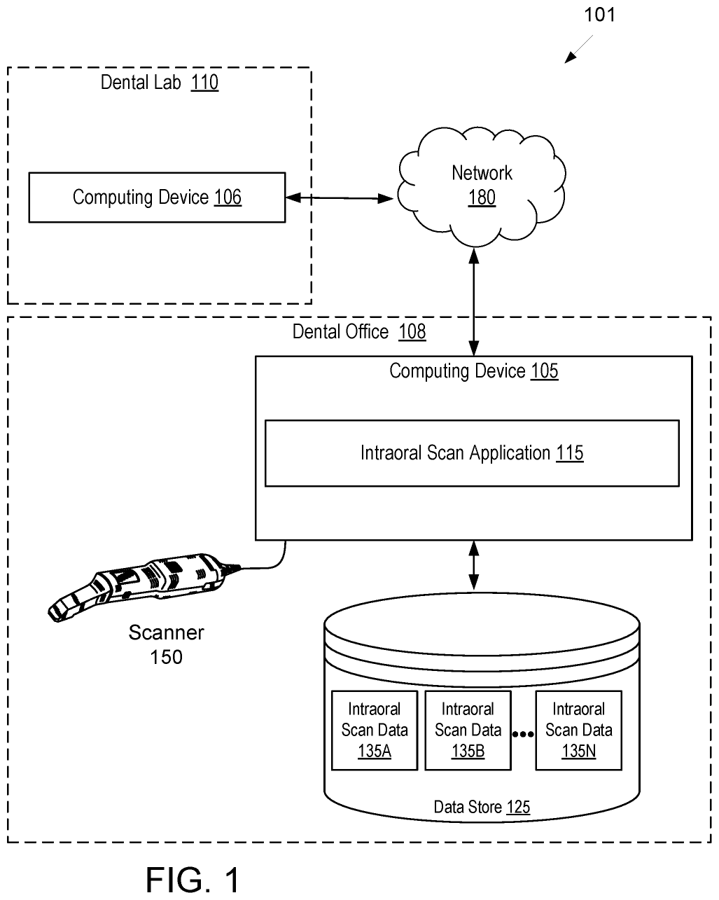

FIG. 1 illustrates one embodiment of a system 101 for performing intraoral scanning and/or generating a three-dimensional (3D) surface and/or a virtual three-dimensional model of a dental site. System 101 includes a dental office 108 and optionally one or more dental lab 110. The dental office 108 and the dental lab 110 each include a computing device 105, 106, where the computing devices 105, 106 may be connected to one another via a network 180. The network 180 may be a local area network (LAN), a public wide area network (WAN) (e.g., the Internet), a private WAN (e.g., an intranet), or a combination thereof.

Computing device 105 may be coupled to one or more intraoral scanner 150 (also referred to as a scanner) and/or a data store 125 via a wired or wireless connection. In one embodiment, multiple scanners 150 in dental office 108 wirelessly connect to computing device 105. In one embodiment, scanner 150 is wirelessly connected to computing device 105 via a direct wireless connection. In one embodiment, scanner 150 is wirelessly connected to computing device 105 via a wireless network. In one embodiment, the wireless network is a Wi-Fi network. In one embodiment, the wireless network is a Bluetooth network, a Zigbee network, or some other wireless network. In one embodiment, the wireless network is a wireless mesh network, examples of which include a Wi-Fi mesh network, a Zigbee mesh network, and so on. In an example, computing device 105 may be physically connected to one or more wireless access points and/or wireless routers (e.g., Wi-Fi access points/routers). Intraoral scanner 150 may include a wireless module such as a Wi-Fi module, and via the wireless module may join the wireless network via the wireless access point/router.

Computing device 106 may also be connected to a data store (not shown). The data stores may be local data stores and/or remote data stores. Computing device 105 and computing device 106 may each include one or more processing devices, memory, secondary storage, one or more input devices (e.g., such as a keyboard, mouse, tablet, touchscreen, microphone, camera, and so on), one or more output devices (e.g., a display, printer, touchscreen, speakers, etc.), and/or other hardware components.

In embodiments, scanner 150 includes an inertial measurement unit (IMU). The IMU may include an accelerometer, a gyroscope, a magnetometer, a pressure sensor and/or other sensor. For example, scanner 150 may include one or more micro-electromechanical system (MEMS) IMU. The IMU may generate inertial measurement data (also referred to as movement data), including acceleration data, rotation data, and so on.

Computing device 105 and/or data store 125 may be located at dental office 108 (as shown), at dental lab 110, or at one or more other locations such as a server farm that provides a cloud computing service. Computing device 105 and/or data store 125 may connect to components that are at a same or a different location from computing device 105 (e.g., components at a second location that is remote from the dental office 108, such as a server farm that provides a cloud computing service). For example, computing device 105 may be connected to a remote server, where some operations of intraoral scan application 115 are performed on computing device 105 and some operations of intraoral scan application 115 are performed on the remote server.

Some additional computing devices may be physically connected to the computing device 105 via a wired connection. Some additional computing devices may be wirelessly connected to computing device 105 via a wireless connection, which may be a direct wireless connection or a wireless connection via a wireless network. In embodiments, one or more additional computing devices may be mobile computing devices such as laptops, notebook computers, tablet computers, mobile phones, portable game consoles, and so on. In embodiments, one or more additional computing devices may be traditionally stationary computing devices, such as desktop computers, set top boxes, game consoles, and so on. The additional computing devices may act as thin clients to the computing device 105. In one embodiment, the additional computing devices access computing device 105 using remote desktop protocol (RDP). In one embodiment, the additional computing devices access computing device 105 using virtual network control (VNC). Some additional computing devices may be passive clients that do not have control over computing device 105 and that receive a visualization of a user interface of intraoral scan application 115. In one embodiment, one or more additional computing devices may operate in a master mode and computing device 105 may operate in a slave mode.

Intraoral scanner 150 may include a probe (e.g., a hand held probe) for optically capturing three-dimensional structures. The intraoral scanner 150 may be used to perform an intraoral scan of a patient's oral cavity. An intraoral scan application 115 running on computing device 105 may communicate with the scanner 150 to effectuate the intraoral scan. A result of the intraoral scan may be intraoral scan data 135A, 135B through 135N that may include one or more sets of intraoral scans and/or sets of intraoral 2D images. Each intraoral scan may include a 3D image or point cloud that may include depth information (e.g., a height map) of a portion of a dental site. In embodiments, intraoral scans include x, y and z information.

Intraoral scan data 135A-N may also include color 2D images and/or images of particular wavelengths (e.g., near-infrared (NIRI) images, infrared images, ultraviolet images, etc.) of a dental site in embodiments. In embodiments, intraoral scanner 150 alternates between generation of 3D intraoral scans and one or more types of 2D intraoral images (e.g., color images, NIRI images, etc.) during scanning. For example, one or more 2D color images may be generated between generation of a fourth and fifth intraoral scan by outputting white light and capturing reflections of the white light using multiple cameras.

Intraoral scanner 150 may include multiple different cameras (e.g., each of which may include one or more image sensors) that generate 2D images (e.g., 2D color images) of different regions of a patient's dental arch concurrently. These 2D images may be stitched together to form a single 2D image representation of a larger field of view that includes a combination of the fields of view of the multiple cameras. Intraoral 2D images may include 2D color images, 2D infrared or near-infrared (NIRI) images, and/or 2D images generated under other specific lighting conditions (e.g., 2D ultraviolet images). The 2D images may be used by a user of the intraoral scanner to determine where the scanning face of the intraoral scanner is directed and/or to determine other information about a dental site being scanned.

The scanner 150 may transmit the intraoral scan data 135A, 135B through 135N to the computing device 105. Computing device 105 may store the intraoral scan data 135A-135N in data store 125.

According to an example, a user (e.g., a practitioner) may subject a patient to intraoral scanning. In doing so, the user may apply scanner 150 to one or more patient intraoral locations. The scanning may be divided into one or more segments (also referred to as roles). As an example, the segments may include a lower dental arch of the patient, an upper dental arch of the patient, one or more preparation teeth of the patient (e.g., teeth of the patient to which a dental device such as a crown or other dental prosthetic will be applied), one or more teeth which are contacts of preparation teeth (e.g., teeth not themselves subject to a dental device but which are located next to one or more such teeth or which interface with one or more such teeth upon mouth closure), and/or patient bite (e.g., scanning performed with closure of the patient's mouth with the scan being directed towards an interface area of the patient's upper and lower teeth). Via such scanner application, the scanner 150 may provide intraoral scan data 135A-N to computing device 105. The intraoral scan data 135A-N may be provided in the form of intraoral scan data sets, each of which may include 2D intraoral images (e.g., color 2D images) and/or 3D intraoral scans of particular teeth and/or regions of an dental site. In one embodiment, separate intraoral scan data sets are created for the maxillary arch, for the mandibular arch, for a patient bite, and/or for each preparation tooth. Alternatively, a single large intraoral scan data set is generated (e.g., for a mandibular and/or maxillary arch). Intraoral scans may be provided from the scanner 150 to the computing device 105 in the form of one or more points (e.g., one or more pixels and/or groups of pixels). For instance, the scanner 150 may provide an intraoral scan as one or more point clouds. The intraoral scans may each comprise height information (e.g., a height map that indicates a depth for each pixel).

The manner in which the oral cavity of a patient is to be scanned may depend on the procedure to be applied thereto. For example, if an upper or lower denture is to be created, then a full scan of the mandibular or maxillary edentulous arches may be performed. In contrast, if a bridge is to be created, then just a portion of a total arch may be scanned which includes an edentulous region, the neighboring preparation teeth (e.g., abutment teeth) and the opposing arch and dentition. Alternatively, full scans of upper and/or lower dental arches may be performed if a bridge is to be created.

By way of non-limiting example, dental procedures may be broadly divided into prosthodontic (restorative) and orthodontic procedures, and then further subdivided into specific forms of these procedures. Additionally, dental procedures may include identification and treatment of gum disease, sleep apnea, and intraoral conditions. The term prosthodontic procedure refers, inter alia, to any procedure involving the oral cavity and directed to the design, manufacture or installation of a dental prosthesis at a dental site within the oral cavity (dental site), or a real or virtual model thereof, or directed to the design and preparation of the dental site to receive such a prosthesis. A prosthesis may include any restoration such as crowns, veneers, inlays, onlays, implants and bridges, for example, and any other artificial partial or complete denture. The term orthodontic procedure refers, inter alia, to any procedure involving the oral cavity and directed to the design, manufacture or installation of orthodontic elements at a dental site within the oral cavity, or a real or virtual model thereof, or directed to the design and preparation of the dental site to receive such orthodontic elements. These elements may be appliances including but not limited to brackets and wires, retainers, clear aligners, or functional appliances.

In embodiments, intraoral scanning may be performed on a patient's oral cavity during a visitation of dental office 108. The intraoral scanning may be performed, for example, as part of a semi-annual or annual dental health checkup. The intraoral scanning may also be performed before, during and/or after one or more dental treatments, such as orthodontic treatment and/or prosthodontic treatment. The intraoral scanning may be a full or partial scan of the upper and/or lower dental arches, and may be performed in order to gather information for performing dental diagnostics, to generate a treatment plan, to determine progress of a treatment plan, and/or for other purposes. The dental information (intraoral scan data 135A-N) generated from the intraoral scanning may include 3D scan data, 2D color images, NIRI and/or infrared images, and/or ultraviolet images, of all or a portion of the upper jaw and/or lower jaw. The intraoral scan data 135A-N may further include one or more intraoral scans showing a relationship of the upper dental arch to the lower dental arch. These intraoral scans may be usable to determine a patient bite and/or to determine occlusal contact information for the patient. The patient bite may include determined relationships between teeth in the upper dental arch and teeth in the lower dental arch.

For many prosthodontic procedures (e.g., to create a crown, bridge, veneer, etc.), an existing tooth of a patient is ground down to a stump. The ground tooth is referred to herein as a preparation tooth, or simply a preparation. The preparation tooth has a margin line (also referred to as a finish line), which is a border between a natural (unground) portion of the preparation tooth and the prepared (ground) portion of the preparation tooth. The preparation tooth is typically created so that a crown or other prosthesis can be mounted or seated on the preparation tooth. In many instances, the margin line of the preparation tooth is sub-gingival (below the gum line).

Intraoral scanners may work by moving the scanner 150 inside a patient's mouth to capture all viewpoints of one or more tooth. During scanning, the scanner 150 is calculating distances to solid surfaces in some embodiments. These distances may be recorded as images called ‘height maps’ or as point clouds in some embodiments. Each scan (e.g., optionally height map or point cloud) is overlapped algorithmically, or ‘stitched’, with the previous set of scans to generate a growing 3D surface. As such, each scan is associated with a rotation in space, or a projection, to how it fits into the 3D surface.

During intraoral scanning, intraoral scan application 115 may register and stitch together two or more intraoral scans generated thus far from the intraoral scan session to generate a growing 3D surface. In one embodiment, performing registration includes capturing 3D data of various points of a surface in multiple scans, and registering the scans by computing transformations between the scans. One or more 3D surfaces may be generated based on the registered and stitched together intraoral scans during the intraoral scanning. The one or more 3D surfaces may be output to a display so that a doctor or technician can view their scan progress thus far. As each new intraoral scan is captured and registered to previous intraoral scans and/or a 3D surface, the one or more 3D surfaces may be updated, and the updated 3D surface(s) may be output to the display. A view of the 3D surface(s) may be periodically or continuously updated according to one or more viewing modes of the intraoral scan application. In one viewing mode, the 3D surface may be continuously updated such that an orientation of the 3D surface that is displayed aligns with a field of view of the intraoral scanner (e.g., so that a portion of the 3D surface that is based on a most recently generated intraoral scan is approximately centered on the display or on a window of the display) and a user sees what the intraoral scanner sees. In one viewing mode, a position and orientation of the 3D surface is static, and an image of the intraoral scanner is optionally shown to move relative to the stationary 3D surface.

Intraoral scan application 115 may generate one or more 3D surfaces from intraoral scans, and may display the 3D surfaces to a user (e.g., a doctor) via a graphical user interface (GUI) during intraoral scanning. In embodiments, separate 3D surfaces are generated for the upper jaw and the lower jaw. This process may be performed in real time or near-real time to provide an updated view of the captured 3D surfaces during the intraoral scanning process. As scans are received, these scans may be registered and stitched to a 3D surface. Quality scores may be determined for various regions of the 3D surface based on one or more criteria as discussed in detail below. The quality scores may be continuously or periodically updated as information is added from further intraoral scans. As the quality scores gradually change, a visualization of the regions may change in accordance with the changes in the quality scores, enabling a user to have real time or near real time feedback on surface quality during scanning. Additionally, or alternatively, as scans are received the scanning process may be monitored to determine if a user is having trouble scanning any regions of a dental site (e.g., of the upper or lower dental arch). If a determination is made that a user is having trouble scanning a region of the dental site, then one or more remedial actions may be performed and/or one or more suggestions may be provided. Additionally, or alternatively, as scanning is being performed a zoom setting for displaying the 3D surface(s) may be dynamically determined based on one or more criteria, such as a velocity of the scanner and/or of a point of focus of the scanner. In embodiments, a user may select to enable or disable automatic zoom and/or automatic suggestions via the GUI. For example, the user may input a request for scanning assistance, which may cause automatic zoom and/or scanning suggestions to be enabled. These and other operations may be performed during scanning to improve a quality of the 3D surface(s), to speed up scanning, to help a user in trouble areas, and so on.

When a scan session or a portion of a scan session associated with a particular scanning role (e.g., upper jaw role, lower jaw role, bite role, etc.) is complete (e.g., all scans for an dental site or dental site have been captured), intraoral scan application 115 may generate a virtual 3D model of one or more scanned dental sites (e.g., of an upper jaw and a lower jaw). The final 3D model may be a set of 3D points and their connections with each other (i.e. a mesh). To generate the virtual 3D model, intraoral scan application 115 may register and stitch together the intraoral scans generated from the intraoral scan session that are associated with a particular scanning role. The registration performed at this stage may be more accurate than the registration performed during the capturing of the intraoral scans, and may take more time to complete than the registration performed during the capturing of the intraoral scans. In one embodiment, performing scan registration includes capturing 3D data of various points of a surface in multiple scans, and registering the scans by computing transformations between the scans. The 3D data may be projected into a 3D space of a 3D model to form a portion of the 3D model. The intraoral scans may be integrated into a common reference frame by applying appropriate transformations to points of each registered scan and projecting each scan into the 3D space.

In one embodiment, registration is performed for adjacent or overlapping intraoral scans (e.g., each successive frame of an intraoral video). Registration algorithms are carried out to register two adjacent or overlapping intraoral scans and/or to register an intraoral scan with a 3D model, which essentially involves determination of the transformations which align one scan with the other scan and/or with the 3D model. Registration may involve identifying multiple points in each scan (e.g., point clouds) of a scan pair (or of a scan and the 3D model), surface fitting to the points, and using local searches around points to match points of the two scans (or of the scan and the 3D model). For example, intraoral scan application 115 may match points of one scan with the closest points interpolated on the surface of another scan, and iteratively minimize the distance between matched points. Other registration techniques may also be used.

Intraoral scan application 115 may repeat registration for all intraoral scans of a sequence of intraoral scans to obtain transformations for each intraoral scan, to register each intraoral scan with previous intraoral scan(s) and/or with a common reference frame (e.g., with the 3D model). Intraoral scan application 115 may integrate intraoral scans into a single virtual 3D model by applying the appropriate determined transformations to each of the intraoral scans. Each transformation may include rotations about one to three axes and translations within one to three planes.

Intraoral scan application 115 may generate one or more 3D models from intraoral scans, and may display the 3D models to a user (e.g., a doctor) via a graphical user interface (GUI). The 3D models can then be checked visually by the doctor. The doctor can virtually manipulate the 3D models via the user interface with respect to up to six degrees of freedom (i.e., translated and/or rotated with respect to one or more of three mutually orthogonal axes) using suitable user controls (hardware and/or virtual) to enable viewing of the 3D model from any desired direction.

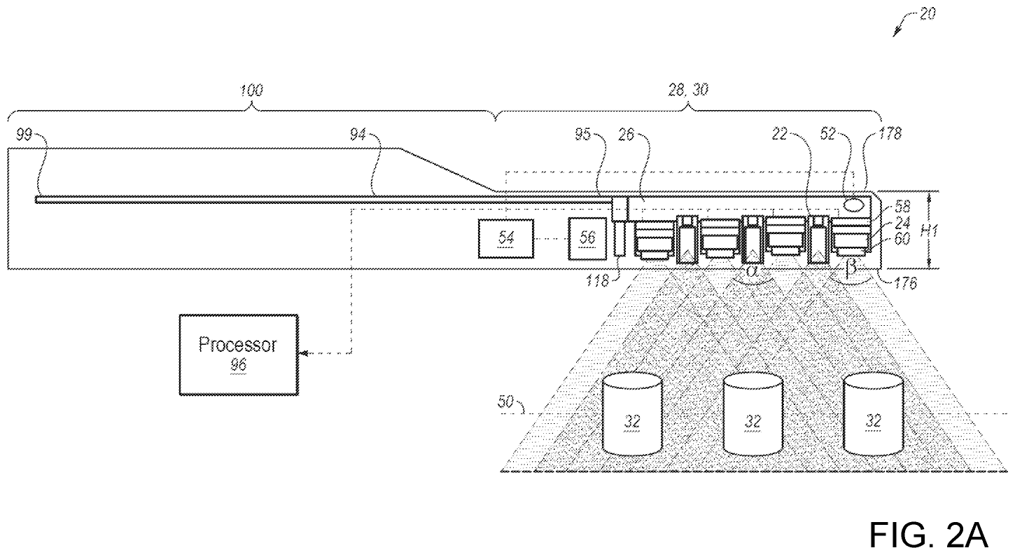

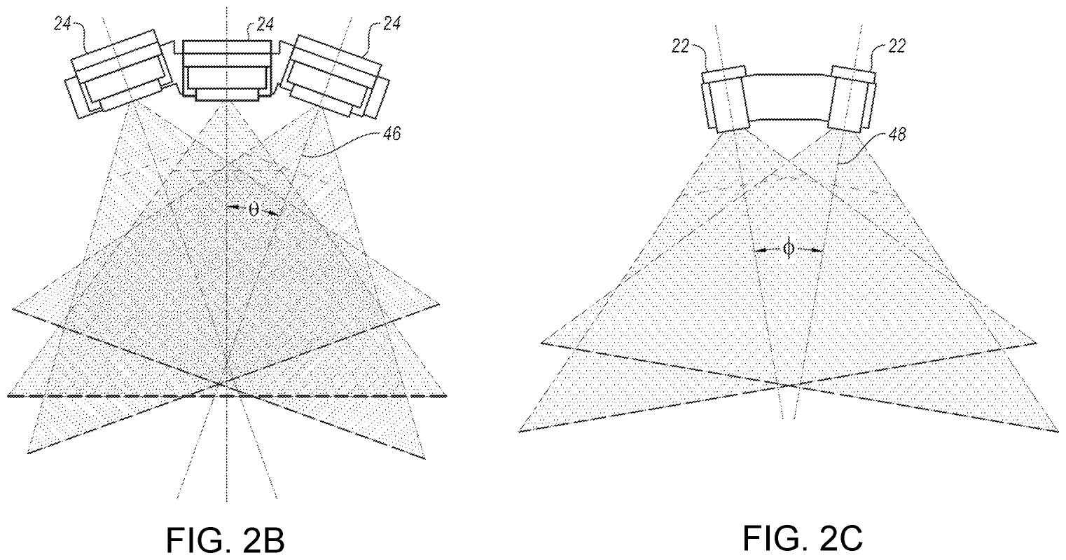

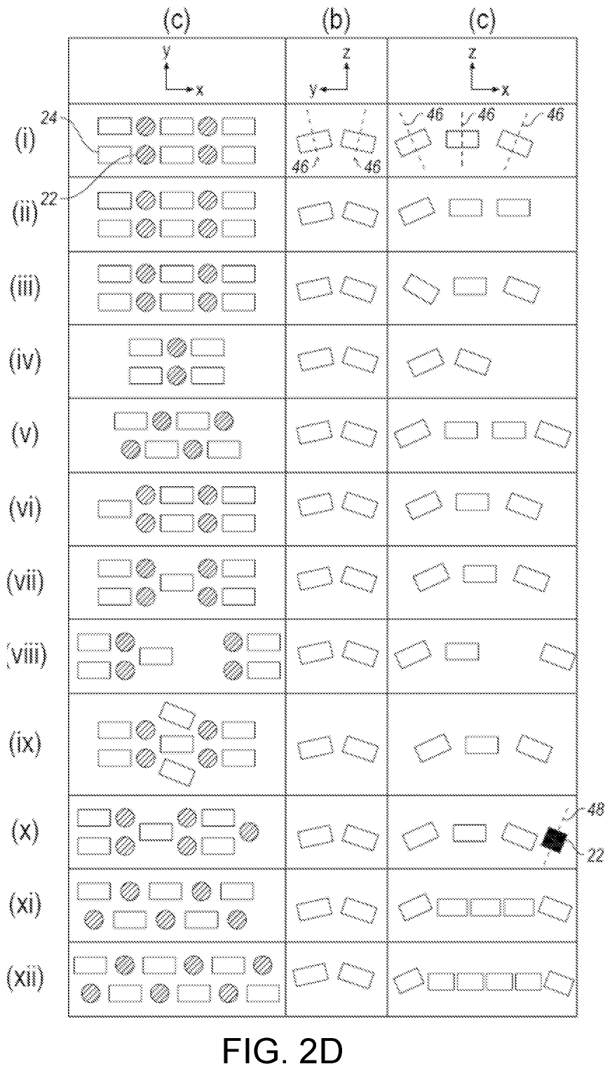

Reference is now made to FIG. 2A, which is a schematic illustration of an intraoral scanner 20 comprising an elongate handheld wand, in accordance with some applications of the present disclosure. The intraoral scanner 20 may correspond to intraoral scanner 150 of FIG. 1 in embodiments. Intraoral scanner 20 includes a plurality of structured light projectors 22 and a plurality of cameras 24 that are coupled to a rigid structure 26 disposed within a probe 28 at a distal end 30 of the intraoral scanner 20. In some applications, during an intraoral scanning procedure, probe 28 is inserted into the oral cavity of a subject or patient.

For some applications, structured light projectors 22 are positioned within probe 28 such that each structured light projector 22 faces an object 32 outside of intraoral scanner 20 that is placed in its field of illumination, as opposed to positioning the structured light projectors in a proximal end of the handheld wand and illuminating the object by reflection of light off a mirror and subsequently onto the object. Alternatively, the structured light projectors may be disposed at a proximal end of the handheld wand. Similarly, for some applications, cameras 24 are positioned within probe 28 such that each camera 24 faces an object 32 outside of intraoral scanner 20 that is placed in its field of view, as opposed to positioning the cameras in a proximal end of the intraoral scanner and viewing the object by reflection of light off a mirror and into the camera. This positioning of the projectors and the cameras within probe 28 enables the scanner to have an overall large field of view while maintaining a low profile probe. Alternatively, the cameras may be disposed in a proximal end of the handheld wand.

In some applications, cameras 24 each have a large field of view β (beta) of at least 45 degrees, e.g., at least 70 degrees, e.g., at least 80 degrees, e.g., 85 degrees. In some applications, the field of view may be less than 120 degrees, e.g., less than 100 degrees, e.g., less than 90 degrees. In one embodiment, a field of view β (beta) for each camera is between 80 and 90 degrees, which may be particularly useful because it provided a good balance among pixel size, field of view and camera overlap, optical quality, and cost. Cameras 24 may include an image sensor 58 and objective optics 60 including one or more lenses. To enable close focus imaging, cameras 24 may focus at an object focal plane 50 that is located between 1 mm and 30 mm, e.g., between 4 mm and 24 mm, e.g., between 5 mm and 11 mm, e.g., 9 mm-10 mm, from the lens that is farthest from the sensor. In some applications, cameras 24 may capture images at a frame rate of at least 30 frames per second, e.g., at a frame of at least 75 frames per second, e.g., at least 100 frames per second. In some applications, the frame rate may be less than 200 frames per second.

A large field of view achieved by combining the respective fields of view of all the cameras may improve accuracy due to reduced amount of image stitching errors, especially in edentulous regions, where the gum surface is smooth and there may be fewer clear high resolution 3D features. Having a larger field of view enables large smooth features, such as the overall curve of the tooth, to appear in each image frame, which improves the accuracy of stitching respective surfaces obtained from multiple such image frames.

Similarly, structured light projectors 22 may each have a large field of illumination a (alpha) of at least 45 degrees, e.g., at least 70 degrees. In some applications, field of illumination a (alpha) may be less than 120 degrees, e.g., than 100 degrees.

For some applications, in order to improve image capture, each camera 24 has a plurality of discrete preset focus positions, in each focus position the camera focusing at a respective object focal plane 50. Each of cameras 24 may include an autofocus actuator that selects a focus position from the discrete preset focus positions in order to improve a given image capture. Additionally or alternatively, each camera 24 includes an optical aperture phase mask that extends a depth of focus of the camera, such that images formed by each camera are maintained focused over all object distances located between 1 mm and 30 mm, e.g., between 4 mm and 24 mm, e.g., between 5 mm and 11 mm, e.g., 9 mm-10 mm, from the lens that is farthest from the sensor.

In some applications, structured light projectors 22 and cameras 24 are coupled to rigid structure 26 in a closely packed and/or alternating fashion, such that (a) a substantial part of each camera's field of view overlaps the field of view of neighboring cameras, and (b) a substantial part of each camera's field of view overlaps the field of illumination of neighboring projectors. Optionally, at least 20%, e.g., at least 50%, e.g., at least 75% of the projected pattern of light are in the field of view of at least one of the cameras at an object focal plane 50 that is located at least 4 mm from the lens that is farthest from the sensor. Due to different possible configurations of the projectors and cameras, some of the projected pattern may never be seen in the field of view of any of the cameras, and some of the projected pattern may be blocked from view by object 32 as the scanner is moved around during a scan.

Rigid structure 26 may be a non-flexible structure to which structured light projectors 22 and cameras 24 are coupled so as to provide structural stability to the optics within probe 28. Coupling all the projectors and all the cameras to a common rigid structure helps maintain geometric integrity of the optics of each structured light projector 22 and each camera 24 under varying ambient conditions, e.g., under mechanical stress as may be induced by the subject's mouth. Additionally, rigid structure 26 helps maintain stable structural integrity and positioning of structured light projectors 22 and cameras 24 with respect to each other.

Reference is now made to FIGS. 2B-2C, which include schematic illustrations of a positioning configuration for cameras 24 and structured light projectors 22 respectively, in accordance with some applications of the present disclosure. For some applications, in order to improve the overall field of view and field of illumination of the intraoral scanner 20, cameras 24 and structured light projectors 22 are positioned such that they do not all face the same direction. For some applications, such as is shown in FIG. 2B, a plurality of cameras 24 are coupled to rigid structure 26 such that an angle θ (theta) between two respective optical axes 46 of at least two cameras 24 is 90 degrees or less, e.g., 35 degrees or less. Similarly, for some applications, such as is shown in FIG. 2C, a plurality of structured light projectors 22 are coupled to rigid structure 26 such that an angle φ (phi) between two respective optical axes 48 of at least two structured light projectors 22 is 90 degrees or less, e.g., 35 degrees or less.

Reference is now made to FIG. 2D, which is a chart depicting a plurality of different configurations for the position of structured light projectors 22 and cameras 24 in probe 28, in accordance with some applications of the present disclosure. Structured light projectors 22 are represented in FIG. 2D by circles and cameras 24 are represented in FIG. 2D by rectangles. It is noted that rectangles are used to represent the cameras, since typically, each image sensor 58 and the field of view β (beta) of each camera 24 have aspect ratios of 1:2. Column (a) of FIG. 2D shows a bird's eye view of the various configurations of structured light projectors 22 and cameras 24. The x-axis as labeled in the first row of column (a) corresponds to a central longitudinal axis of probe 28. Column (b) shows a side view of cameras 24 from the various configurations as viewed from a line of sight that is coaxial with the central longitudinal axis of probe 28 and substantially parallel to a viewing axis of the intraoral scanner. Similarly to as shown in FIG. 2B, column (b) of FIG. 2D shows cameras 24 positioned so as to have optical axes 46 at an angle of 90 degrees or less, e.g., 35 degrees or less, with respect to each other. Column (c) shows a side view of cameras 24 of the various configurations as viewed from a line of sight that is perpendicular to the central longitudinal axis of probe 28.

Typically, the distal-most (toward the positive x-direction in FIG. 2D) and proximal-most (toward the negative x-direction in FIG. 2D) cameras 24 are positioned such that their optical axes 46 are slightly turned inwards, e.g., at an angle of 90 degrees or less, e.g., 35 degrees or less, with respect to the next closest camera 24. The camera(s) 24 that are more centrally positioned, i.e., not the distal-most camera 24 nor proximal-most camera 24, are positioned so as to face directly out of the probe, their optical axes 46 being substantially perpendicular to the central longitudinal axis of probe 28. It is noted that in row (xi) a projector 22 is positioned in the distal-most position of probe 28, and as such the optical axis 48 of that projector 22 points inwards, allowing a larger number of spots 33 projected from that particular projector 22 to be seen by more cameras 24.

In embodiments, the number of structured light projectors 22 in probe 28 may range from two, e.g., as shown in row (iv) of FIG. 2D, to six, e.g., as shown in row (xii). Typically, the number of cameras 24 in probe 28 may range from four, e.g., as shown in rows (iv) and (v), to seven, e.g., as shown in row (ix). It is noted that the various configurations shown in FIG. 2D are by way of example and not limitation, and that the scope of the present disclosure includes additional configurations not shown. For example, the scope of the present disclosure includes fewer or more than five projectors 22 positioned in probe 28 and fewer or more than seven cameras positioned in probe 28.

In an example application, an apparatus for intraoral scanning (e.g., an intraoral scanner 150) includes an elongate handheld wand comprising a probe at a distal end of the elongate handheld wand, at least two light projectors disposed within the probe, and at least four cameras disposed within the probe. Each light projector may include at least one light source configured to generate light when activated, and a pattern generating optical element that is configured to generate a pattern of light when the light is transmitted through the pattern generating optical element. Each of the at least four cameras may include a camera sensor (also referred to as an image sensor) and one or more lenses, wherein each of the at least four cameras is configured to capture a plurality of images that depict at least a portion of the projected pattern of light on an intraoral surface. A majority of the at least two light projectors and the at least four cameras may be arranged in at least two rows that are each approximately parallel to a longitudinal axis of the probe, the at least two rows comprising at least a first row and a second row.

In a further application, a distal-most camera along the longitudinal axis and a proximal-most camera along the longitudinal axis of the at least four cameras are positioned such that their optical axes are at an angle of 90 degrees or less with respect to each other from a line of sight that is perpendicular to the longitudinal axis. Cameras in the first row and cameras in the second row may be positioned such that optical axes of the cameras in the first row are at an angle of 90 degrees or less with respect to optical axes of the cameras in the second row from a line of sight that is coaxial with the longitudinal axis of the probe. A remainder of the at least four cameras other than the distal-most camera and the proximal-most camera have optical axes that are substantially parallel to the longitudinal axis of the probe. Each of the at least two rows may include an alternating sequence of light projectors and cameras.

In a further application, the at least four cameras comprise at least five cameras, the at least two light projectors comprise at least five light projectors, a proximal-most component in the first row is a light projector, and a proximal-most component in the second row is a camera.

In a further application, the distal-most camera along the longitudinal axis and the proximal-most camera along the longitudinal axis are positioned such that their optical axes are at an angle of 35 degrees or less with respect to each other from the line of sight that is perpendicular to the longitudinal axis. The cameras in the first row and the cameras in the second row may be positioned such that the optical axes of the cameras in the first row are at an angle of 35 degrees or less with respect to the optical axes of the cameras in the second row from the line of sight that is coaxial with the longitudinal axis of the probe.

In a further application, the at least four cameras may have a combined field of view of 25-45 mm along the longitudinal axis and a field of view of 20-40 mm along a z-axis corresponding to distance from the probe.

Returning to FIG. 2A, for some applications, there is at least one uniform light projector 118 (which may be an unstructured light projector that projects light across a range of wavelengths) coupled to rigid structure 26. Uniform light projector 118 may transmit white light onto object 32 being scanned. At least one camera, e.g., one of cameras 24, captures two-dimensional color images of object 32 using illumination from uniform light projector 118.

Processor 96 may run a surface reconstruction algorithm that may use detected patterns (e.g., dot patterns) projected onto object 32 to generate a 3D surface of the object 32. In some embodiments, the processor 96 may combine at least one 3D scan captured using illumination from structured light projectors 22 with a plurality of intraoral 2D images captured using illumination from uniform light projector 118 in order to generate a digital three-dimensional image of the intraoral three-dimensional surface. Using a combination of structured light and uniform illumination enhances the overall capture of the intraoral scanner and may help reduce the number of options that processor 96 needs to consider when running a correspondence algorithm used to detect depth values for object 32. In one embodiment, the intraoral scanner and correspondence algorithm described in U.S. application Ser. No. 16/446,181, filed Jun. 19, 2019, is used. U.S. application Ser. No. 16/446,181, filed Jun. 19, 2019, is incorporated by reference herein in its entirety. In embodiments, processor 96 may be a processor of computing device 105 of FIG. 1. Alternatively, processor 96 may be a processor integrated into the intraoral scanner 20.

For some applications, all data points taken at a specific time are used as a rigid point cloud, and multiple such point clouds are captured at a frame rate of over 10 captures per second. The plurality of point clouds are then stitched together using a registration algorithm, e.g., iterative closest point (ICP), to create a dense point cloud. A surface reconstruction algorithm may then be used to generate a representation of the surface of object 32.

For some applications, at least one temperature sensor 52 is coupled to rigid structure 26 and measures a temperature of rigid structure 26. Temperature control circuitry 54 disposed within intraoral scanner 20 (a) receives data from temperature sensor 52 indicative of the temperature of rigid structure 26 and (b) activates a temperature control unit 56 in response to the received data. Temperature control unit 56, e.g., a PID controller, keeps probe 28 at a desired temperature (e.g., between 35 and 43 degrees Celsius, between 37 and 41 degrees Celsius, etc.). Keeping probe 28 above 35 degrees Celsius, e.g., above 37 degrees Celsius, reduces fogging of the glass surface of intraoral scanner 20, through which structured light projectors 22 project and cameras 24 view, as probe 28 enters the intraoral cavity, which is typically around or above 37 degrees Celsius. Keeping probe 28 below 43 degrees, e.g., below 41 degrees Celsius, prevents discomfort or pain.

In some embodiments, heat may be drawn out of the probe 28 via a heat conducting element 94, e.g., a heat pipe, that is disposed within intraoral scanner 20, such that a distal end 95 of heat conducting element 94 is in contact with rigid structure 26 and a proximal end 99 is in contact with a proximal end 100 of intraoral scanner 20. Heat is thereby transferred from rigid structure 26 to proximal end 100 of intraoral scanner 20. Alternatively or additionally, a fan disposed in a handle region 174 of intraoral scanner 20 may be used to draw heat out of probe 28.

FIGS. 2A-2D illustrate one type of intraoral scanner that can be used for embodiments of the present disclosure. However, it should be understood that embodiments are not limited to the illustrated type of intraoral scanner. In one embodiment, intraoral scanner 150 corresponds to the intraoral scanner described in U.S. application Ser. No. 16/910,042, filed Jun. 23, 2020 and entitled “Intraoral 3D Scanner Employing Multiple Miniature Cameras and Multiple Miniature Pattern Projectors”, which is incorporated by reference herein. In one embodiment, intraoral scanner 150 corresponds to the intraoral scanner described in U.S. application Ser. No. 16/446,181, filed Jun. 19, 2019 and entitled “Intraoral 3D Scanner Employing Multiple Miniature Cameras and Multiple Miniature Pattern Projectors”, which is incorporated by reference herein.

In some embodiments an intraoral scanner that performs confocal focusing to determine depth information may be used. Such an intraoral scanner may include a light source and/or illumination module that emits light (e.g., a focused light beam or array of focused light beams). The light passes through a polarizer and through a unidirectional mirror or beam splitter (e.g., a polarizing beam splitter) that passes the light. The light may pass through a pattern before or after the beam splitter to cause the light to become patterned light. Along an optical path of the light after the unidirectional mirror or beam splitter are optics, which may include one or more lens groups. Any of the lens groups may include only a single lens or multiple lenses. One of the lens groups may include at least one moving lens.

The light may pass through an endoscopic probing member, which may include a rigid, light-transmitting medium, which may be a hollow object defining within it a light transmission path or an object made of a light transmitting material, e.g. a glass body or tube. In one embodiment, the endoscopic probing member includes a prism such as a folding prism. At its end, the endoscopic probing member may include a mirror of the kind ensuring a total internal reflection. Thus, the mirror may direct the array of light beams towards a teeth segment or other object. The endoscope probing member thus emits light, which optionally passes through one or more windows and then impinges on to surfaces of intraoral objects.

The light may include an array of light beams arranged in an X-Y plane, in a Cartesian frame, propagating along a Z axis, which corresponds to an imaging axis or viewing axis of the intraoral scanner. As the surface on which the incident light beams hits is an uneven surface, illuminated spots may be displaced from one another along the Z axis, at different (Xᵢ, Yᵢ) locations. Thus, while a spot at one location may be in focus of the confocal focusing optics, spots at other locations may be out-of-focus. Therefore, the light intensity of returned light beams of the focused spots will be at its peak, while the light intensity at other spots will be off peak. Thus, for each illuminated spot, multiple measurements of light intensity are made at different positions along the Z-axis. For each of such (Xᵢ, Yᵢ) location, the derivative of the intensity over distance (Z) may be made, with the Zᵢ yielding maximum derivative, Z₀, being the in-focus distance.

The light reflects off of intraoral objects and passes back through windows (if they are present), reflects off of the mirror, passes through the optical system, and is reflected by the beam splitter onto a detector. The detector is an image sensor having a matrix of sensing elements each representing a pixel of the scan or image. In one embodiment, the detector is a charge coupled device (CCD) sensor. In one embodiment, the detector is a complementary metal-oxide semiconductor (CMOS) type image sensor. Other types of image sensors may also be used for detector. In one embodiment, the detector detects light intensity at each pixel, which may be used to compute height or depth.

Alternatively, in some embodiments an intraoral scanner that uses stereo imaging is used to determine depth information.

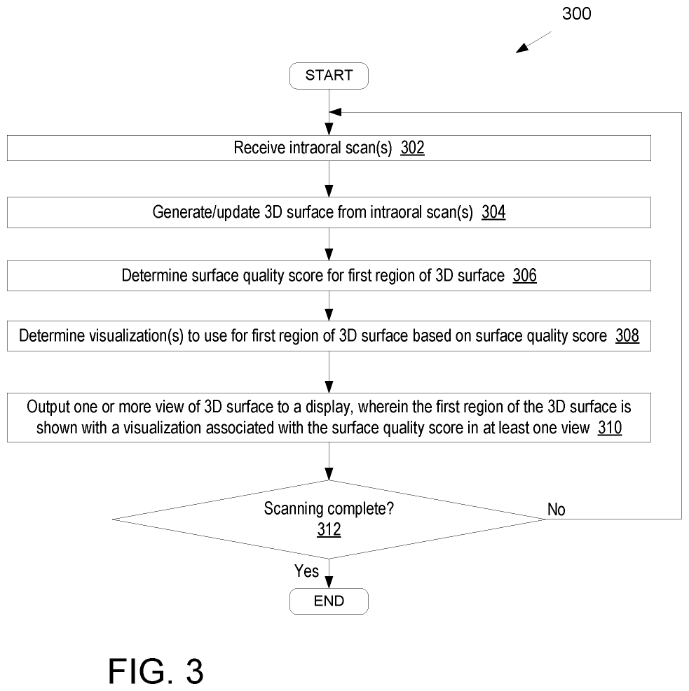

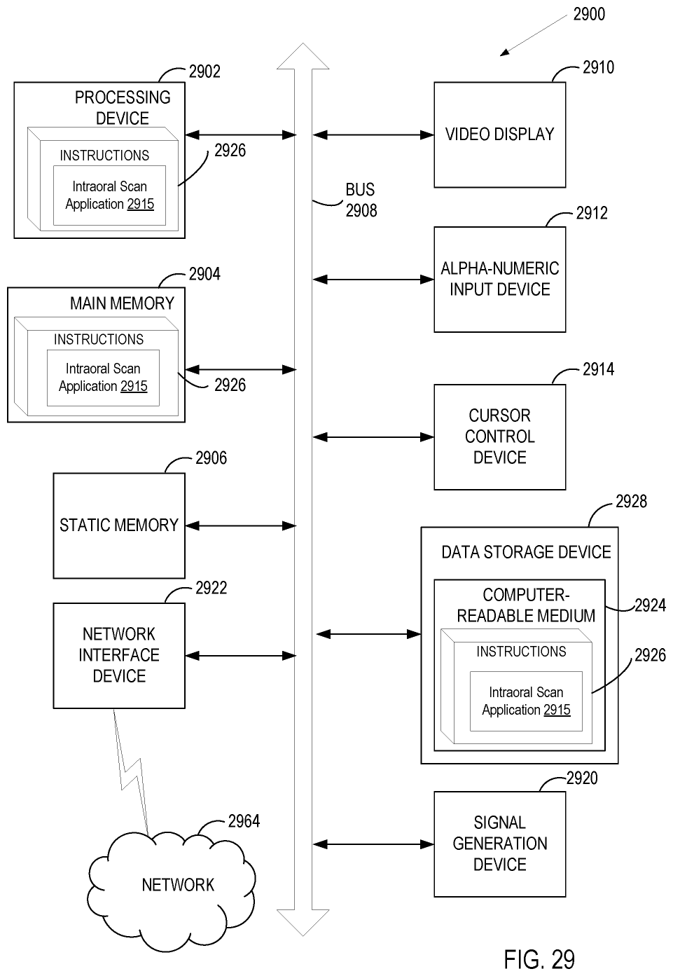

FIGS. 3-8, 10-11, 14A-16B, 19-20, and 25-27 are flow charts illustrating various methods related to providing user feedback during intraoral scanning to assist in the intraoral scanning. The methods may be performed by a processing logic that may comprise hardware (e.g., circuitry, dedicated logic, programmable logic, microcode, etc.), software (e.g., instructions run on a processing device to perform hardware simulation), firmware, or a combination thereof. In one embodiment, at least some operations of the methods are performed by a computing device of a scanning system and/or by a server computing device (e.g., by computing device 105 of FIG. 1 or computing device 2900 of FIG. 29).

FIG. 3 is a flow chart for a method 300 of displaying a 3D surface of a dental site during intraoral scanning in a manner that shows surface quality, in accordance with embodiments of the present disclosure. At block 302 of method 300, processing logic receives one or more intraoral scans of a dental site. Processing logic may additionally receive one or more two-dimensional (2D) images of the dental site, which may include color 2D images, near infrared (NIR) 2D images, 2D images generated under ultraviolet light, and so on. Each of the intraoral scans may include three-dimensional information about a captured portion of the dental site. For example, each intraoral scan may include point clouds. In embodiments, each intraoral scan includes three dimensional information (e.g., x,y,z coordinates) for multiple points on a dental surface. Each of the multiple points may correspond to a spot or feature of structured light that was projected by a structured light projector of the intraoral scanner onto the dental site and that was captured in images generated by one or more cameras of the intraoral scanner.

At block 304, processing logic generates a 3D surface representing the scanned dental site using the one or more received intraoral scans. This may include registering and stitching together multiple intraoral scans and/or registering and stitching one or more intraoral scans to an already generated 3D surface to update the 3D surface. The registration and stitching process may be performed as described in greater detail above. As further intraoral scans are received, those intraoral scans may be registered and stitched to the 3D surface to add information for more regions/portions of the 3D surface and/or to improve the quality of one or more regions/portions of the 3D surface that are already present. In some embodiments, the generated surface is an approximated surface that may be of lower quality than a surface that will be later calculated.

At block 306, processing logic determines surface quality scores for one or more regions of the 3D surface. In some embodiments, the 3D surface is divided into a plurality of regions, which may have a same size or different sizes. For example, in one embodiment the 3D surface is divided into approximately mm sized regions (e.g., 1 mm square region, 1 mm diameter region, a 1 mm cube region, etc.). Alternatively, larger or smaller regions may be used. Surface quality scores may be determined for each region. This may include determining a first surface quality score for a first region of the 3D surface. In embodiments, processing logic determines surface quality scores for individual points on the surface and/or for groups of points on the surface. Accordingly, the size of a region may vary from a single point location to an entire tooth, a group of multiple teeth, and so on. In some embodiments, a region is determined by grouping together adjacent and/or proximate points that have the same or similar surface quality scores.

Multiple techniques may be used to determine surface quality scores for regions of the 3D surface. In some instances, intraoral scans are scored, and the scores from the intraoral scans are used to determine scores for associated regions that were generated based on those intraoral scans. In some instances, individual points of intraoral scans are scored, and the determined scores for those points are applied to associated points on the 3D surface. In some instances, 2D images and/or points on 2D images are scored, and those scores are applied to associated points on the 3D surface. In some instances, regions are scored at least in part on a number of color and/or NIRI images depicting the regions. A number of color and/or NIRI images that depict a region may be determined, and if the number is below a threshold a user may be notified to generate more images and/or a low surface quality score may be indicated. In some instances, individual points on the 3D surface are scored and regions are determined based on groupings of similarly scored points. In some embodiments, data associated with a region of a 3D surface of a dental site is input into a trained machine learning model, which outputs one or more surface quality scores for the region. In some instances, a number of measured data points in a region is counted, and the surface quality score for the region is determined based at least on the number of points (e.g., number of points per mm²).

Different scoring criteria may be applied to determine surface quality scores for regions of a 3D surface. Some information that may be used in computing a surface quality score includes point density for a region, quality scores for the points that make up a region, roughness of the 3D surface at a region, distance between the region and one or more cameras that generated an intraoral scan comprising points in the region, distance between multiple cameras that captured one or more points in the region, distance between a structured light projector and a camera that captured on or more points in the region, a number of cameras that captured one or more points in the region, spot sizes of one or more points in the region, an angle of the 3D surface at the region relative to the intraoral scanner, a type of material of the dental site at the region, and so on. Multiple different techniques for determining surface quality scores are discussed with reference to the following figures, any of which may be applied at block 306.

At block 308, processing logic determines one or more visualizations to use for the various regions of the 3D surface based on surface quality scores assigned to those regions. This may include determining a first visualization to user for the first region of the 3D surface based on the surface quality score determined for the first region. In some embodiments, processing logic inputs the surface quality score into a function, and the function outputs a visualization to use for the surface quality score. In some embodiments, processing logic performs a lookup in a lookup table that associates different surface quality scores to different visualizations. For example, the lookup table may include multiple entries, each of which may associate a particular visualization with a range of surface quality score values. In some embodiments, different rubrics are used for determining visualizations to select for surface quality scores, where the rubrics may be selected based on properties of a region of the 3D surface and/or based on a classification of the region of the 3D surface. For example, a region that is classified as a preparation tooth may be associated with a first rubric and a region that is classified as a standard tooth may be associated with a second rubric. In another example, a region that is classified as a margin line may be associated with a first rubric, a region that is classified as a preparation tooth may be associated with a second rubric, and a region that is classified as a standard tooth may be associated with a third rubric.

In one example, surface quality scores are associated with color values. Accordingly, a color may be selected for a region based on the surface quality score of that region. For example, low surface quality scores may be shown with shades of red, slightly higher surface quality scores may be shown with shades of orange, medium surface quality scores may be shown with shades of yellow, high surface quality scores may be shown with shades of green, and so on. In one example, surface quality scores are associated with transparency values. For example, low surface quality scores may be associated with high transparency values and high surface quality scores may be associated with low transparency values. Accordingly, as the surface quality score improves for a region, the level of transparency used to show that region may gradually decrease. In one example, surface quality scores may be associated with a blinking or flashing frequency. For example, low surface quality scores may be associated with a high flashing frequency, and high surface quality scores may be associated with a low flashing frequency such as with no flashing. Any of these visualization techniques and/or other visualization techniques may be used to represent surface quality of regions of the 3D surface.

At block 310, processing logic outputs one or more views of the 3D surface to a display. Each region of the 3D surface may be displayed with a respective visualization that was determined for that region based on a surface quality score of the region. Accordingly, the first region may be shown with a visualization associated with the surface quality score for the first region in the one or more view.

At block 312, processing logic determines whether scanning is complete. If scanning is not complete (e.g., more intraoral scans are still being generated), the method returns to block 302 and additional intraoral scans are received and processed. This may result in updates to the 3D surface at block 304 and possibly to changes in the surface quality scores for one or more regions of the 3D surface at block 306. In embodiments, the surface quality scores for regions may change gradually over time as further intraoral scans with information for those regions are received and processed, and the 3D surface is updated based on those intraoral scans. As the surface quality scores are gradually changed the visualizations that correlate to those surface quality scores are likewise gradually changed. This enables a user of the intraoral scanner to receive real-time feedback on surface quality, and to know whether they are making progress during the scanning or if they are stuck at a region without improving the surface quality of that region.

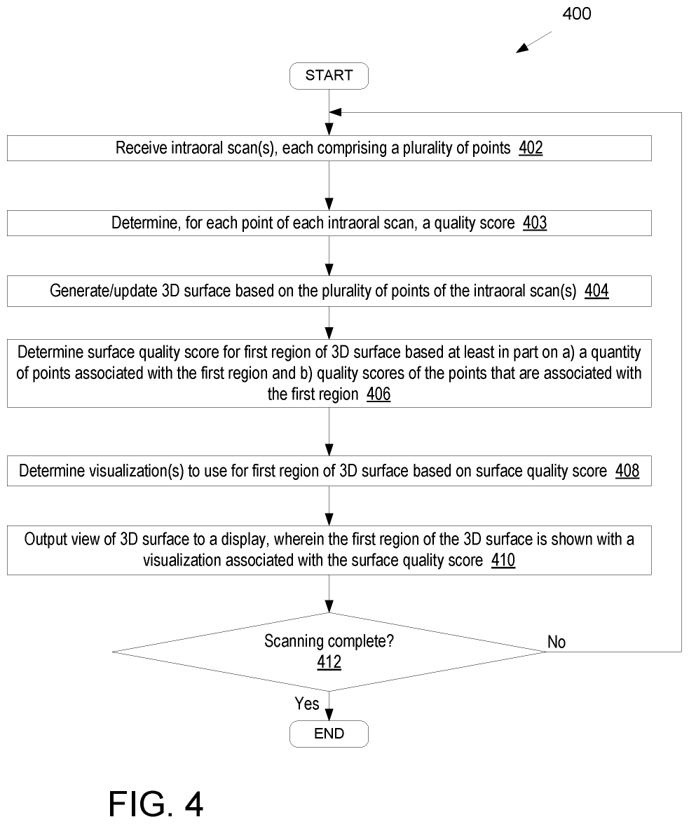

FIG. 4 is a flow chart for a method 400 of displaying a 3D surface of a dental site during intraoral scanning in a manner that shows surface quality, in accordance with embodiments of the present disclosure. At block 402 of method 400, processing logic receives one or more intraoral scans of a dental site. Processing logic may additionally receive one or more two-dimensional (2D) images of the dental site, which may include color 2D images, near infrared (NIR) 2D images, 2D images generated under ultraviolet light, and so on. Each of the intraoral scans may include three-dimensional information about a captured portion of the dental site. For example, each intraoral scan may include point clouds. In embodiments, each intraoral scan includes three dimensional information (e.g., x,y,z coordinates) for multiple points on a dental surface. Each of the multiple points may correspond to a spot or feature of structured light that was projected by a structured light projector of the intraoral scanner onto the dental site and that was captured in images generated by one or more cameras of the intraoral scanner.

At block 403, processing logic determines quality scores for each point in received intraoral scans. Multiple techniques may be used to determine quality scores for points in intraoral scans. In some embodiments, an intraoral scan is input into a trained machine learning model, which outputs one or more quality scores for each of the points in the intraoral scan. Different scoring criteria may be applied to determine quality scores for points in an intraoral scan. Some information that may be used in computing a quality score for a point include distance between the point and one or more cameras that generated the intraoral scan, distance between multiple cameras that captured the point, distance between a structured light projector that projected the point and a camera that captured the point, a number of cameras that captured the, a spot size associated with the point, an angle of the 3D surface at the point relative to the intraoral scanner, a type of material of the dental site at the point, and so on. Multiple different techniques for determining quality scores are discussed with reference to the following figures, any of which may be applied at block 403.

At block 404, processing logic may generate a 3D surface representing the scanned dental site using the one or more received intraoral scans. This may include registering and stitching together multiple intraoral scans and/or registering and stitching one or more intraoral scans to an already generated 3D surface to update the 3D surface. The registration and stitching process may be performed as described in greater detail above. With the stitching, information for one or more points from the intraoral scans may be added to the 3D surface. As further intraoral scans are received, those intraoral scans may be registered and stitched to the 3D surface to add information for more points to the 3D surface.

At block 406, processing logic determines surface quality scores for one or more regions of the 3D surface. The surface quality score for a region may be determined at least in part on a) a quantity of points associated with the region and b) quality scores of the points that are associated with the region. Other criteria may also be used for the scoring, such as a surface roughness at the region. In embodiments, the size of a region may vary from a single point location to an entire tooth, a group of multiple teeth, and so on. In some embodiments, a region is determined by grouping together adjacent and/or proximate points that have the same or similar surface quality scores. Multiple different techniques for determining surface quality scores are discussed with reference to the following figures, any of which may be applied at block 406.

In some embodiments, processing logic determines surface quality scores for regions without actually generating the 3D surface that includes those regions. Processing logic may generate such surface quality scores based on the quality scores of points from multiple intraoral scans and/or 2D images that depict the same general region or location of a dental site. By estimating surface quality scores without actually generating the 3D surface a speed of estimating the surface quality scores may be increased.

In some embodiments, intraoral scans initially lack depth (z) information, and the depth information is determined using a correspondence algorithm. Computation of the depth information using the correspondence algorithm may be a processor intensive task. Accordingly, in some embodiments the quality scores for points is determined before depth information is determined for those points (e.g., based on 2D information). This may provide rough quality values for points. These rough quality values may then be used to determine a surface quality score for a region very quickly. Then after the depth information for the points is determined updated quality scores for the points may be computed that has a greater accuracy, and the updated quality scores may be used to refine the surface quality score for a region of the 3D surface.

At block 408, processing logic determines one or more visualizations to use for the various regions of the 3D surface based on surface quality scores assigned to those regions. This may include determining a first visualization to use for the first region of the 3D surface based on the surface quality score determined for the first region. In some embodiments, processing logic inputs the surface quality score into a function, and the function outputs a visualization to use for the surface quality score. In some embodiments, processing logic performs a lookup in a lookup table that associates different surface quality scores to different visualizations. For example, the lookup table may include multiple entries, each of which may associate a particular visualization with a range of surface quality score values. In some embodiments, different rubrics are used for determining visualizations to select for surface quality scores, where the rubrics may be selected based on properties of a region of the 3D surface and/or based on a classification of the region of the 3D surface. For example, a region that is classified as a preparation tooth may be associated with a first rubric and a region that is classified as a standard tooth may be associated with a second rubric. In another example, a region that is classified as a margin line may be associated with a first rubric, a region that is classified as a preparation tooth may be associated with a second rubric, and a region that is classified as a standard tooth may be associated with a third rubric.

At block 410, processing logic outputs one or more views of the 3D surface to a display. Each region of the 3D surface may be displayed with a respective visualization that was determined for that region based on a surface quality score of the region. Accordingly, the first region may be shown with a visualization associated with the surface quality score for the first region in the one or more view.

At block 412, processing logic determines whether scanning is complete. If scanning is not complete (e.g., more intraoral scans are still being generated), the method returns to block 402 and additional intraoral scans are received and processed. This may result in updates to the 3D surface at block 404 and possibly to changes in the surface quality scores for one or more regions of the 3D surface at block 406. In embodiments, the surface quality scores for regions may change gradually over time as further intraoral scans with information for those regions are received and processed, and the 3D surface is updated based on those intraoral scans. As the surface quality scores are gradually changed the visualizations that correlate to those surface quality scores are likewise gradually changed. This enables a user of the intraoral scanner to receive real-time feedback on surface quality, and to know whether they are making progress during the scanning or if they are stuck at a region without improving the surface quality of that region.

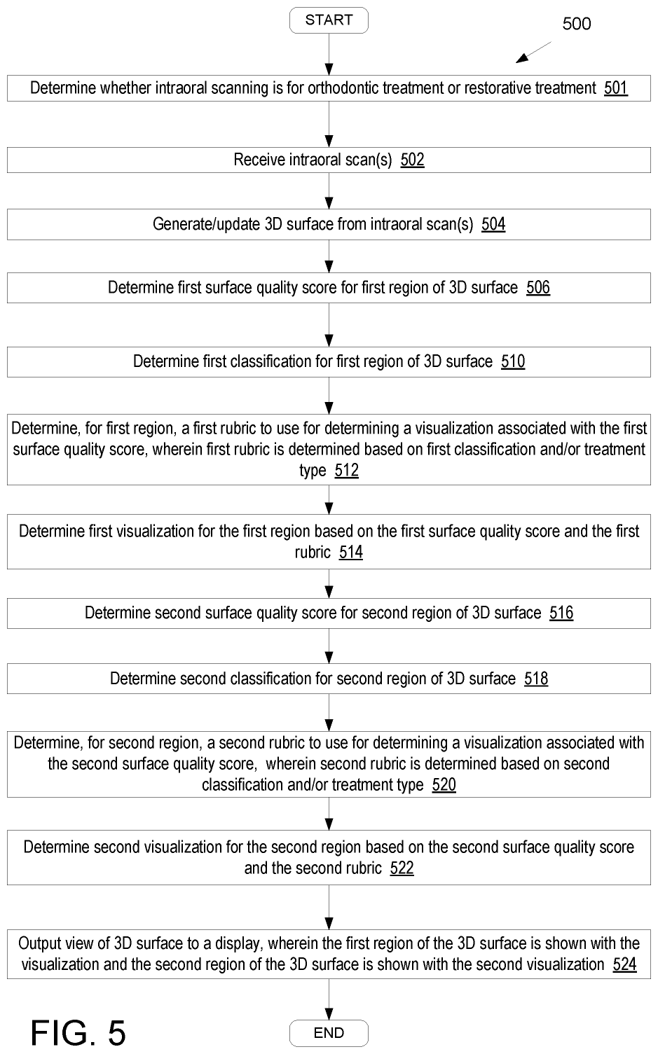

Different rubrics may be applied to translate surface quality scores into different visualizations in embodiments. Each of the rubrics may be associated with a particular type of treatment to be performed, on a class of dental surface being scanned, and/or on information. FIG. 5 is a flow chart for a method 500 of displaying a 3D surface of a dental site during intraoral scanning in a manner that shows different surface quality scores of different regions using different rubrics, in accordance with embodiments of the present disclosure. At block 501 of method 500, processing logic may determine whether intraoral scanning is for orthodontic treatment or for restorative treatment. In embodiments, different rubrics for translating surface quality scores into visualizations may be applied for different types of treatment. For example, different surface quality thresholds may be used to assess the sufficiency of surface quality for 3D surfaces generated for orthodontic treatment than for 3D surfaces generated for restorative treatment.

At block 502, processing logic receives one or more intraoral scans of a dental site. Processing logic may additionally receive one or more two-dimensional (2D) images of the dental site, which may include color 2D images, near infrared (NIR) 2D images, 2D images generated under ultraviolet light, and so on. Each of the intraoral scans may include three-dimensional information about a captured portion of the dental site. For example, each intraoral scan may include point clouds. In embodiments, each intraoral scan includes three dimensional information (e.g., x,y,z coordinates) for multiple points on a dental surface. Each of the multiple points may correspond to a spot or feature of structured light that was projected by a structured light projector of the intraoral scanner onto the dental site and that was captured in images generated by one or more cameras of the intraoral scanner.

At block 504, processing logic generates a 3D surface representing the scanned dental site using the one or more received intraoral scans. This may include registering and stitching together multiple intraoral scans and/or registering and stitching one or more intraoral scans to an already generated 3D surface to update the 3D surface. The registration and stitching process may be performed as described in greater detail above. As further intraoral scans are received, those intraoral scans may be registered and stitched to the 3D surface to add information for more regions/portions of the 3D surface and/or to improve the quality of one or more regions/portions of the 3D surface that are already present.

At block 506, processing logic determines surface quality scores for one or more regions of the 3D surface. This may include determining a first surface quality score for a first region of the 3D surface. In embodiments, processing logic determines surface quality scores for individual points on the surface and/or for groups of points on the surface. Accordingly, the size of a region may vary from a single point location to an entire tooth, a group of multiple teeth, and so on. In some embodiments, a region is determined by grouping together adjacent and/or proximate points that have the same or similar surface quality scores, as is discussed in greater detail with reference to other figures.

At block 510, processing logic may determine classifications for one or more regions of the 3D surface, which may include determining a first classification for a first region of the 3D surface. In one embodiment, a user manually marks a region of the 3D surface that is a preparation tooth. Alternatively, a user may indicate that the user is about to start scanning a preparation tooth, and a region scanned after receiving such user input may be marked as a preparation tooth. In one embodiment, the 3D surface is classified and/or segmented using one or more trained machine learning models. In some embodiments, processing logic uses one or more trained machine learning models (e.g., a neural network) trained to perform classification of dental sites, where at least one class is for a restorative object. Other classes of dental objects that may be identified include teeth, gums, preparation teeth (which may be considered a type of restorative object), margin line, upper palate, tongue, lips, gum-tooth line (referred to as emergent profile), and so on. In embodiments, the machine learning model receives data associated with a region of a 3D surface, which may include the 3D surface, one or more intraoral scans depicting the region, one or more 2D images depicting the region, one or more height maps of the region, etc. The machine learning model may process the data and output a dental object classification for the region. The trained machine learning model(s) may perform image level classification/scan level classification, may perform pixel-level classification, or may perform classification of groups of pixels. In embodiments, classification is performed using a trained machine learning model such as is discussed in U.S. application Ser. No. 17/230,825, filed Apr. 14, 2021, which is incorporated by reference herein in its entirety.

At block 512, processing logic determines a first rubric to use for determining a visualization associated with the first surface quality score of the first region. The first rubric may be determined based on at least one of the type of treatment to be performed (e.g., restorative vs. orthodontic treatment) or the dental object class determined for the first region. For example, the accuracy of certain regions of a dental arch may be of particular importance when performing intraoral scanning. In particular, for restorative treatment one or more teeth may be ground down for form a preparation tooth having a margin line. In general the level of accuracy required for the preparation tooth is greater than the level of accuracy required for surrounding teeth for production of a dental prosthetic (e.g., a crown, bridge, cap, etc.) that properly fits onto the preparation tooth. Additionally, the level of accuracy required for the margin line of the preparation tooth may be greater than the level of accuracy required for a remainder of the preparation tooth. Accordingly, different rubrics may be applied depending on the type of treatment and/or the dental object class. Each rubric may include one or more surface quality thresholds (e.g., an upper surface quality threshold where any region having a surface quality that at least meets the upper surface quality threshold is considered to be sufficient), one or more functions for determining visualizations based on surface quality scores, one or more lookup tables for determining visualizations based on surface quality scores, and so on. Accordingly, the same surface quality score may translate to different visualizations depending on the rubric applied to that surface quality score. In example, a surface quality score may be sufficient for a standard tooth and may be shown in green when applied to the standard tooth, and that same surface quality score may be insufficient for a preparation tooth and may be shown in yellow, orange or red when applied to a preparation tooth.

At block 514, processing logic determines a first visualization for the first region based on the first surface quality score and the first rubric. This may include determining one or more functions, look-up tables, thresholds, etc. from the first rubric to apply to the first surface quality score. The first rubric may indicate one or more first techniques for visualizing surface quality scores. For example, the first rubric may indicate that surface quality scores are to be shown based on a first one of color, texture, shading, transparency, flashing, pointers, arrows, flagging, etc. In some embodiments, the first dental object class is a tooth, and a second dental object is gingiva. How to visualize the region of the 3D surface based on the surface quality score may differ for different types of tissue (e.g., tooth tissue vs. gingival tissue). Since gingival tissue is generally of lesser importance than tooth tissue for orthodontic treatment, a low surface quality score for gingiva may still be shown with a visualization that indicates a high or sufficient surface quality score. Similarly, a palatal region may be of lesser importance than tooth tissue, and low surface quality scores may be shown as sufficient. In an embodiment, different rubrics are associated with different quality thresholds. For example, a surface quality threshold of 40 points per mm³ may be used for teeth, a surface quality threshold of 20 points per mm³ may be used for gingiva, and a surface quality threshold of 10 points per mm³ may be used for a palate. A tooth region with 30 points per mm³ may be shown in yellow or orange for example (representing a medium quality), whereas a gingival or palate region with 30 points per mm³ may be shown in green (representing a high quality). For some treatments, such as orthodontic treatment, a gum-tooth line (line where the tooth intersects the gingiva) may be important for proper fit of aligners. Accordingly, in some instances a dental object class of tooth-gum line may be assigned to a region, and a rubric associated with the tooth-gum line may have high thresholds for surface quality values. For example, a tooth-gum line may have a surface quality threshold of 50 points per mm³.

At block 516, processing logic determines a second surface quality score for a second region of the 3D surface. In one embodiment, the operations of block 516 are performed at block 506. At block 518, processing logic may determine a second classification for the second region, where the second classification for the second region is different from the first classification for the first region. In one embodiment, the operations of block 518 are performed at block 510 using the one or more trained machine learning models.

At block 520, processing logic determines a second rubric to use for determining a visualization associated with the second surface quality score of the second region. The second rubric may be determined based on at least one of the type of treatment to be performed (e.g., restorative vs. orthodontic treatment) or the dental object class determined for the second region. In embodiments, the second rubric to be used for the second region is different from the first rubric to be used for the first region.

At block 522, processing logic determines a second visualization for the second region based on the second surface quality score and the second rubric. This may include determining one or more functions, look-up tables, thresholds, etc. from the second rubric to apply to the second surface quality score. The second rubric may indicate one or more second techniques for visualizing surface quality scores. For example, the second rubric may indicate that surface quality scores are to be shown based on a second one of color, texture, shading, transparency, flashing, pointers, arrows, flagging, etc. For example, surface quality scores in the first region may be depicted using color and/or transparency level, and surface quality scores in the second region may be depicted using flashing.

At block 524, processing logic outputs one or more views of the 3D surface to a display. Each region of the 3D surface may be displayed with a respective visualization that was determined for that region based on a surface quality score of the region and/or a rubric determined to apply to that region. Accordingly, the first region may be shown with the first visualization associated with the first surface quality score as applied to one or more criteria of the first rubric and the second region may be shown with the second visualization associated with the second surface quality score as applied to one or more criteria of second first rubric. In an example, the first surface quality score of the first region may be the same as the second surface quality score of the second region. However, due to application of different rubrics to the first region and the second region, the first visualization used for the first region may be different from the second visualization used for the second region in spite of the first and second regions having the same surface quality scores.

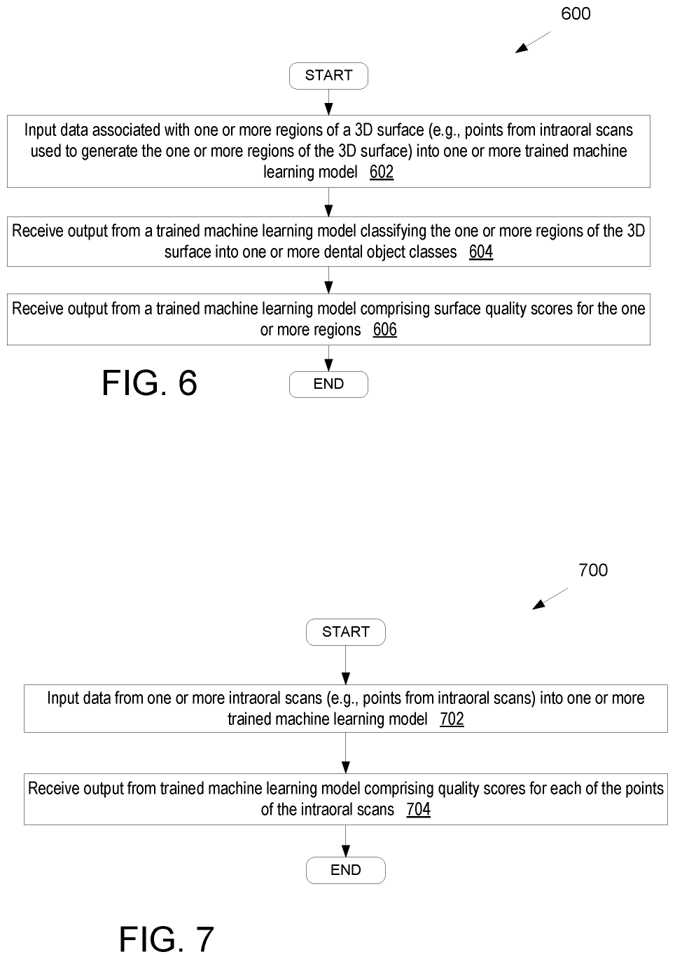

FIG. 6 is a flow chart for a method 600 of classifying regions of a 3D surface of a dental site and determining surface quality scores associated with the regions using one or more trained machine learning model, in accordance with embodiments of the present disclosure. At block 602 of method 600, processing logic inputs data associated with one or more regions of a 3D surface into one or more trained machine learning models.

In one embodiment, at block 604 processing logic receives an output from the one or more machine learning models classifying one or more regions of the 3D surface (and/or of an intraoral scan and/or dental site) into one or more dental classes. In one embodiment, the one or more trained machine learning models are trained to perform classification and/or segmentation of images, intraoral scans and/or 3D surfaces into dental classes. In embodiments, processing logic may input intraoral scans, 2D images, the 3D surface, projections of the 3D surface onto one or more planes, points from the 3D surface, and/or other data into the trained machine learning model(s). One implementation uses a deep neural network to learn how to map an input image, intraoral scan and/or 3D surface to human labeled dental classes, where the dental classes include regular teeth and one or more restorative objects. The result of this training is a trained machine learning model that can predict labels directly from input scan data and/or 3D surface data. Input data may be individual intraoral scans (e.g., height maps), 3D surface data (e.g., a 3D surface from multiple scans or a projection of such a 3D surface onto a plane) and/or or other images (e.g., color images and/or NIRI images). Such data may be available in real time while scanning. Additionally, intraoral scan data associated with an individual scan may be large enough (e.g., scanner may have a large enough FOV) as to include at least one tooth and its surroundings. Given an input based on a single intraoral scan, the trained neural network can predict if the scan (e.g., height map) contains any of the dental classes described above. The nature of such prediction may be probabilistic: for every class there is a probability of it being presented on the intraoral scan. Such approach allows the system to identify areas on a 3D surface and/or a 3D model generated from the intraoral scan that relate to restorative objects and thus should be treated differently than natural teeth.

In one embodiment, at block 606 processing logic receives an output from one or more trained machine learning models comprising surface quality scores for one or more regions of the 3D surface and/or for one or more intraoral scans.

In embodiments, one or more machine learning models are trained to perform one or both of the operations of block 604 and 606. Each task may be performed by a separate machine learning model. Alternatively, a single machine learning model may perform each of the tasks or a subset of the tasks. In an example, one or a few machine learning models may be trained, where the trained ML model is a single shared neural network that has multiple shared layers and multiple higher level distinct output layers, where each of the output layers outputs a different prediction, classification, identification, etc.

One type of machine learning model that may be used to perform some or all of the above tasks is an artificial neural network, such as a deep neural network. Artificial neural networks generally include a feature representation component with a classifier or regression layers that map features to a desired output space. A convolutional neural network (CNN), for example, hosts multiple layers of convolutional filters. Pooling is performed, and non-linearities may be addressed, at lower layers, on top of which a multi-layer perceptron is commonly appended, mapping top layer features extracted by the convolutional layers to decisions (e.g. classification outputs). Deep learning is a class of machine learning algorithms that use a cascade of multiple layers of nonlinear processing units for feature extraction and transformation. Each successive layer uses the output from the previous layer as input. Deep neural networks may learn in a supervised (e.g., classification) and/or unsupervised (e.g., pattern analysis) manner. Deep neural networks include a hierarchy of layers, where the different layers learn different levels of representations that correspond to different levels of abstraction. In deep learning, each level learns to transform its input data into a slightly more abstract and composite representation. In an image recognition application, for example, the raw input may be a matrix of pixels; the first representational layer may abstract the pixels and encode edges; the second layer may compose and encode arrangements of edges; the third layer may encode higher level shapes (e.g., teeth, lips, gums, etc.); and the fourth layer may recognize a scanning role. Notably, a deep learning process can learn which features to optimally place in which level on its own. The “deep” in “deep learning” refers to the number of layers through which the data is transformed. More precisely, deep learning systems have a substantial credit assignment path (CAP) depth. The CAP is the chain of transformations from input to output. CAPs describe potentially causal connections between input and output. For a feedforward neural network, the depth of the CAPs may be that of the network and may be the number of hidden layers plus one. For recurrent neural networks, in which a signal may propagate through a layer more than once, the CAP depth is potentially unlimited.

Training of a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many labeled inputs in the training dataset yields a network that can produce correct output when presented with inputs that are different than the ones present in the training dataset. In high-dimensional settings, such as large images, this generalization is achieved when a sufficiently large and diverse training dataset is made available.

FIG. 7 is a flow chart for a method 700 of determining surface quality scores associated with a 3D surface of a dental site using a trained machine learning model, in accordance with embodiments of the present disclosure. At block 702, processing logic inputs data from one or more intraoral scans (e.g., points from one or more intraoral scans and/or the intraoral scans themselves) into one or more trained machine learning models. At block 704, processing logic receives an output from the trained machine learning model (e.g., such as a trained neural network) comprising quality scores for each of the points of the one or more input intraoral scans.

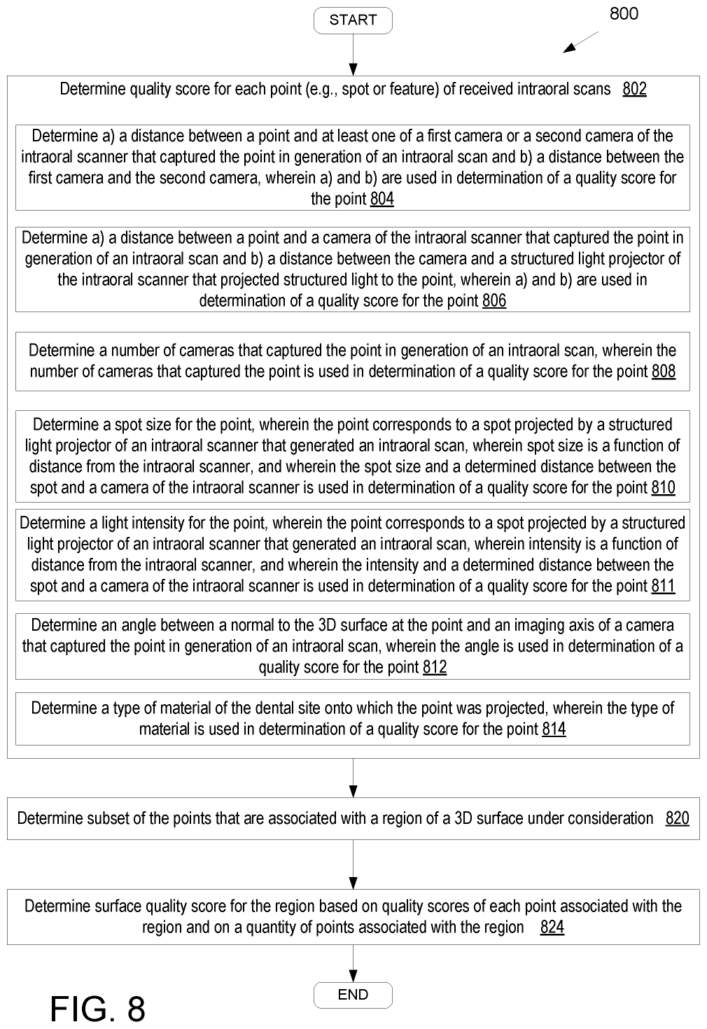

FIG. 8 is a flow chart for a method 800 of determining surface quality scores associated with a 3D surface of a dental site, in accordance with embodiments of the present disclosure. Method 800 includes at block 802 determining a quality score for each point of received intraoral scans. A point of an intraoral scan may correspond to a point (e.g., spot or feature) of structured light that was projected onto a dental site and was captured by one or more camera of an intraoral scanner. A point of an intraoral scan may correspond to an x,y,z coordinate as measured by a camera or other optical sensor of an intraoral scanner, where z may represent distance from the intraoral scanner. A quality score for a point may represent an error and/or confidence associated with that point. Numerous different criteria may be used to score points, some of which are set forth herein.

In some embodiments, the intraoral scanner includes multiple cameras, where some or all of the cameras may capture a point in generation of an intraoral scan. In one embodiment, to determine a quality score for a point in an intraoral scan and/or in a 3D surface at block 804 processing logic determines a distance between the point and the intraoral scanner (e.g., at least one of a first camera or a second camera of the intraoral scanner that captured the point in generation of the intraoral scan). Processing logic may additionally determine a distance between the first camera that captured the point and the second camera that also captured the point and/or one or more additional cameras that may have also captured the point. In embodiments, the maximum distance between any two cameras that captured the point is determined. Processing logic may determine a triangulation angle for the point based on the distance to the point and the distance between the cameras that captured the point. The lower the distance to the point and the greater the distance between the cameras, the greater the triangulation angle and thus the greater the accuracy of the determined distance to the point. Accordingly, the quality score may be directly proportional to the distance between the cameras and may be inversely proportional to the distance to the point. In one embodiment, an error for the determined distance to the point is determined using the function e=z²/bf, where e is an error for the determined distance to the point, z is the determined distance to the point, f is the focal length of the cameras, and b is the base (the distance between the two cameras). In embodiments, the error associated with the triangulation angle of the point is determined, and the error is used to compute the quality score for the point, wherein the quality score is inversely proportional to the error.

In some embodiments, the intraoral scanner includes one or more structured light projector that projects a pattern of structured light onto a dental surface, and one or more cameras that capture images of the structured light on the dental surface. In one embodiment, to determine a quality score for a point in an intraoral scan and/or in a 3D surface at block 806 processing logic determines a distance between the point and the intraoral scanner (e.g., a camera of the intraoral scanner that captured the point in generation of the intraoral scan). Processing logic may additionally determine a distance between the camera that captured the point and a structured light projector that projected the light for the point onto the dental site. Processing logic may determine a triangulation angle for the point based on the distance to the point and the distance between the camera and the structured light projector. The lower the distance to the point and the greater the distance between the camera and the structured light projector, the greater the triangulation angle and thus the greater the accuracy of the determined distance to the point. Accordingly, the quality score may be directly proportional to the distance between the camera and the structured light projector and may be inversely proportional to the distance to the point. In one embodiment, an error for the determined distance to the point is determined using the function e=z²/bf, where e is an error for the determined distance to the point, z is the determined distance to the point, f is the focal length of the camera, and b is the base (the distance between the camera and the structured light projector). In embodiments, the error associated with the triangulation angle of the point is determined, and the error is used to compute the quality score for the point, wherein the quality score is inversely proportional to the error.

In some embodiments, the intraoral scanner includes multiple cameras, where some or all of the cameras may capture a point in generation of an intraoral scan. The greater the number of cameras that captured the point and that agree or approximately agree on the coordinates of the point, the greater the confidence for the point and the lower the error associated with the point. In one embodiment, to determine a quality score for a point in an intraoral scan and/or in a 3D surface at block 808 processing logic determines a number of cameras that captured the point. The quality score for the point may be directly proportional to the number of cameras that captured the point, where the quality score increases with an increase in the number of cameras that captured the point.

In some embodiments, structured light projectors project structured/patterned light comprising spots and/or other shapes/features onto a dental site. In embodiments, spot size is a function of distance from the intraoral scanner. The size of a spot or other feature generally increases with distance from the intraoral scanner. Processing logic may include calibration data indicating the approximate spot/feature size that should be detected at various distances. For example, spots projected onto a surface at a first relatively close distance may have a first general spot size, spots projected onto a surface at a second distance that is greater than the first distance may have a second larger general spot size, and spots projected onto a surface at a third distance that is greater than the second distance may have a third even larger general spot size. At block 810 processing logic may predict a spot size for a spot projected onto a point at a dental site based on a determined distance for that point. Processing logic may measure a spot size at the point, and then compare the measured spot size for the projected spot at the point to the predicted spot size. The quality score may be determined based on a difference between the estimated spot size and the measured spot size. The quality score may be indirectly proportional to the difference. Accordingly, as the difference increases, the quality score for the point may decrease.

In some embodiments, structured light projectors project structured/patterned light comprising spots and/or other shapes/features (e.g., such as a checkerboard pattern comprising checkerboard features) onto a dental site. In embodiments, an intensity of a spot or other projected feature is a function of distance from the intraoral scanner. The intensity of a spot or other feature generally decreases with distance from the intraoral scanner. Processing logic may include calibration data indicating the approximate spot/feature intensity that should be detected at various distances. For example, spots projected onto a surface at a first relatively close distance may have a first general intensity, spots projected onto a surface at a second distance that is greater than the first distance may have a second lower intensity, and spots projected onto a surface at a third distance that is greater than the second distance may have a third even lower intensity. At block 811 processing logic may predict an intensity for a spot/feature projected onto a point at a dental site based on a determined distance for that point. Processing logic may measure an intensity at the point, and then compare the measured intensity for the projected spot/feature at the point to the predicted intensity. The quality score may be determined based on a difference between the estimated intensity and the measured intensity. The quality score may be indirectly proportional to the difference. Accordingly, as the difference increases, the quality score for the point may decrease.

In some embodiments, the accuracy of a distance coordinate for a point varies based on an angle of a surface at the point to a camera that captured an image of the point and/or to a light source that projected structured light onto the point. At block 812, processing logic may determine an angle between a normal to the 3D surface at the point and an imaging axis of a camera that captured the point in generation of an intraoral scan. The angle may then be used in determination of a quality score for the point. As the angle increases, the quality score for the point decreases.

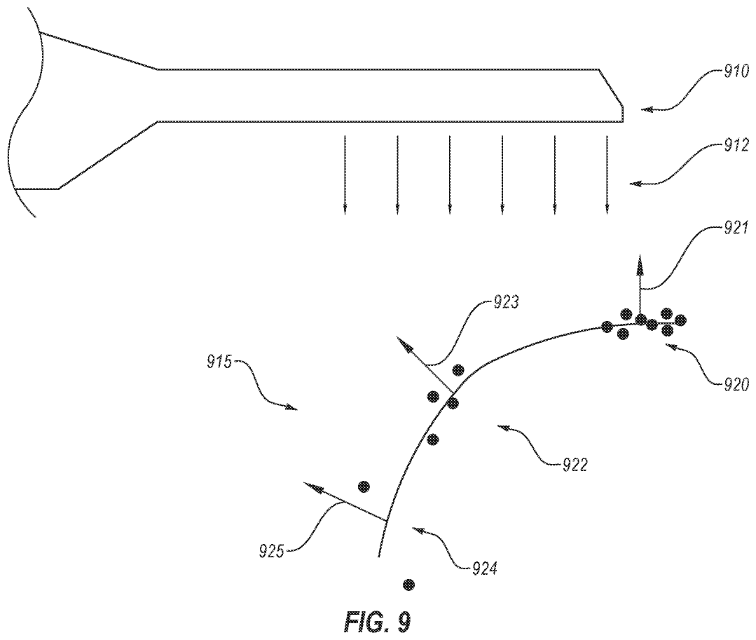

FIG. 9 illustrates scoring of regions of a dental site based on angle, in accordance with embodiments of the present disclosure. FIG. 9 shows an intraoral scanner 910 (e.g., which may correspond to intraoral scanner 150 of FIG. 1), and rays 912 corresponding roughly to an imaging axis of one or more cameras of the intraoral scanner 910. FIG. 9 further shows a dental site 915 being scanned. A first normal 921 to a surface of dental site 915 at a first region associated with a first set of points 920 is approximately parallel to the rays 912. Accordingly, the first set of points will have high accuracy, and the quality score associated with the first set of points will be relatively high. A second normal 923 to a surface of dental site 915 at a second region associated with a second set of points 922 is at approximately a 45 degree angle to the rays 912. Accordingly, the second set of points will have a medium accuracy, and the quality score associated with the second set of points 922 will be lower than the quality score of the first set of points 920. A third normal 925 to a surface of dental site 915 at a third region associated with a third set of points 924 is at approximately a 90 degree angle to the rays 912. Accordingly, the third set of points will have a low accuracy, and the quality score associated with the third set of points 924 will be lower than the quality score of the second set of points 920.

Returning to FIG. 8, in some embodiments the type of material for the dental site may affect the accuracy of captured points. For example, it may be more difficult to generate accurate scans of highly reflective objects such as objects composed of titanium, which may include certain types of restorative objects (e.g., dental implants, studs, etc.). Accordingly, in some embodiments at block 814 processing logic determines a type of material a dental site is made out of and determines a quality score for a point based at least in part on the material of the dental site associated with the point. In some embodiments, a user inputs a type of material. In some embodiments, intraoral scans and optionally associated 2D images such as color images and/or NIR images are input into a trained machine learning model that classifies dental sites/objects according to material. The trained machine learning model may be, for example, a convolutional neural network as described above.

Other parameters or metrics may also be determined for a point and may be used to compute a quality score for the point. For example, an intraoral scanner may have lower accuracy when capturing points outside of a distance range from the scanner. Accordingly, points that are too close to the scanner or too far away from the scanner (i.e., outside of the optimal scanner distance) may yield lower accuracy and may thus be scored with lower quality scores than points that are within the optimal scanner distance. Additionally, if a scanner is moving too quickly during scanning, than generated scans may be blurry, which may result in lower quality points. Additionally, if a dental site is partially obscured by saliva and/or blood, this may result in measured points that are not actually part of the dental site surface. Accordingly, in embodiments saliva and/or blood detection may be performed, such as by processing intraoral scans using a trained machine learning model (e.g., a neural network), which may classify regions as teeth, blood and/or saliva. Points at regions classified as blood and/or saliva may have lower surface quality scores than points at regions classified as teeth. In one embodiment, blood and/or saliva is detected as described in U.S. application Ser. No. 16/809,451, filed Mar. 4, 2020, which is incorporated by reference herein in its entirety.

In some embodiments, multiple parameters or metrics are determined for a point, such as the parameters determined at blocks 804-814 (e.g., parameters/metrics such as triangulation angle to the point, number of cameras that captured the point, difference between estimated spot size and measured spot size, difference between estimated intensity and measured intensity, angle of surface at the point relative to the intraoral scanner imaging axis, type of material, and so on). Each of these parameters may provide clues as to the accuracy of the measured location of the point (e.g., x, y and/or z position). In embodiments, some or all of these parameters (and optionally other parameters) are used to compute a quality score for the point. In embodiments, some or all of these parameters are combined into a function to estimate a point quality statistically. Some of the mentioned parameters may have a greater impact on accuracy of the measured location for the point. Accordingly, some parameters may be weighted more heavily than other parameters in determination of a quality score for the point. In some embodiments, quality scores are determined using some or all of the techniques discussed herein (e.g., at blocks 804-814), and a weighted or unweighted average of the quality scores is computed to result in a final quality score for the point.

In some embodiments, once some or all of the above described parameters are determined for one or more points of an intraoral scan and/or a region of a 3D surface, a regression analysis may be used on the one or more points and their associated parameters to determine final quality scores for the one or more points. In some embodiments, once some or all of the above described parameters are determined for one or more points of an intraoral scan and/or a region of a 3D surface, the data for the one or more points may be input into a trained machine learning model such as a neural network, which may output final quality scores for the one or more points. In one embodiment, the regression analysis and/or machine learning model operates on a single point at a time to output a quality score for that point. In one embodiment, the regression analysis and/or machine learning model operates on multiple points in parallel to determine quality scores for the multiple points, where parameters (e.g., x,y,z position, distance, triangulation angle, surface angle, number of cameras capturing the point, etc.) for one point may affect not only the quality score for that point but also the quality scores for other nearby points.

At block 820, processing logic may determine a region of a 3D surface (e.g., a 1×1 mm square region or a 1×1×1 mm cubic region) and determine a subset of points that are associated with the region. At block 824, processing logic determines a surface quality score for the region based on the quality scores of the points included in the region and on a quantity of the points included in the region. A surface quality of a region may be a function of the number of points in the region and the error level of each of those points. For example, a surface quality score for a 1 mm×1 mm×1 mm voxel region can be estimated by computing:

$$E = \frac{1}{\sqrt{{\sum }_{i} \frac{1}{{e}_{i}^{2}}}}$$

which is the standard error of the weighted mean, and where E is the surface quality score representing error of the region, i is a given point, and e is the quality score representing error of the given point.

As the quality scores of the points in the region increase, the surface quality score for the region also increases. Additionally, as the quantity and/or density of points at the region (which correlates to a resolution of the region) goes up, the surface quality score for that region also goes up. The surface quality score for a region may also depend on a surface roughness of the region in some embodiments. The 3D surface at the region may be determined by determining a surface that is an average of the multiple points. The 3D surface may or may not correspond to actual positions of points. The surface roughness for a region may be computed by determining distances between points in the region and the closest respective location on the 3D surface. The greater the distances, the higher the roughness and the lower the surface quality score. The roughness may be computed as an average of the distances between the points in the region and the closest respective locations on the 3D surface in the region in embodiments.

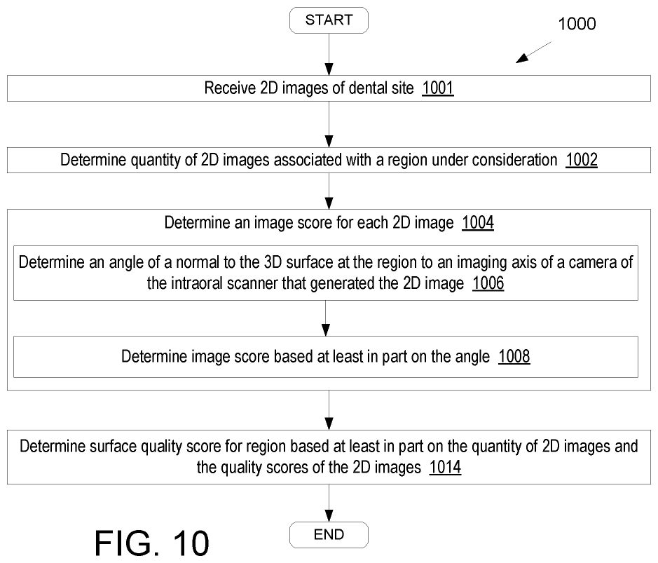

FIG. 10 is a flow chart for a method 1000 of determining surface quality scores for 2D images, in accordance with embodiments of the present disclosure. At block 1001 of method 1000, processing logic receives 2D images of a dental site. The 2D images may include color images and/or NIR images in embodiments. The intraoral scanner may interleave generation of intraoral scans with generation of 2D images. Accordingly, as intraoral scans are generated 2D images may also be generated.

At block 1002, processing logic determines a quantity of 2D images associated with a region of a 3D surface under consideration. The greater the number of 2D images, the more data that there may be available for the region and the higher the ultimate surface quality score for that region in embodiments.

At block 1004, processing logic determines an image score for each 2D image. In one embodiment, at block 1006 processing logic determines an angle of an imaging axis of the intraoral scanner to a normal to the 3D surface at the region. At block 1008, processing logic may determine an image score for the image based at least in part on the angle. The greater the angle, the lower the image score in some embodiments, similar to the relationship between quality scores and angle shown in FIG. 9.

At block 1014, processing logic may determine a surface quality score for a region of the 3D surface based at least in part on the quantity of 2D images associated with the region and the quality scores of the 2D images associated with the region. In some embodiments, the surface quality score determined at block 1014 may be combined with the surface quality score determined at block 824 of method 800 and/or the surface quality score determined at block 606 of method 600, such as with a weighted or unweighted average, to yield a final surface quality score for the region.

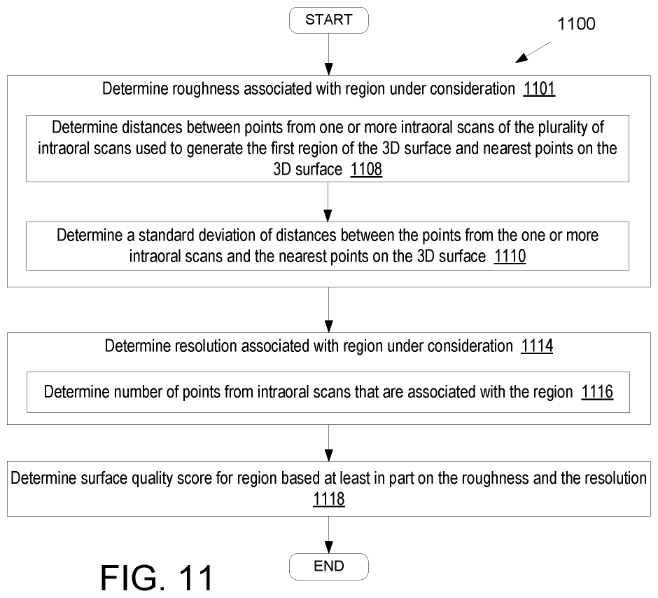

FIG. 11 is a flow chart for a method 1100 of determining surface quality scores for a region under consideration based on roughness and/or resolution, in accordance with embodiments of the present disclosure. In embodiments, a 3D surface quality can be characterized using roughness and resolution, where roughness may be a measure of a noise level of the 3D surface and resolution is related to an ability to show details of the 3D surface. For a given number of points (e.g., each having x,y,z coordinates) and a noise level, processing logic can reduce a roughness by performing a smoothing operation on the 3D surface. However, performing the smoothing operation reduces resolution in embodiments. In some embodiments, for a given scanned point or region a value computed by multiplying roughness by resolution is approximately constant. Accordingly, increasing resolution may decrease roughness and increasing roughness may decrease resolution, but the result of multiplying these two values for a given region should remain approximately constant. In some embodiments, processing logic may automatically choose per region a balance between roughness and resolution to use for that region.

At block 1101 of method 1100, processing logic determines roughness associated with a region under consideration. In one embodiment, the roughness of the region is determined as set forth in blocks 1108-1110. In one embodiment, at block 1108 processing logic determines distances between points from one or more intraoral scans associated with a region of the 3D surface and nearest points on the 3D surface. At block 1110, processing logic determines a standard deviation of distances between the points from the one or more intraoral scans and the nearest points on the 3D surface.

At block 1114, processing logic determines a resolution associated with the region under consideration. In one embodiment, the resolution is determined by determining a number of points from intraoral scans that are associated with the region. The greater the number of points in the region, the greater the resolution of the region.

At block 1118, processing logic determines a surface quality score for the region under consideration based at least in part on the roughness and the resolution. The surface quality score may be directly proportional to the resolution, such that the surface quality score increases with an increase in the resolution. Additionally, the surface quality score may be inversely proportional to the roughness, such that the surface quality score decreases with increases in roughness. In some embodiments, the surface quality score determined at block 1118 may be combined with the surface quality score determined at block 1014 of method 1000, the surface quality score determined at block 824 of method 800 and/or the surface quality score determined at block 606 of method 600, such as with a weighted or unweighted average, to yield a final surface quality score for the region.

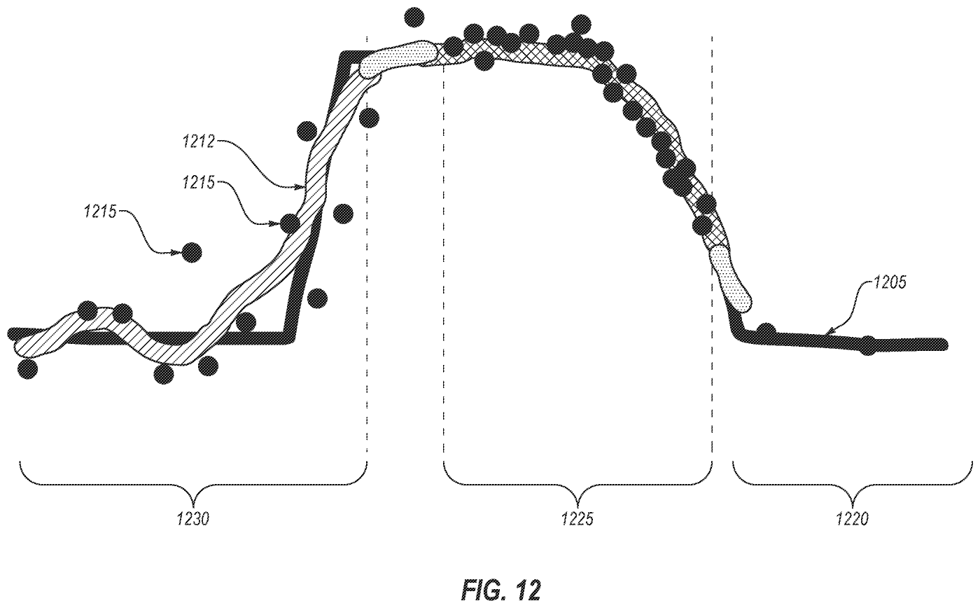

FIG. 12 illustrates scoring of regions of a dental site based on roughness and/or point density, in accordance with embodiments of the present disclosure. In FIG. 12 line 1205 represents an original dental surface, line 1212 represents a generated 3D surface created based on intraoral scans of the original dental surface, and points 1215 are points captured in intraoral scans. A first region 1220 of the 3D surface has too few data points, and is considered a hole or void in the surface. Accordingly, line 1212 does not extend into first region 1220. A second region 1225 of the 3D surface has a high smoothness and a high resolution due to a large number of points 1215 that lie closely along line 1212, and so has a high surface quality score. A third region 1230 of the 3D surface has a low to medium number of points 1215 and a high roughness due to points 1215 in third region 1230 being generally distant from line 1212, and so has a low surface quality score.

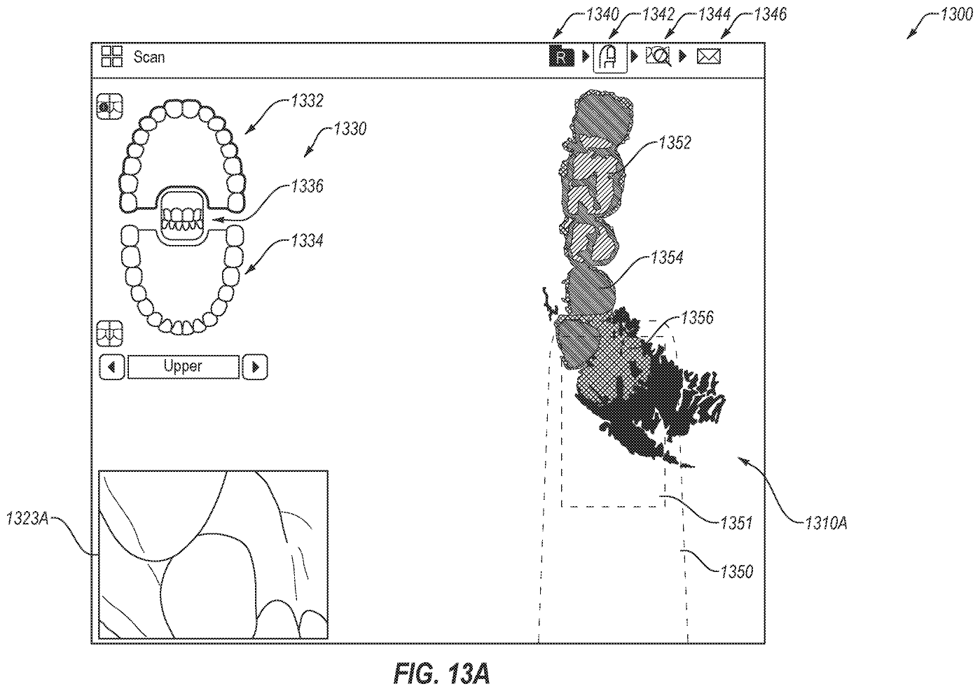

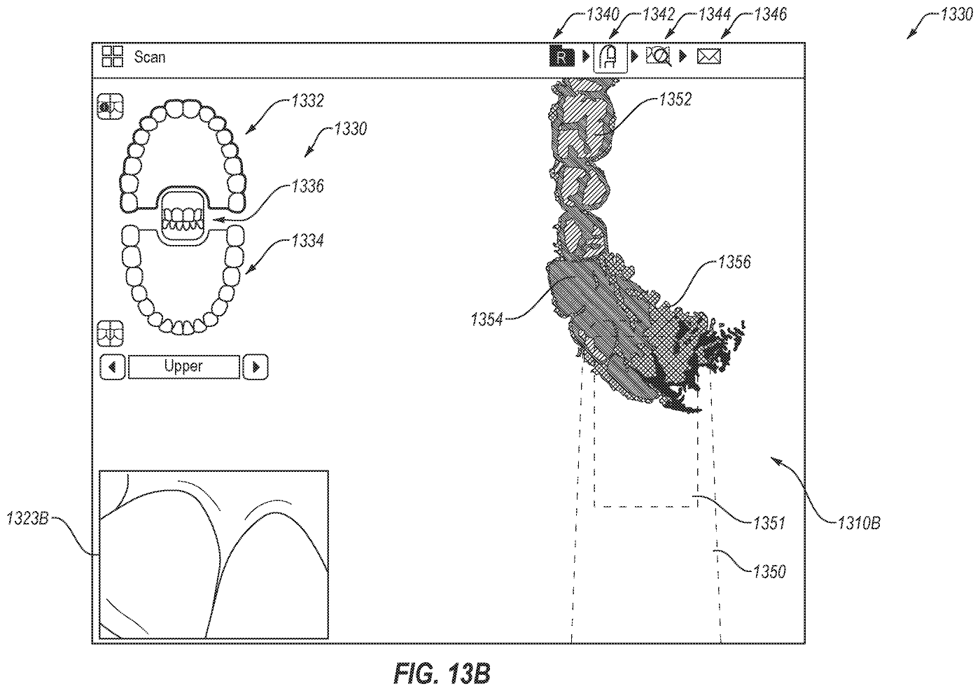

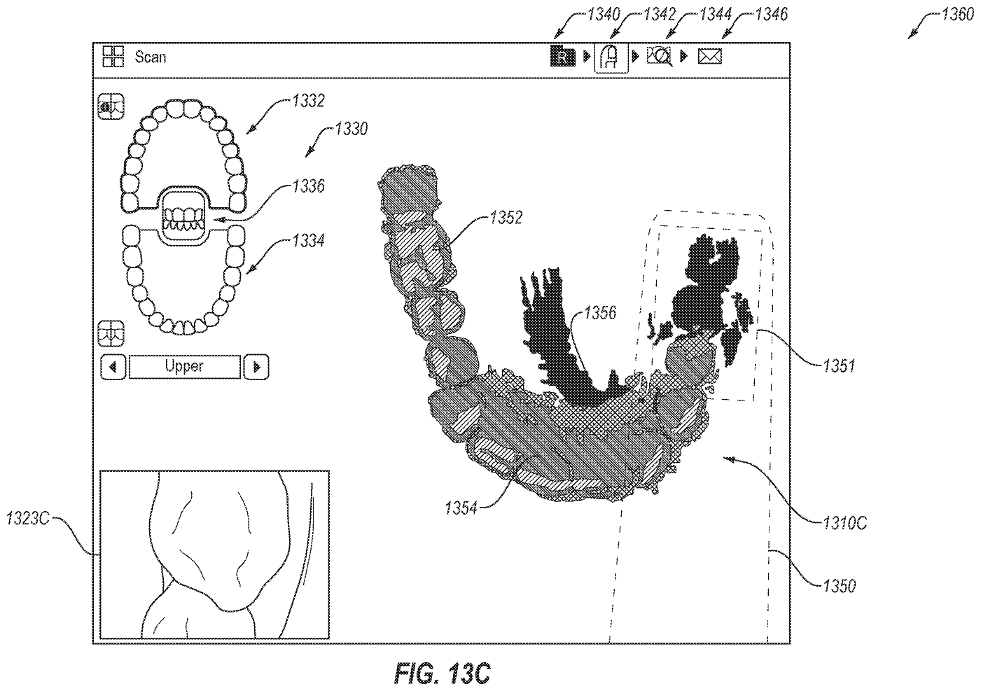

FIGS. 13A-C illustrate views of a graphical user interface of an intraoral scan application that includes a 3D surface with surface quality feedback, in accordance with embodiments of the present disclosure. FIG. 13A illustrates a first view of a graphical user interface 1300 for an intraoral scan application that includes a first 3D surface 1310A and a first 2D image 1323A of a current field of view (FOV) of an intraoral scanner, in accordance with embodiments of the present disclosure. Additionally, an outline 1350 of the intraoral scanner, including an outline 1351 of a field of view of the intraoral scanner may be shown relative to the 3D surface 1310A. FIG. 13B illustrates a second view of a graphical user interface 1330 for the intraoral scan application that includes a second 3D surface 1310B and a second 2D image 1323B of the current FOV of the intraoral scanner. Additionally, an outline 1350 of the intraoral scanner, including an outline 1351 of a field of view of the intraoral scanner may be shown relative to the 3D surface 1310A. FIG. 13C illustrates a third view of a graphical user interface 1360 for the intraoral scan application that includes a third 3D surface 1310C and a third 2D image 1323C of the current FOV of the intraoral scanner. Additionally, an outline 1350 of the intraoral scanner, including an outline 1351 of a field of view of the intraoral scanner may be shown relative to the 3D surface 1310A. The 3D surfaces 1310A, 1310B, 1310C are generated by registering and stitching together multiple intraoral scans captured during an intraoral scanning session. As each new intraoral scan is generated, that scan is registered to the 3D surface and then stitched to the 3D surface. Accordingly, the 3D surface becomes more and more accurate with each intraoral scan, until the 3D surface is complete. A 3D model may then be generated based on the intraoral scans. 3D surface 1310A is an initial 3D surface of the dental site shortly after scanning has begun, 3D surface 1310B is an updated version of 3D surface 1310A after further intraoral scans have been captured, and 3D surface 1310C is an updated version of 3D surface 1310B after still further intraoral scans have been captured.

During intraoral scanning, it can be challenging for a user of the intraoral scanner to determine whether they are making progress in scanning one or more regions of a dental site. This is especially true for hard to capture regions, such as back molars, teeth with deep recesses, interproximal spaces between teeth, etc. and regions for which a high accuracy is desired such as preparations. Accordingly, in embodiments processing logic continuously computes surface quality scores for multiple regions of 3D surfaces 1310A-C and determines visualizations to use for those regions to visually indicate the surface quality scores. In some embodiments, visualizations used to depict surface quality scores are based on color and/or shading. For example, green may indicate a high surface quality score, yellow may indicate a mid-level surface quality score, and red may indicate a low surface quality score.

As shown for 3D surface 1310A of FIG. 13A, first regions are depicted with a first visualization 1352 indicating a high surface quality score, second regions are depicted with a second visualization 1354 indicating a medium surface quality score, and third regions are depicted with a third visualization 1356 indicating a low surface quality score. Other visualizations that may be used instead of or in addition to color to indicate surface quality score include transparency level, flickering or blinking frequency, etc. If transparency level is used to indicate surface quality score, then a background image, color, texture, pattern, etc. may be shown behind the 3D surface 1310A-C so that the differing levels of transparency are more readily visible in some embodiments. In some embodiments, an accurate color of a scanned dental site is determined and the 3D surface is shown using the determined color of the dental site. In such embodiments, color may not be used to represent surface quality, and other visualization options such as transparency and/or flickering may instead be used to represent surface quality. One advantage of using transparency for marking surface quality is that the natural worst case for surface quality is no data at all, which correlates to 100% transparency. In embodiments, a smooth transition may be provided between no data (100% transparent) to little data (very transparent) to more data (slightly transparent) to sufficient data (fully opaque). In some embodiments, flickering may be performed for transparency (e.g., flickering between transparency levels), for color (e.g., flickering between colors), and so on. A rate of flickering may be inversely proportional to the surface quality level in embodiments.

In some embodiments, surface quality scores may be called out using flagging, such as with arrows that point to various regions. The surface quality score for a region may be reflected in properties of the arrow or other flag pointing to and/or otherwise calling out the region. For example, a thickness of an arrow (e.g., where increased thickness represents a lower surface quality score), a length of an arrow (e.g., where increased length represents a lower surface quality score), a flashing rate of an arrow (e.g., where an increased flashing rate represents a lower surface quality score), and so an may be based on a surface quality score of a region to which the arrow points. In some embodiments, flagging and/or arrows for regions may be visible even in instances where the region itself is not visible, such as if the region is obscured by other surfaces in the current view of the 3D surface. In one embodiment, flagging of a region with a flag that indicates surface quality score is provided as set forth in U.S. Pat. No. 9,510,757, issued Dec. 6, 2016, which is incorporated by reference herein in its entirety.

3D surface 1310B of FIG. 13B shows an updated 3D surface after additional intraoral scans have been registered and stitched to 3D surface 1310A. Accordingly, regions that were shown with second visualization 1354 in 3D surface 1310A may be shown with the first visualization 1352 in 3D surface 1310B. Additionally, regions that were shown with third visualization 1356 in 3D surface 1310A may be shown with the second visualization 1354 in 3D surface 1310B. Additionally, the 3D surface may have grown do to further regions of the dental site having been scanned.

3D surface 1310C of FIG. 13C shows an updated 3D surface after additional intraoral scans have been registered and stitched to 3D surface 1310B. Accordingly, regions that were shown with second visualization 1354 in 3D surface 1310B may be shown with the first visualization 1352 in 3D surface 1310C. Additionally, regions that were shown with third visualization 1356 in 3D surface 1310B may be shown with the second visualization 1354 in 3D surface 1310C. Additionally, the 3D surface may have grown do to further regions of the dental site having been scanned.

The GUI for the intraoral scan application may show the 2D image 1323A-C in a region of the GUI's display. The 2D images may be generated at a frame rate of about 20 frames per second (updated every 50 milliseconds) to about 15 frames per second (updated every 66 milliseconds) in embodiments.

In one embodiment, as shown, a scan segment indicator 1330 may include an upper dental arch segment indicator 1332, a lower dental arch segment indicator 1334 and a bite segment indicator 1336. While the upper dental arch is being scanned, the upper dental arch segment indicator 1332 may be active (e.g., highlighted). Similarly, while the lower dental arch is being scanned, the lower dental arch segment indicator 1334 may be active, and while a patient bite is being scanned, the bite segment indicator 1336 may be active. A user may select a particular segment indicator 1332, 1334, 1336 to cause a 3D surface associated with a selected segment to be displayed. A user may also select a particular segment indicator 1332, 1334, 1336 to indicate that scanning of that particular segment is to be performed. Alternatively, processing logic may automatically determine a segment being scanned, and may automatically select that segment to make it active.

The GUI of the intraoral scan application may further include a task bar with multiple modes of operation or phases of intraoral scanning. Selection of a patient selection mode 1340 may enable a doctor to input patient information and/or select a patient already entered into the system. Selection of a scanning mode 1342 enables intraoral scanning of the patient's oral cavity. After scanning is complete, selection of a post processing mode 1344 may prompt the intraoral scan application to generate one or more 3D models based on intraoral scans and/or 2D images generated during intraoral scanning, and to optionally perform an analysis of the 3D model(s). Examples of analyses that may be performed include analyses to detect areas of interest, to assess a quality of the 3D model(s), and so on. Once the doctor is satisfied with the 3D models, they may generate orthodontic and/or prosthodontic prescriptions. Selection of an prescription fulfillment mode 1346 may cause the generated orthodontic and/or prosthodontic prescriptions to be sent to a lab or other facility to cause a prosthodontic device (e.g., a crown, bridge, denture, etc.) or orthodontic device (e.g., an orthodontic aligner) to be generated.

FIGS. 13A-13C show a single 3D surface in which various visualizations are used to depict surface quality scores. In some embodiments, multiple copies of a 3D surface (or regions of a 3D surface) are output to the display in parallel (e.g., are shown together in the GUI). For example, one instance of a 3D surface may be monochrome or depict color information of the scanned dental site, and another instance of the 3D surface may include visualizations showing surface quality scores. In some embodiments, the second instance of the 3D surface is shown when there are one or more regions having surface quality scores that are below a surface quality threshold. In some embodiments, the second instance of the 3D surface having visualizations that indicate surface quality scores is depicted with a smaller size and/or zoom setting than the first instance of the 3D surface. In one embodiment, a user may select or click on the instance of the 3D surface having visualizations that indicate surface quality scores to cause that instance of the 3D surface to enlarge (e.g., to become a primary or main instance of the 3D surface). This may cause the arrangement of the first and second instances of the 3D surface in the display to change as well. In some embodiments, more than two instances of the 3D surface are shown. For example, a first instance of the 3D surface having coloration that reflects colors of the dental arch, a second instance of the 3D surface that lacks color or that is all the same color, and a third instance of the 3D surface that shows surface quality may be output to the display in parallel. At any given time one of the multiple instances of the 3D surface may be a main or primary instance that is larger then the other instances of the 3D surface. At any time a doctor or other user may select one of the other instances of the 3D surface to cause the selected instance to become the main or primary instance (and to thus enlarge and/or be moved to a different location on the display).

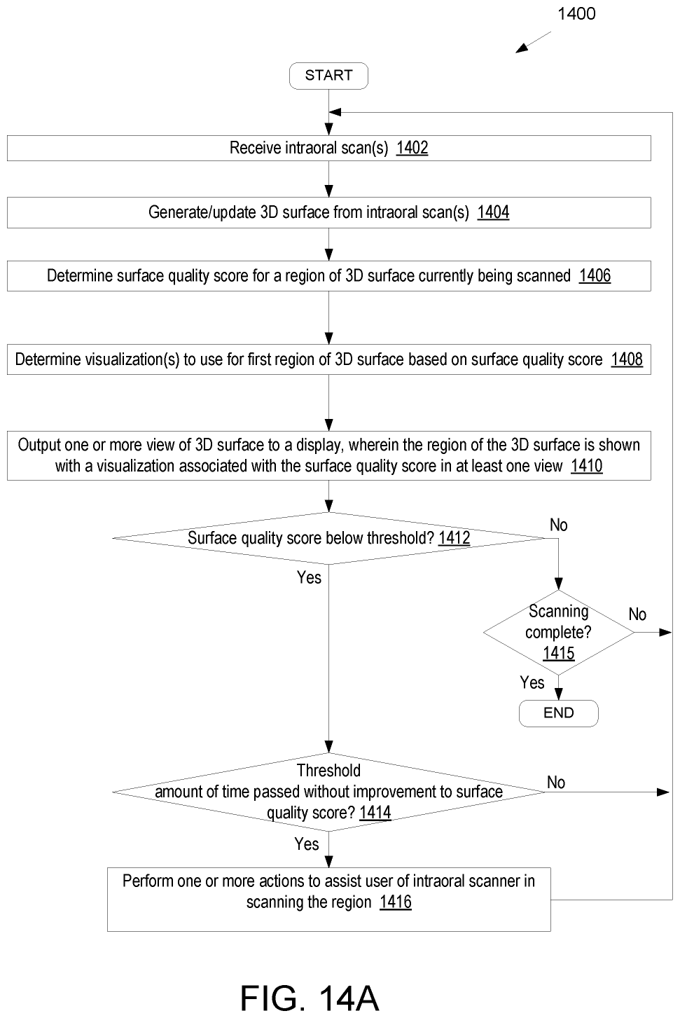

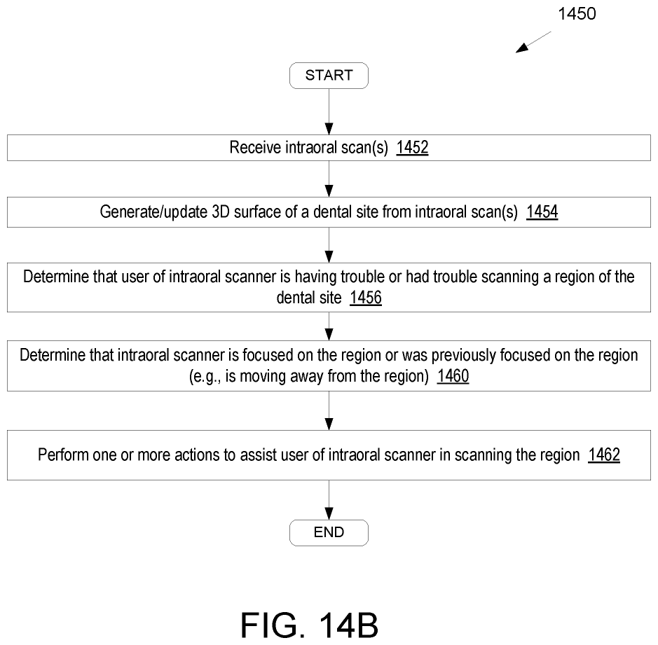

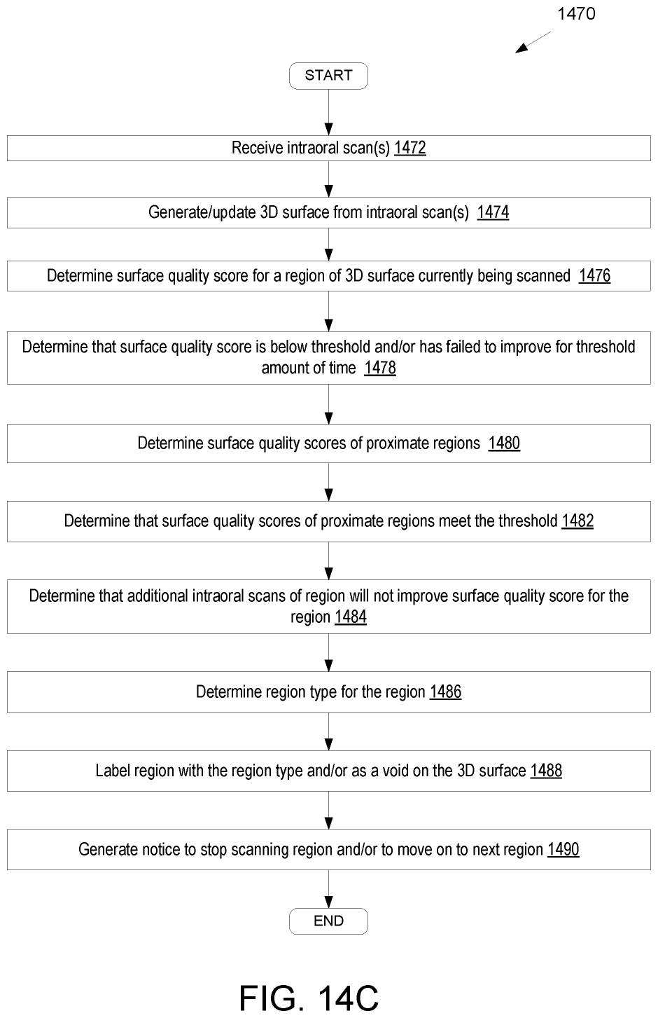

FIG. 14A is a flow chart for a method 1400 of providing user assistance during intraoral scanning, in accordance with embodiments of the present disclosure. At block 1402 of method 1400, processing logic receives one or more intraoral scans and/or 2D images of a dental site. Processing logic may additionally receive one or more two-dimensional (2D) images of the dental site, which may include color 2D images, near infrared (NIR or NIRI) 2D images, 2D images generated under ultraviolet light, and so on. Each of the intraoral scans may include three-dimensional information about a captured portion of the dental site. For example, each intraoral scan may include point clouds. In embodiments, each intraoral scan includes three dimensional information (e.g., x,y,z coordinates) for multiple points on a dental surface. Each of the multiple points may correspond to a spot or feature of structured light that was projected by a structured light projector of the intraoral scanner onto the dental site and that was captured in images generated by one or more cameras of the intraoral scanner.

At block 1404, processing logic generates a 3D surface representing the scanned dental site using the one or more received intraoral scans. This may include registering and stitching together multiple intraoral scans and/or registering and stitching one or more intraoral scans to an already generated 3D surface to update the 3D surface. The registration and stitching process may be performed as described in greater detail above. As further intraoral scans are received, those intraoral scans may be registered and stitched to the 3D surface to add information for more regions/portions of the 3D surface and/or to improve the quality of one or more regions/portions of the 3D surface that are already present. In some embodiments, the generated surface is an approximated surface that may be of lower quality than a surface that will be later calculated.

At block 1406, processing logic determines surface quality scores for one or more regions of the 3D surface. In some embodiments, the 3D surface is divided into a plurality of regions, which may have a same size or different sizes. For example, in one embodiment the 3D surface is divided into approximately mm sized regions (e.g., 1 mm square region, 1 mm diameter region, etc.). Alternatively, larger or smaller regions may be used. Surface quality scores may be determined for each region.

At block 1408, processing logic determines one or more visualizations to use for the various regions of the 3D surface based on surface quality scores assigned to those regions.

At block 1410, processing logic outputs one or more views of the 3D surface to a display. Each region of the 3D surface may be displayed with a respective visualization that was determined for that region based on a surface quality score of the region. Accordingly, the first region may be shown with a visualization associated with the surface quality score for the first region in the one or more view.

At block 1412, processing logic determines whether a surface quality score for one or more regions is below a surface quality threshold. This may include determining one or more surface quality thresholds to apply for the one or more regions based on a classification of the regions. For example, data associated with a region (e.g., from intraoral scans, the 3D surface, 2D images, etc.) may be input into a trained machine learning model that outputs a dental object classification for the region. Different dental object classifications may be associated with different thresholds. In one embodiment, a rubric is determined based on the dental object classification for the region, where the rubric includes a surface quality threshold to apply. If the surface quality score for the region(s) is below the determined surface quality threshold, the method continues to block 1414. If the surface quality score for the region(s) is at or above the surface quality threshold, the method may continue to block 1415. In one embodiment, a region under consideration corresponds to a region of a dental site that is currently in a field of view of the intraoral scanner. In one embodiment, a region under consideration corresponds to a region of the dental site that was recently within the field of view of the intraoral scanner (e.g., was in the field of view less than a threshold amount of time prior to present).

At block 1415, processing logic determines whether scanning is complete. If scanning is not complete (e.g., more intraoral scans are still being generated), the method returns to block 1402 and additional intraoral scans are received and processed. This may result in updates to the 3D surface at block 1404 and possibly to changes in the surface quality scores for one or more regions of the 3D surface at block 1406. If scanning is complete, then the method may end.

At block 1414, processing logic may determine whether a threshold amount of time has passed without improvement to the surface quality score or without at least a threshold amount of improvement to the surface quality score. Alternatively, or additionally, processing logic may determine whether a velocity of the intraoral scanner is below a velocity threshold. Additionally, or alternatively, processing logic may determine whether the intraoral scanner has begun to move away from the region under consideration without the surface quality score reaching the threshold surface quality. In embodiments, if one or more of these conditions are met, the method continues to block 1416. If none of these conditions are met, then the method may return to block 1402.

At block 1416, processing logic performs one or more actions to assist a user of the intraoral scanner in scanning of the region under consideration and/or to alert the user that it may not be possible to successfully scan the region under consideration. In some embodiments, at block 1416 processing logic determines that additional intraoral scans of the first region will not improve the surface quality score for the region. Different types of actions may be performed to assist the user. In some embodiments, processing logic automatically increases a zoom setting to zoom in on the region under consideration to make it easier for the user to view the region of the 3D surface.

In some embodiments, processing logic generates a notice for a user to stop generating new intraoral scans of the region and/or to move on to a next region. In some embodiments, processing logic labels the region as a void.

In some embodiments, processing logic generates an overlay and outputs the overlay over the 3D surface and/or a 2D image of a current FOV of the intraoral scanner to show where/how the intraoral scanner should be positioned and/or oriented relative to where/how the intraoral scanner is currently positioned and/or oriented. In some embodiments, processing logic generates an overlay showing a path for the intraoral scanner to follow to capture intraoral scans that will improve the surface quality for the region. In some embodiments, processing logic determines a classification for the region (e.g., using a trained machine learning model such as a neural network), and determines one or more suggestions for the doctor and/or patient to perform to improve a quality of the captured intraoral scans of the region based at least in part on the classification for the region.

In some embodiments, processing logic determines one or more problems with the region of the 3D surface (e.g., obscured by lips, obscured by gums, obscured by blood and/or saliva, obscured by collapsed gum over region, bad angle, etc.) and outputs one or more suggestions for the patient and/or doctor to perform to improve a quality of the captured intraoral scans of the region. The problems may be determined by inputting data associated with the 3D surface (e.g., intraoral scans, 2D images, the 3D surface itself, a projection of the 3D surface, etc.) into a trained machine learning model that may output indications of one or more detected problems.

In some embodiments, the intraoral scanner includes multiple cameras each having a different position and/or orientation in a head of the intraoral scanner (and thus a different field of view). Due to the different positions/orientations of the various cameras, some cameras may be better able to capture information for a region or a part of a region. Each of the cameras may generate 2D images, any of which may be used as a viewfinder image to show a current field of view of the intraoral scanner. Processing logic may select a camera that is most capable of capturing high quality data for the region, and may show images generated by the selected camera. This may cause the user to adjust a position/orientation of the intraoral scanner so that they can better see the region as captured by the selected camera. This may improve a quality of intraoral scans of the region in embodiments. Any one or more of these actions and/or other actions may be performed in embodiments to improve the quality of the 3D surface.

In some embodiments, processing logic automatically adjusts one or more algorithms used for processing intraoral scan data associated with the region. In one embodiment, processing logic determines one or more algorithms to use for processing intraoral scan data (e.g., intraoral scans, 2D images, regions of 3D surfaces, etc.). Examples of algorithms include registration algorithms, stitching algorithms, moving tissue detection and removal algorithms, object detection algorithms, soft tissue detection algorithms, and so on.

FIG. 14B is a flow chart for a method 1450 of providing user assistance during intraoral scanning, in accordance with embodiments of the present disclosure. In embodiments, method 1450 operates together with method 1400 to provide user assistance during scanning. At block 1452 of method 1450, processing logic receives one or more intraoral scans and/or 2D images of a dental site. Processing logic may additionally receive one or more two-dimensional (2D) images of the dental site, which may include color 2D images, near infrared (NIR or NIRI) 2D images, 2D images generated under ultraviolet light, and so on.

At block 1454, processing logic generates a 3D surface representing the scanned dental site using the one or more received intraoral scans.

At block 1456, processing logic determines that a user is having trouble scanning a region of the dental site or had trouble scanning the region of the dental site (e.g., if the intraoral scanner is moving away from the region or is now scanning a different region). Multiple different techniques may be used to determine whether the user is having or had trouble scanning the region of the dental site. In one embodiment, if the intraoral scanner remains focused or remained focused on the region of the dental site for at least a threshold amount of time, then processing logic may determine that the user is having trouble or had trouble scanning the region. In one embodiment, a surface quality score for the region may be computed, and if the surface quality score is below a threshold value after a threshold amount of time, then processing logic may determine that the user is having trouble or had trouble scanning the region. In one embodiment, if a velocity of the intraoral scanner is below a threshold velocity and is focused on the region, then processing logic may determine that the user is having trouble scanning the region.

At block 1460, processing logic determines that the intraoral scanner is focused on the region or was previously focused on the region (e.g., is moving away from the region). This may be determined by comparing a current field of view to the region to determine if there is a threshold amount of overlap between the current field of view and the region (e.g., by comparing a most recent intraoral scan to the region of the 3D surface).

At block 1462, processing logic performs one or more actions to assist the user of the intraoral scanner in scanning the region. Any of the previously described actions that provide user assistance may be performed. In some embodiments, user assistance is not initiated until the user starts moving the intraoral scanner away from the region.

FIG. 14C is a flow chart for a method 1470 of providing user assistance during intraoral scanning in instances where surface quality of a region is not improving, in accordance with embodiments of the present disclosure. At block 1472 of method 1470, processing logic receives one or more intraoral scans and/or 2D images of a dental site. Processing logic may additionally receive one or more two-dimensional (2D) images of the dental site, which may include color 2D images, near infrared (NIR or NIRI) 2D images, 2D images generated under ultraviolet light, and so on. Each of the intraoral scans may include three-dimensional information about a captured portion of the dental site. For example, each intraoral scan may include point clouds. In embodiments, each intraoral scan includes three dimensional information (e.g., x,y,z coordinates) for multiple points on a dental surface. Each of the multiple points may correspond to a spot or feature of structured light that was projected by a structured light projector of the intraoral scanner onto the dental site and that was captured in images generated by one or more cameras of the intraoral scanner.

At block 1474, processing logic generates a 3D surface representing the scanned dental site using the one or more received intraoral scans. This may include registering and stitching together multiple intraoral scans and/or registering and stitching one or more intraoral scans to an already generated 3D surface to update the 3D surface. The registration and stitching process may be performed as described in greater detail above. As further intraoral scans are received, those intraoral scans may be registered and stitched to the 3D surface to add information for more regions/portions of the 3D surface and/or to improve the quality of one or more regions/portions of the 3D surface that are already present. In some embodiments, the generated surface is an approximated surface that may be of lower quality than a surface that will be later calculated.

At block 1476, processing logic determines surface quality scores for one or more regions of the 3D surface. In some embodiments, the 3D surface is divided into a plurality of regions, which may have a same size or different sizes. For example, in one embodiment the 3D surface is divided into approximately mm sized regions (e.g., 1 mm square region, 1 mm diameter region, etc.). Alternatively, larger or smaller regions may be used. Surface quality scores may be determined for each region.

At block 1478, processing logic determines that a surface quality score for a region being scanned is below a surface quality threshold. This may include determining one or more surface quality thresholds to apply for the region based on a classification of the region. For example, data associated with a region (e.g., from intraoral scans, the 3D surface, 2D images, etc.) may be input into a trained machine learning model that outputs a dental object classification for the region. Different dental object classifications may be associated with different thresholds. In one embodiment, a rubric is determined based on the dental object classification for the region, where the rubric includes a surface quality threshold to apply. In one embodiment, a region under consideration corresponds to a region of a dental site that is currently in a field of view of the intraoral scanner. In one embodiment, a region under consideration corresponds to a region of the dental site that was recently within the field of view of the intraoral scanner (e.g., was in the field of view less than a threshold amount of time prior to present).

At block 1478, processing logic may additionally or alternatively determine that a threshold amount of time has passed without improvement to the surface quality score or without at least a threshold amount of improvement to the surface quality score.

At block 1480, processing logic may determine surface quality scores for one or more additional regions that are proximate to the region under consideration. In one embodiment, this includes determining surface quality scores for one or more regions that singly or together at least partially surround the region under consideration.

At block 1482, processing logic determines that the surface quality scores for the one or more proximate regions meet or exceed the surface quality score threshold.

At block 1486, based on the determinations made at blocks 1478 and/or 1482, processing logic may determine that additional scans of the region under consideration will not improve the surface quality score for the region.

In some embodiments, at block 1486 processing logic determines a region type for the region. The region type may be a region type associated with failure of the region to meet the surface quality threshold. The region type may be indicative of a reason that the region failed to meet the surface quality threshold. Different region types include, for example: a hole having at least one of a bottom or one or more sidewalls that cannot be imaged by the intraoral scanner; a surface for which an achievable angle of the intraoral scanner relative to the hole is too steep to be imaged by the intraoral scanner; a surface covered by at least one of blood or saliva; and/or a surface covered by a collapsed gum. For some region types, the problem preventing the region from reaching the threshold may be addressable, such as by wiping away blood or saliva, applying dental cord to retract a gum and expose an underlying preparation, and so on. For some region types, the problem preventing the region from reaching the threshold may not be addressable. For example, the region type may be a deep hole, a steep angle, etc. that cannot be addressed. In some embodiments, the region type is determined by application of machine learning or artificial intelligence. For example, one or more intraoral scans, 2D images, a portion of a 3D surface, combination thereof, etc. may be input into a trained machine learning model, which may output a region type for the region.

At block 1488, processing logic may label the region under consideration based on the region type. In some embodiments, processing logic additionally or alternatively labels the region as a void. The void may be a void in terms of lack of data, and may or may not represent an actual physical hole in a 3D surface.

At block 1490, processing logic may output a notice to stop scanning the region and/or to move on to scanning of a next region. Additionally, or alternatively, processing logic may output a notice of the determined region type for the region.

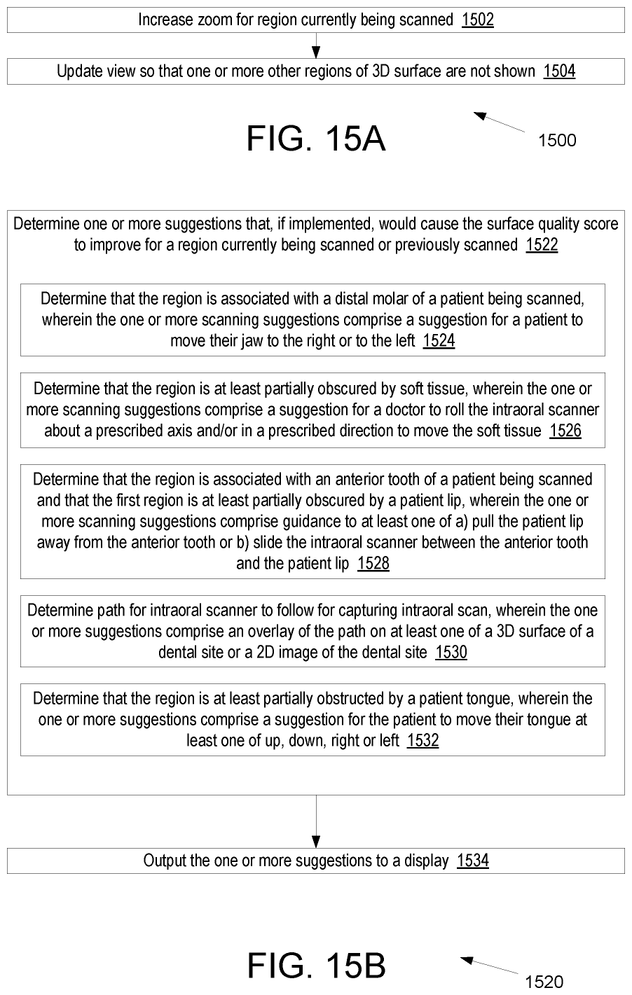

FIG. 15A is a flow chart for a method 1500 of adjusting a zoom setting to assist in intraoral scanning, in accordance with embodiments of the present disclosure. Method 1500 may be performed, for example, at block 1416 of method 1400 and/or at block 1462 of method 1450. At block 1502 of method 1500, processing logic increases a zoom setting for a region currently being scanned. This may include increasing a zoom setting of an entirety of a 3D surface that has been generated thus far. At block 1504, processing logic updates a view of the 3D surface so that the region of the 3D surface is emphasized and shown prominently (e.g., centrally in a display). Due to the increased zoom setting and area of focus, one or more other regions of the 3D surface may not be shown. This is in contrast to a standard technique for showing a 3D surface of a dental site being scanned, which generally includes showing all of the 3D surface.

FIG. 15B is a flow chart for a method 1520 of determining and outputting suggestions to aid in intraoral scanning during the intraoral scanning, in accordance with embodiments of the present disclosure. Method 1520 may be performed, for example, at block 1416 of method 1400 and/or at block 1462 of method 1450. Method 1520 may be performed instead of or in addition to method 1500 in embodiments.

At block 1522 of method 1520, processing logic determines one or more suggestions that, if implemented, would cause the surface quality score to improve for a region currently being scanned or that was previously scanned (e.g., for a region under consideration). In embodiments, a current position of the intraoral scanner in the patient's mouth is determined, and one or more suggestions are determined at least in part on the current position of the intraoral scanner in the patient's mouth. In embodiments, processing logic inputs data for the region of the 3D surface into a trained machine learning model (e.g., a neural network), which may output one or more classification and/or properties for the region. Alternatively, one or more properties of the region may be determined using traditional image processing techniques and/or by applying one or more heuristics to image data and/or surface data. In embodiments, processing logic may determine, for example, whether the region is associated with a distal molar, whether the region is at least partially obscured by soft tissue, whether the region is associated with an anterior tooth, whether the region is at least partially obscured by a patient lip, whether the region is at least partially obscured by a tongue, and so on.

In one embodiment, at block 1524 processing logic determines that the region under consideration (e.g., region currently being scanned) is associated with a distal molar of the patient being scanned. Processing logic may generate one or more scanning suggestions for a patient to move their jaw to the left or to the right. This may include directing the patient to move their jaw to the left and then back to the right, or directing the patient to move their jaw to the right and then back to the left. For example, processing logic may generate an animation of the patient moving their jaw in a particular manner (e.g., to the left or to the right). By having the patient move their jaw in a designated direction, this may cause the patient's jaw to pull a tip of the intraoral scanner into an area being scanned, and may facilitate scanning of the back of the patient's distal molar (e.g., third molar on left or right). Similarly, processing logic may determine that the region under consideration is associated with a distal molar of the patient being scanned, and may generate a suggestion for the doctor to shake the intraoral scanner, which can accomplish a similar outcome as having the patient move their jaw left and/or right.

In one embodiment, at block 1526 processing logic determines that the region under consideration (e.g., region currently being scanned) is obscured by soft tissue. Processing logic may generate one or more scanning suggestions for a doctor to roll the intraoral scanner about a prescribed axis and/or in a prescribed direction to move the soft tissue. This may cause the obscured region of the region to be revealed.

In one embodiment, at block 1528 processing logic determines that the region under consideration (e.g., region currently being scanned) is associated with an anterior tooth of the patient being scanned and is obscured by a patient lip. Processing logic may generate one or more scanning suggestions that include guidance for a doctor to pull the lip of the patient away from the anterior tooth and/or for the doctor to slide the head of the intraoral scanner between the anterior tooth and the patient lip.

In one embodiment, at block 1530 processing logic determines one or more scanner positions/orientations from which intraoral scans should be generated to provide missing details for the region of the 3D surface. Processing logic may then determine a path for the intraoral scanner to follow to capture scans from each of the determined positions/orientations. Processing logic may take into account a current position/orientation of the intraoral scanner relative to the dental site and the distances between the various determined positions/orientations to each other and to the current position/orientation of the intraoral scanner. The determined path may be a most efficient and/or a simplest path to follow from the current position/orientation of the intraoral scanner through each of the determined positions/orientations. The positions/orientations and/or the path may be determined using a trained machine learning model (e.g., a neural network) in embodiments. For example, the current position/orientation of the intraoral scanner and information on one or more regions of the dental site may be input into the trained machine learning model, which may output the determined positions/orientations and/or the determined path. Once the path is determined, an overlay may be generated that shows the path and/or the one or more target positions/orientations for the intraoral scanner that are included in the path. In some embodiments, a generated overlay includes one or more target positions/orientations for the intraoral scanner, but does not include a path for the intraoral scanner to follow. In some embodiments, processing logic generates an animation of the intraoral scanner following the path.

In one embodiment, at block 1532 processing logic determines that the region under consideration (e.g., region currently being scanned) is obscured by a patient tongue. Processing logic may generate one or more suggestions for the patient to move their tongue at least one of up, down, left or right. This may cause the obscured region of the region to be revealed.

At block 1534, processing logic outputs the one or more determined suggestions to the display. In one embodiment, the suggestions are output to the display as text and/or graphics. In one embodiment, the suggestions are output to the display as animations. In one embodiment, the suggestions are output to the display as an overlay that is output over or on top of a 2D image (e.g., viewfinder image) and/or the 3D surface.

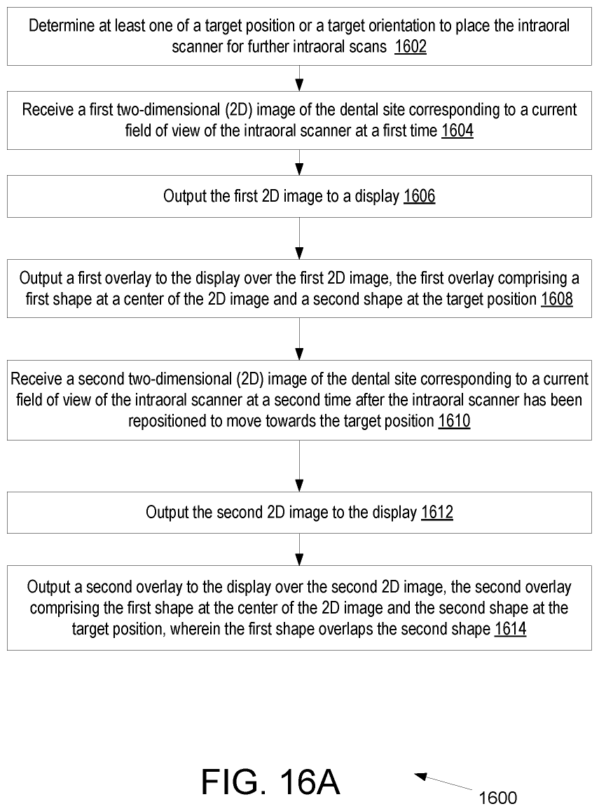

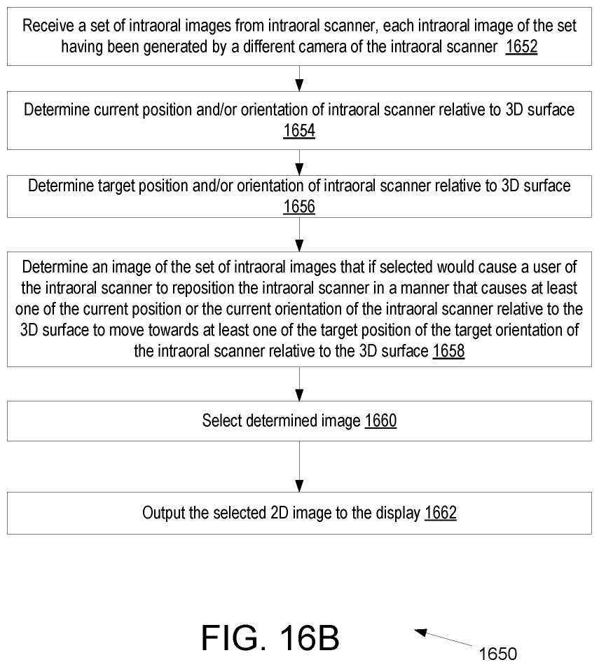

FIG. 16A is a flow chart for a method 1600 of generating and outputting an overlay that provides scan guidance, in accordance with embodiments of the present disclosure. At block 1602 of method 1600, processing logic determines at least one of a target position or a target orientation to place the intraoral scanner for one or more further intraoral scans. The position/orientation of the intraoral scanner may be determined by identifying a region having a low surface quality score (e.g., a surface quality score below a quality threshold), determining where data points are missing for the region, and determining a position/orientation of the intraoral scanner from which the missing data points can be captured.

At block 1604, processing logic receives a first 2D image of the dental site corresponding to a current field of view of the intraoral scanner at a first time. At block 1606, processing logic outputs the first 2D image to a display. At block 1608, processing logic generates a first overlay that includes a first shape approximately at a center of the 2D image or at another position on the 2D image associated with a current field of view of the intraoral scanner (e.g., showing a center of a field of view of the intraoral scanner and/or of a particular camera of the intraoral scanner). The shape may be a cross-hairs, a circle, a ring, a donut, a square, a rectangle, or any other shape. The generated overlay additionally includes a second shape at the target position. The second shape may be a cross-hairs, a circle, a ring, a donut, a square, a rectangle, a rod, a zone, a pyramid, or any other shape. The second shape may be shaped according to the target orientation in embodiments. For example, the second shape may indicate the target orientation and/or an angle between a current orientation and a target orientation. In an example, the first shape may be a ring or hollow circle (e.g., a donut shape), and the second shape may be a solid circle, a cylinder, a pole, etc. The pole may have an orientation associated with the target orientation of the scanner and a position associated with the target position of the scanner. If the angle of the intraoral scanner lines up with the angle of the target orientation, then the cylinder/pole would be shown as a circle. If the angle of the intraoral scanner is at a 90 degree angle to the target orientation, then the cylinder/pole would be shown as roughly a line or rectangle. Once the overlay is generated, it may be output to a display over the first 2D image. Accordingly, the current position/orientation and the target position/orientation for the scanner may be shown in the 2D image.

Responsive to the guidance provided by the overlay, a user may move the intraoral scanner in an attempt to line up the first shape with the second shape. At block 1610, processing logic receives a second 2D image of the dental site corresponding to an updated current field of view of the intraoral scanner at a second time after the intraoral scanner has been repositioned to move towards the target position. At block 1612, processing logic outputs the second 2D image to the display, replacing the first 2D image.

At block 1614, processing logic generates a second overlay that includes the first shape approximately at the center of the 2D image or at the other position on the 2D image associated with the current field of view of the intraoral scanner. The generated second overlay additionally includes the second shape (or a third shape) at the target position. The second/third shape may be shaped according to a difference between a current orientation of the intraoral scanner and the target orientation in embodiments. Since the user moved the scanner closer to the target position, the second shape will be closed to the first shape in the second overlay. Once the second overlay is generated, it may be output to the display over the second 2D image. Accordingly, the new current position/orientation and the target position/orientation for the scanner may be shown in the 2D image.

The operations of blocks 1610-1614 may be repeated as the user moves the intraoral scanner, and with each updated 2D image a new overlay may be generated and output over the updated 2D image. In this manner, the user may be provided guidance with respect to how to move/place the intraoral scanner and how close they are to a target position/orientation. In one embodiment, the scanner is at the target position and orientation when the first shape overlaps the second shape (and optionally shares a common center with and/or is concentric with the second shape). For example, the first shape may be a donut and the second shape may be a circle, and the scanner may reach the target position/orientation when the circle is fully encircled by the donut. In some embodiments, a visualization such as a flash may be output when the scanner reaches the target position/orientation. Other user feedback may additionally or alternatively be provided to indicate that the scanner has reached the target position and orientation, such as using haptic feedback, by outputting a chime or other audio signal, by outputting a visual indicator to the display, and so on.

In some embodiments, in addition to or instead of generating an overlay for output over a 2D image, processing logic generates an overlay for output over the 3D surface. In such an embodiment, a 3D surface may be updated and a view of the updated 3D surface may be output to the display at block 1606 and 1612. The overlay determined at blocks 1608 and 1614 would be similar to those discussed above, but would be projected over the 3D surface in addition to or instead of an overlay being projected over the 2D image. Since the 3D surface contains 3D data as opposed to 2D data, the overlay projected over the 3D surface may contain additional 3D information not included in an overlay projected over the 2D surface, such as depth information. Additionally, the 3D surface includes information for areas outside of the current field of view of the intraoral scanner, so target positions/orientations may be shown on the 3D surface that are outside of the field of view of the scanner and thus that are not viewable in the overlay of the 2D surface.

In some embodiments, an augmented reality system and/or mixed reality system is used, where a doctor may wear an augmented reality display. In such an embodiment, an overlay comprising the first shape and second shape may be projected onto a viewing surface of the augmented reality display (e.g., glasses) directly over the dental site being scanned. This may enable the doctor to continue to focus their attention on the patient while also seeing the current and target positions/orientation of the intraoral scanner relative to the dental site without looking away from the patient. In one embodiment, augmented reality systems are used as set forth in U.S. Pat. No. 10,467,815, issued Nov. 5, 2019, which is incorporated by reference herein in its entirety. In one embodiment, augmented reality systems are used as set forth in U.S. Pat. No. 10,888,399, issued Jan. 12, 2021, which is incorporated by reference herein in its entirety.

FIG. 16B is a flow chart for a method 1650 of selecting an image (e.g., a viewfinder image) to influence how a dental site is scanned, in accordance with embodiments of the present disclosure. As mentioned previously, in some embodiments an intraoral scanner may include multiple cameras (e.g., an array of cameras), each of which may have a different position and/or orientation on the intraoral scanner, and each of which may provide a different point of view of a surface being scanned. Each of the cameras may periodically generate intraoral images (also referred to herein simply as images). A set of images may be generated, where the set may include an image generated by each of the cameras. Processing logic may perform one or more operations on a received set of images to select which of the images to output to a display, and/or which camera to select. The selected image may be the image that will cause a user to position the intraoral scanner in a beneficial manner for capturing of scans of a region being scanned. The selected image may be, for example, a viewfinder image that shows a current field of view a camera of an intraoral scanner.

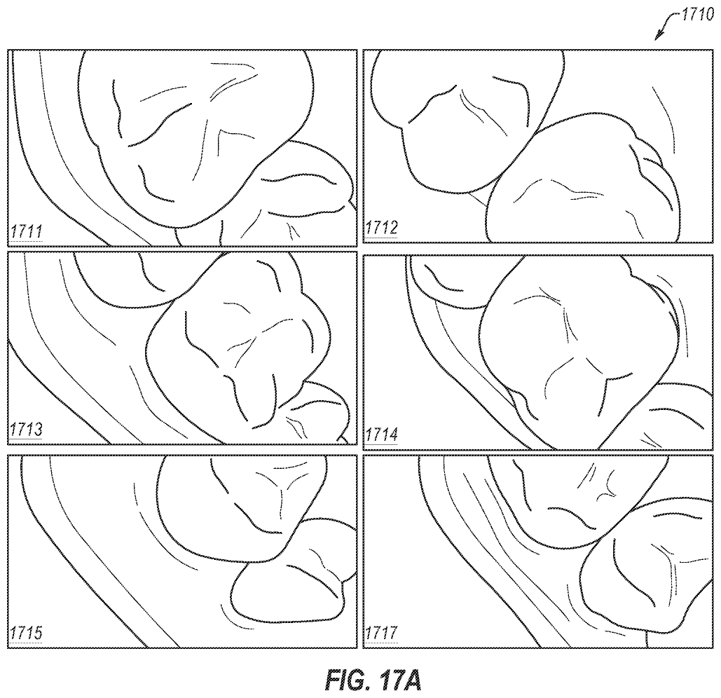

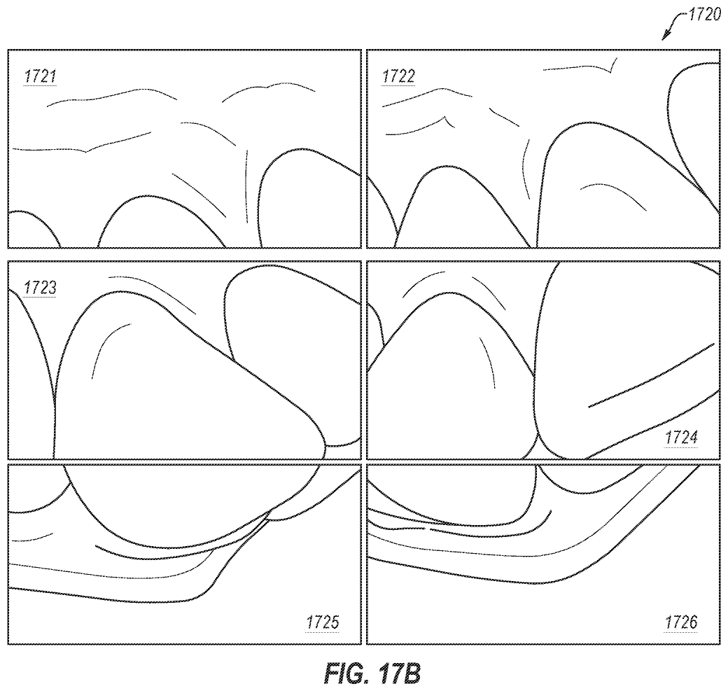

At block 1652 of method 1650, processing logic receives a set of intraoral 2D images from the intraoral scanner. The intraoral 2D images may be color 2D images in embodiments. Alternatively or additionally, the 2D images may be monochrome images, NIR images, or other type of images. Each of the images in the set of images may have been generated by a different camera or cameras at the same time or approximately the same time. For example, the set of images may correspond to images 1711-1717 of FIG. 17A or images 1721-1726 of FIG. 17B.

At block 1654, processing logic may determine a current position and/or orientation of the intraoral scanner relative to the generated 3D surface. At block 1656, processing logic may determine a target position and/or orientation of the intraoral scanner relative to the 3D surface. At block 1658, processing logic may then determine an image of the set of images that if selected would cause a user of the intraoral scanner to reposition the intraoral scanner in a manner that causes the current position and/or orientation to relative to the 3D surface to move towards the target position and/or orientation of the intraoral scanner relative to the 3D surface.

In one embodiment, processing logic determines whether any of the intraoral images of the set of intraoral images satisfies one or more image selection criteria. In one embodiment, the image selection criteria comprise a highest score criterion. Scores (also referred to as values) may be computed for each of the images based on one or more properties of the images, and the image having the highest score may satisfy the image selection criteria. Scores may be determined based on a number of pixels or amount of area in an image having a particular classification in some embodiments. Scores for individual images may be adjusted based on scores of one or more surrounding or other images, such as with use of a weighting matrix in some embodiments. In some embodiments, determining whether any of the intraoral images satisfies one or more criteria includes inputting the set of intraoral images into a trained machine learning model that outputs a recommendation for a selection of a camera associated with one of the input images. Other image selection criteria and/or techniques may also be used. In one embodiment, scores are determined based on where a user is likely to aim the scanner responsive to selection of the image. Each image may be scored based on a distance between where the user is likely to aim the scanner responsive to display of the image and where the user should aim the scanner to reach the target position and/or orientation.

At block 1660, processing logic selects the camera associated with the intraoral image that satisfies the one or more criteria. In one embodiment, the image having a highest score is selected. In one embodiment, an image that was recommended for selection by a machine learning model is selected.

At block 1662, processing logic outputs the intraoral image associated with the selected camera (e.g., the intraoral image having the highest score) to a display. This may provide a user with information on a current field of view of the selected camera, and in turn of the intraoral scanner (or at least a portion thereof). In one embodiment, image selection is determined in accordance with the description of U.S. Patent Application No. 63/434,031, filed Dec. 20, 2022, which is incorporated by reference herein in its entirety. In one embodiment, image selection is determined as set forth in U.S. Patent Application No. 63/434,031, but using different selection rules than those set forth in that patent application.

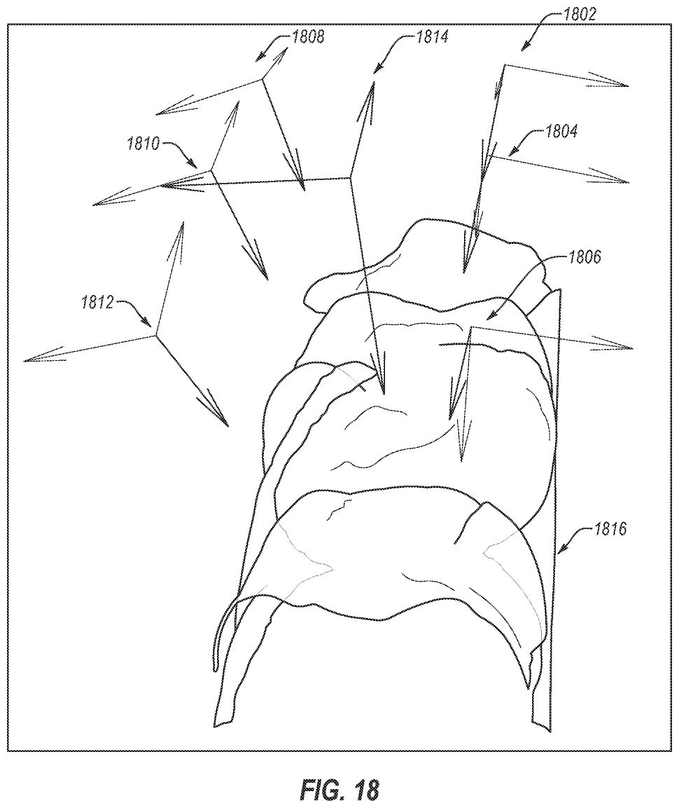

FIG. 17A illustrates 2D images (e.g., intraoral images) 1701, 1702, 1703, 1704, 1705, 1706 of a first dental site generated by an array of cameras of an intraoral scanner, in accordance with embodiments of the present disclosure. In one embodiment, the 2D images are generated, respectively, by cameras having the frames of reference shown in FIG. 18.

FIG. 17B illustrates 2D images 1711, 1712, 1713, 1714, 1715, 1716 of a second dental site generated by an array of cameras of an intraoral scanner, in accordance with embodiments of the present disclosure. In one embodiment, the 2D images are generated, respectively, by cameras having the frames of reference shown in FIG. 18.

FIG. 18 illustrates reference frames of multiple cameras of an intraoral scanner relative to a scanned intraoral object 1816, in accordance with an embodiment of the present disclosure. In the illustrated example, the scanner includes six cameras, each having a distinct frame of reference 1802, 1804, 1806, 1808, 1810, 1812. In some embodiments, a central or average 1814 frame of reference may be computed based on the multiple frames of reference.

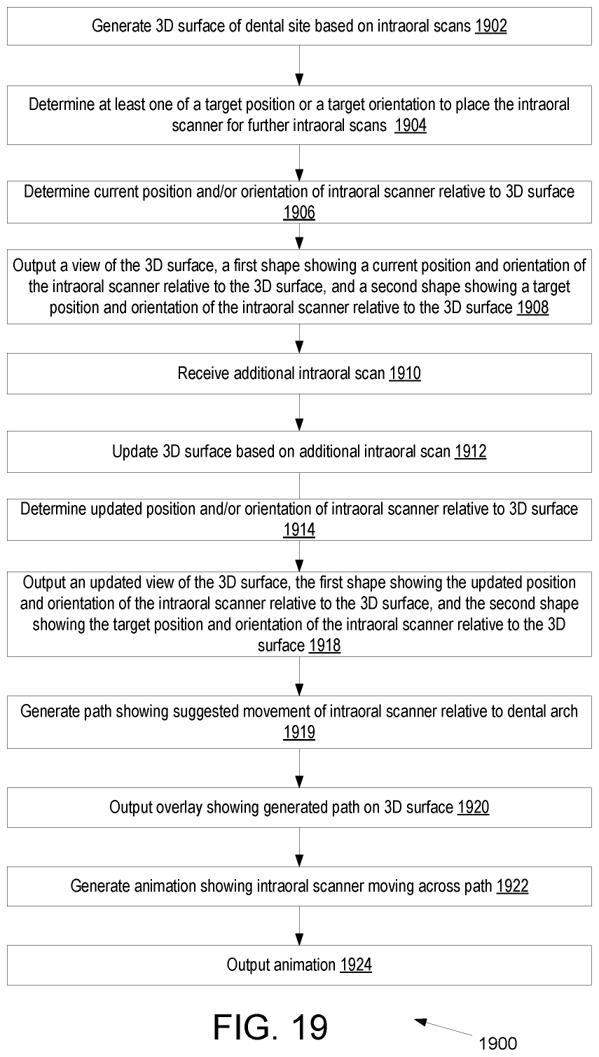

FIG. 19 is a flow chart for a method 1900 of generating and outputting an overlay showing a suggested path to follow during intraoral scanning, in accordance with embodiments of the present disclosure. At block 1902 of method 1900, processing logic generates a 3D surface based on received intraoral scans during intraoral scanning. At block 1904, processing logic determines at least one of a target position or a target orientation to place the intraoral scanner for one or more further intraoral scans. The position/orientation of the intraoral scanner may be determined by identifying a region of the 3D surface having a low surface quality score (e.g., a surface quality score below a quality threshold), determining where data points are missing for the region, and determining a position/orientation of the intraoral scanner from which the missing data points can be captured.

At block 1906, processing logic determines a current position and/or orientation of the intraoral scanner relative to the 3D surface based on one or more most recent intraoral scans. At block 1908, processing logic outputs a view of the 3D surface. Processing logic additionally generates and outputs an overlay comprising a first shape showing a current position and orientation of the intraoral scanner relative to the 3D surface (e.g., such as scanner outline 1350 of FIG. 13A) and a second shape showing the target position and orientation of the intraoral scanner relative to the 3D surface.

The second shape may be shaped according to the target orientation in embodiments. For example, the second shape may indicate the target orientation and/or an angle between a current orientation and a target orientation. In an example, the first shape may be a ring or hollow circle (e.g., a donut shape), and the second shape may be a solid circle, a cylinder, a pole, etc. The pole may have an orientation associated with the target orientation of the scanner and a position associated with the target position of the scanner. If the angle of the intraoral scanner lines up with the angle of the target orientation, then the cylinder/pole would be shown as a circle. If the angle of the intraoral scanner is at a 90 degree angle to the target orientation, then the cylinder/pole would be shown as roughly a line or rectangle. Once the overlay is generated, it may be output to a display over the first 2D image. Accordingly, the current position/orientation and the target position/orientation for the scanner may be shown in the 2D image.

Responsive to the guidance provided by the overlay, a user may move the intraoral scanner in an attempt to line up the first shape with the second shape. At block 1910, processing logic receives one or more additional intraoral scans of the dental site after the intraoral scanner has been repositioned to move towards the target position. At block 1912, processing logic updates the 3D surface based on the additional intraoral scan(s).

At block 1914, processing logic determines an updated position and/or orientation of the intraoral scanner relative to the 3D surface. At block 1918, processing logic generates an updated overlay comprising the first shape (or a third shape) showing a current position and orientation of the intraoral scanner relative to the 3D surface and the second shape (or a fourth shape) showing the target position and orientation of the intraoral scanner relative to the 3D surface. Processing outputs an updated view of the 3D surface with the updated overlay laid over the view of the 3D surface. The second/fourth shape may be shaped according to a difference between a current orientation of the intraoral scanner and the target orientation in embodiments. Since the user moved the scanner closer to the target position, the second shape will be closer to the first shape in the second overlay.

The operations of blocks 1910-1918 may be repeated as the user moves the intraoral scanner, and with each updated 3D surface and/or new intraoral scan a new overlay may be generated and output over the updated 3D surface. In this manner, the user may be provided guidance with respect to how to move/place the intraoral scanner and how close they are to a target position/orientation. In one embodiment, the scanner is at the target position and orientation when the first shape overlaps the second shape (and optionally shares a common center with and/or is concentric with the second shape). For example, the first shape may be a donut and the second shape may be a circle, and the scanner may reach the target position/orientation when the circle is fully encircled by the donut. In some embodiments, a visualization such as a flash may be output when the scanner reaches the target position/orientation. Other user feedback may additionally or alternatively be provided to indicate that the scanner has reached the target position and orientation, such as using haptic feedback, by outputting a chime or other audio signal, by outputting a visual indicator to the display, and so on.

In one embodiment, at block 1919 a path showing a suggested movement of the intraoral scanner relative to the dental arch to cause the intraoral scanner to move from the current position/orientation to the target position/orientation is generated. At block 1920, an overlay showing the generated path may be output over the 3D surface. In one embodiment, at block 1922 an animation showing the intraoral scanner moving along the path is generated. The animation may then be shown at block 1924.

The path, animation and/or target shape may indicate to a user of the intraoral scanner exactly how to move/reposition the scanner to capture a region of a dental site that the user is having difficulty capturing. For example, the path, animation and/or target shape placement may indicate to a user how to rotate the scanner, when to rotate the scanner, and so on. In some embodiments, processing logic initially provides gross instructions as to how to position/move the scanner, and as the user gets closer to the target position/orientation more detailed instructions (e.g., a zoomed in view of the region of the 3D surface and the target shape of the overlay) are output.

In some embodiments, there may be multiple regions that need additional scanning (e.g., have low surface quality scores). In embodiments, target positions/orientations of the scanner may be determined for each of these regions. Processing logic may then determine the path taking into account the multiple target positions/orientations. By following the path, a user may generate the needed scans for each of these regions.

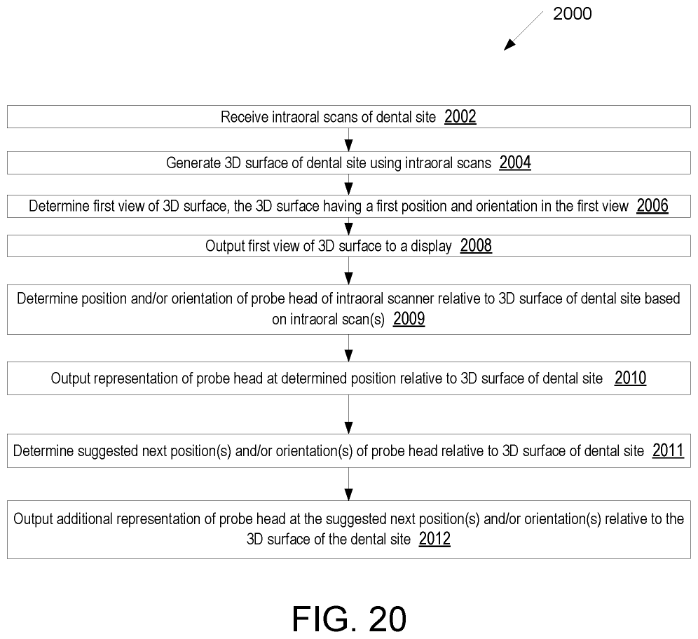

FIG. 20 is a flow chart for a method 2000 of guiding a user of an intraoral scanner as to where to position the intraoral scanner during scanning, in accordance with embodiments of the present disclosure. At block 2002, processing logic receives intraoral scans of a dental site (e.g., of an upper or lower jaw). At block 2004, processing logic generates a 3D surface of the dental site using the intraoral scans. At block 2006, processing logic determines a first view of the 3D surface, the 3D surface having a first position and orientation in the first view. At block 2008, processing logic outputs the first view of the 3D surface to a display.

At block 2009, processing logic determines a position and/or orientation of a probe head of the intraoral scanner relative to the 3D surface of the dental site based on the intraoral scans (e.g., based on a most recent intraoral scan that has been successfully registered and stitched to the 3D surface). At block 2010, processing logic may output, to the display, a representation of the probe head at the determined position and/or orientation relative to the 3D surface.

At block 2011, processing logic determines a suggested next position and/or orientation of the probe head relative to the 3D surface of the dental site. In one embodiment, the suggested next position and orientation of the probe may be determined based on a difficulty to scan a particular upcoming region of the dental site that is yet to be scanned. In some embodiments, one or more previously generated 3D models of the dental site (e.g., generated during previous patient visits) may be accessible to processing logic. Processing logic may assess these one or more 3D models to determine tooth crowding and/or a tooth geometry that is particularly challenging to scan. Accordingly, processing logic may determine a suggested scanning speed, a suggested position and/or orientation of the scanner to capture difficult to scan regions, a sequence of suggested positions and/or orientations of the scanner to capture the difficult to scan regions, and so on. In one embodiment, processing logic determines a suggested trajectory for the intraoral scanner, which may include a sequence of recommended positions and orientations of the intraoral scanner.

At block 2012, processing logic outputs an additional representation of the probe head at the suggested position(s) and/or orientation(s) relative to the 3D surface of the dental site. The representation of the probe head that shows a current position and orientation of the probe head may be shown using a first visualization and the additional representation of the probe head that shows a suggested next position and orientation of the probe head may be shown using a second visualization that is different from the first visualization. The first visualization may include a first color, a first transparency level, a first line type, a first zoom level (also referred to as magnification level), etc., and the second visualization may include a second color, a second transparency level, a second line type, a second zoom level (also referred to as magnification level), etc. In one embodiment, processing logic shows the additional representation of the probe head moving according to the determined recommended trajectory for the intraoral scanner. In embodiments, suggestions for a trajectory or path for the intraoral scanner to follow in scanning a region of a dental site (or a sequence of regions of the dental site) may be determined and displayed as set forth in U.S. application Ser. No. 17/894,096, filed Aug. 23, 2022, which is incorporated by reference herein in its entirety.

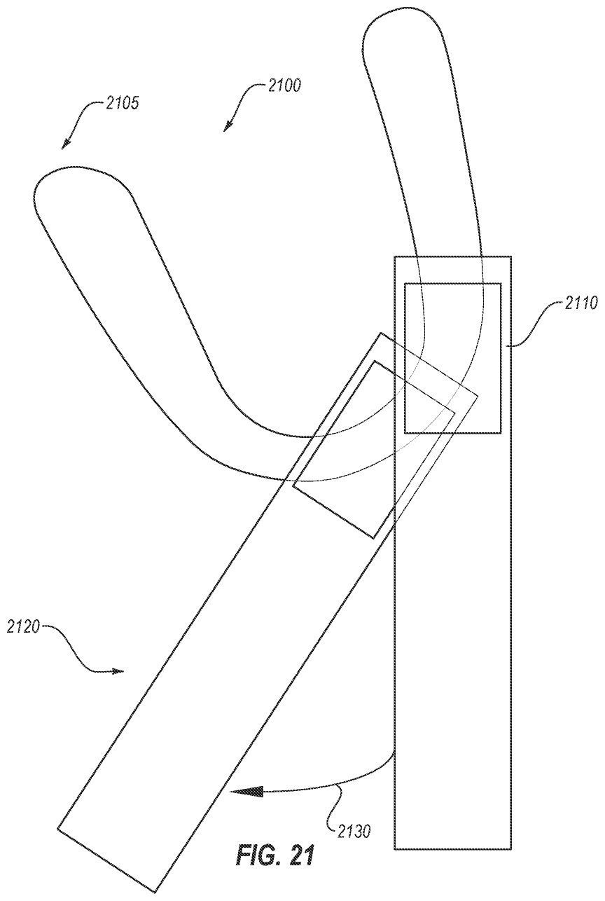

FIG. 21 illustrates a view of a 3D surface of a dental site being scanned, a position and orientation of a probe head of an intraoral scanner relative to the 3D surface, and a suggested next position and orientation of the probe head relative to the 3D surface, in accordance with an embodiment of the present disclosure. As shown, a first visualization may be used for the current position and orientation of the probe head 2110 and a second visualization may be used for the recommended position and orientation of the probe head 2120. In embodiments, an animation of the probe head 2120 moving through a sequence of positions and orientations according to a recommended trajectory 2130 is shown.

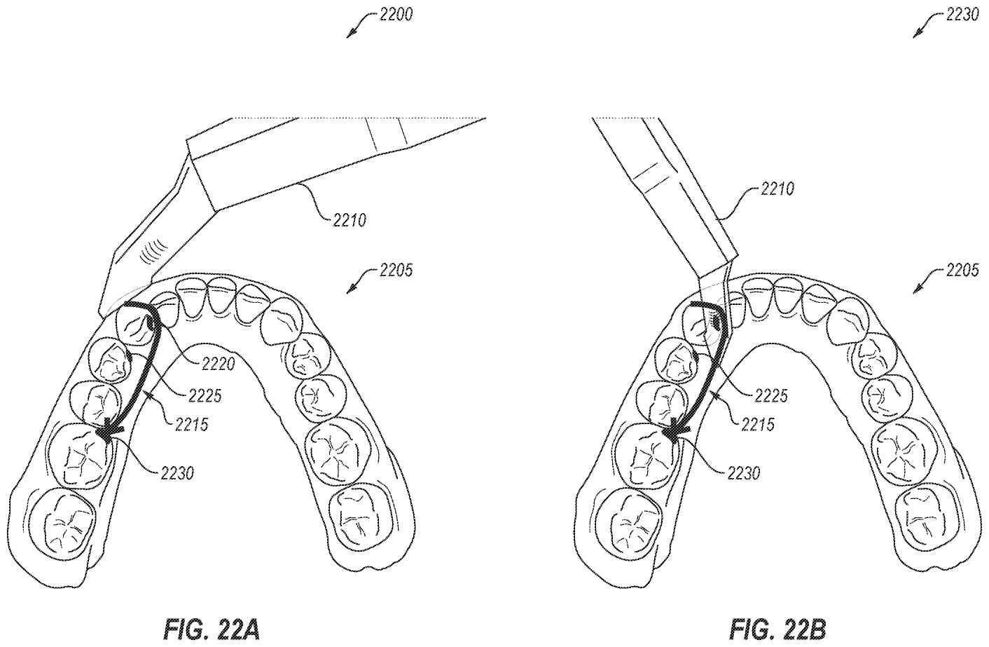

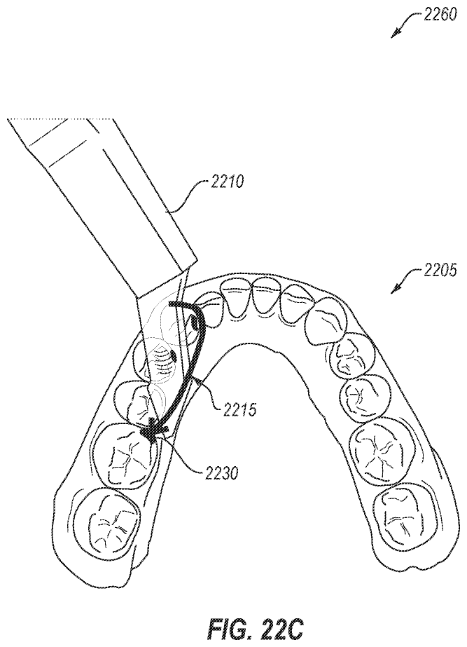

FIGS. 22A-C illustrate a view of a 3D surface 2205 of a dental site being scanned and a suggested path 2215 for scanning the dental site, in accordance with an embodiment of the present disclosure. As shown, multiple regions 2220, 2225, 2230 have a low surface quality and/or have voids. Accordingly, additional scanning should be performed for these regions 2220-2230. FIG. 22A illustrates a view of 3D surface 2205 and an initial position and orientation of a probe head of intraoral scanner 2210 relative to the 3D surface 2205. The suggested path moves first to a first target position/orientation of the intraoral scanner 2210 that will capture region 2220, then to a second target position/orientation of the intraoral scanner 2210 that will capture region 2225, and finally to a third target position/orientation of the intraoral scanner 2210 that will capture region 2230. The path begin with the region 2220 closest to intraoral scanner 2210 and moves across a most efficient path to capture all of the regions having low surface quality scores and/or voids in embodiments. FIG. 22B illustrates a view of 3D surface 2205 after scanner 2210 has moved past the target position/orientation suggested for scanning region 2220 and to the target position/orientation suggested for scanning region 2225. FIG. 22C illustrates a view of 3D surface 2205 after scanner 2210 has moved to the target position/orientation suggested for scanning region 2230. In embodiments, an animation may be presented showing of the scanner 2210 moving through the sequence of positions and orientations shown in FIGS. 22A-22C.

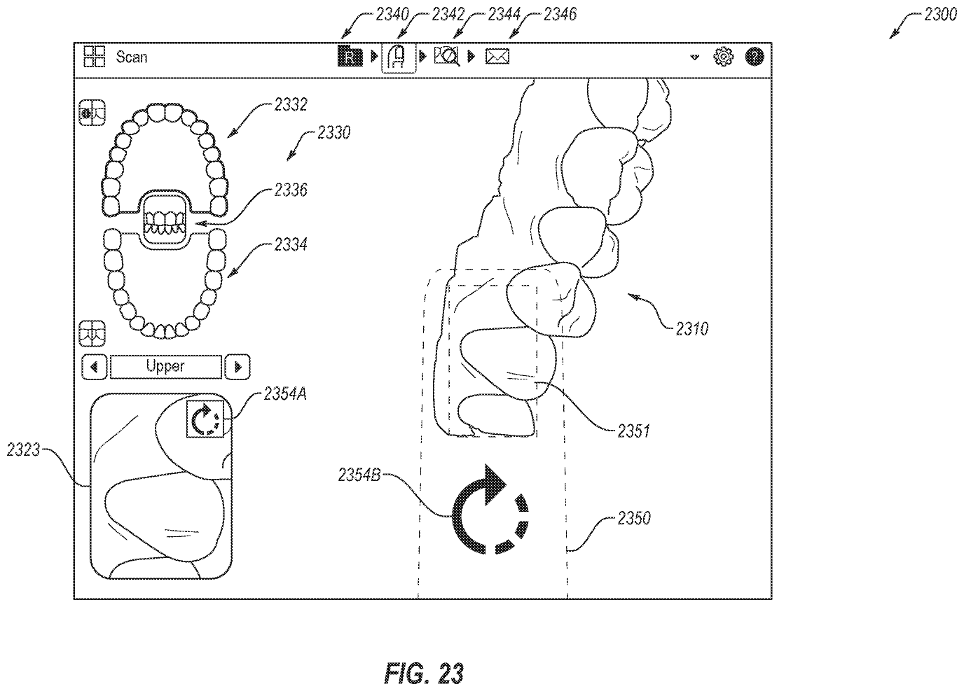

FIG. 23 illustrates a view of a 3D surface 2310 of a dental site being scanned and a suggestion to rotate an intraoral scanner, in accordance with an embodiment of the present disclosure. FIG. 23 illustrates a view of a graphical user interface 2300 for an intraoral scan application that includes 3D surface 2310 and a 2D image 2323 of a current field of view (FOV) of an intraoral scanner, in accordance with embodiments of the present disclosure. Additionally, an outline 2350 of the intraoral scanner, including an outline 2351 of a field of view of the intraoral scanner may be shown relative to the 3D surface 2310. The 3D surface 2310 is generated by registering and stitching together multiple intraoral scans captured during an intraoral scanning session. As each new intraoral scan is generated, that scan is registered to the 3D surface and then stitched to the 3D surface. Accordingly, the 3D surface becomes more and more accurate with each intraoral scan, until the 3D surface is complete. A 3D model may then be generated based on the intraoral scans.

Some regions of a dental arch may be particularly challenging to scan. For example, regions with deep pockets or valleys and distal most molars may be particularly challenging to scan. In some embodiments, processing logic may analyze 3D surface 2310 or data from 3D surface (e.g., surface quality score data) and determine that a user is having difficulty scanning a region. Other clues such as a user pausing at a region, a region not improving in surface quality over time, etc. may also indicate that a user is having difficulty scanning a region. Processing logic may determine one or more suggestions that, if implemented, will likely result in an increased surface quality score for a region being scanned. One such suggestion may be to rotate the intraoral scanner about a particular axis and/or in a particular direction. As illustrated, a suggestion to rotate the intraoral scanner may be shown as an overlay on 2D image 2323 and/or as a graphic on the depiction of intraoral scanner 2350. The suggestion may be presented as a rotate icon 2354A and/or rotate icon 2354B. The rotate icon 2354A-B may indicate the direction of rotation, the axis of rotation and/or an amount of rotation in embodiments.

In one embodiment, as shown, a scan segment indicator 2330 may include an upper dental arch segment indicator 2332, a lower dental arch segment indicator 2334 and a bite segment indicator 2336. The GUI of the intraoral scan application may further include a task bar with multiple modes of operation or phases of intraoral scanning. Selection of a patient selection mode 2340 may enable a doctor to input patient information and/or select a patient already entered into the system. Selection of a scanning mode 2342 enables intraoral scanning of the patient's oral cavity. After scanning is complete, selection of a post processing mode 2344 may prompt the intraoral scan application to generate one or more 3D models based on intraoral scans and/or 2D images generated during intraoral scanning, and to optionally perform an analysis of the 3D model(s). Selection of an prescription fulfillment mode 2346 may cause the generated orthodontic and/or prosthodontic prescriptions to be sent to a lab or other facility to cause a prosthodontic device (e.g., a crown, bridge, denture, etc.) or orthodontic device (e.g., an orthodontic aligner) to be generated.

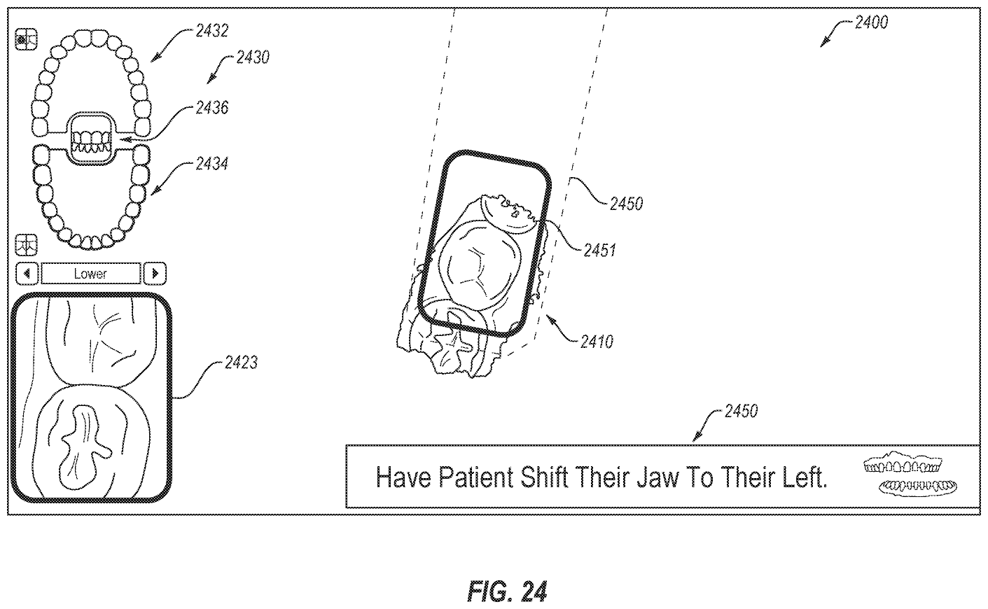

FIG. 24 illustrates a view of a 3D surface 2410 of a dental site being scanned and a suggestion for a patient to move their jaw, in accordance with an embodiment of the present disclosure. FIG. 24 illustrates a view of a graphical user interface 2400 for an intraoral scan application that includes 3D surface 2410 and a 2D image 2423 of a current field of view (FOV) of an intraoral scanner, in accordance with embodiments of the present disclosure. Additionally, an outline 2450 of the intraoral scanner, including an outline 2451 of a field of view of the intraoral scanner may be shown relative to the 3D surface 2410.

Some regions of a dental arch may be particularly challenging to scan. For example, regions with deep pockets or valleys and distal most molars may be particularly challenging to scan. In some embodiments, processing logic may analyze 3D surface 2410 or data from 3D surface (e.g., surface quality score data) and determine that a user is having difficulty scanning a region. Other clues such as a user pausing at a region, a region not improving in surface quality over time, etc. may also indicate that a user is having difficulty scanning a region. Processing logic may determine one or more suggestions that, if implemented, will likely result in an increased surface quality score for a region being scanned. One such suggestion may be for a patient to move their jaw left and/or right (e.g., left then right, or right then left). As illustrated, a suggestion for the patient to move their jaw may be shown in a pop-up window 2450, and may include text and/or graphics.

In one embodiment, as shown, a scan segment indicator 2430 may include an upper dental arch segment indicator 2432, a lower dental arch segment indicator 2434 and a bite segment indicator 2436.

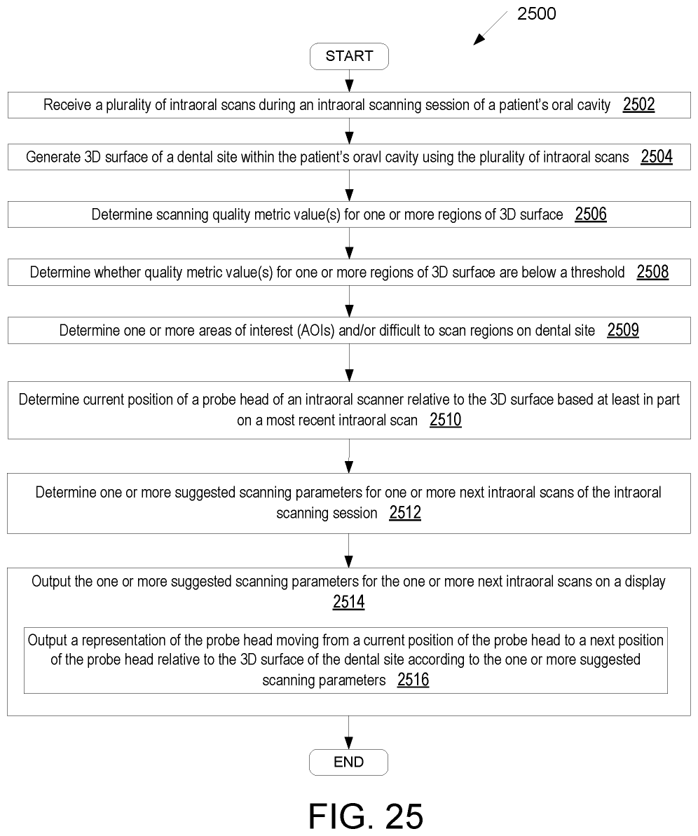

FIG. 25 is a flow chart illustrating an embodiment for a method 2500 of determining scanning quality for regions of a 3D surface during intraoral scanning and outputting suggestions for improving the scanning quality. At block 2502 of method 2500, processing logic receives a plurality of intraoral scans during an intraoral scanning session of a patient's oral cavity. At block 2504, processing logic generates a 3D surface of a dental site within the patient's oral cavity using the plurality of intraoral scans. At block 2506, processing logic determines scanning quality metric values (e.g., surface quality scores) for one or more regions of the 3D surface and/or for one or more received intraoral scans. At block 2508, processing logic determines whether the scanning quality metric values (e.g., surface quality scores) for one or more regions of the 3D surface and/or for one or more intraoral scans is below a scanning quality threshold (or otherwise fail to satisfy one or more scan quality criteria). At block 2509, processing logic may additionally identify one or more areas of interest of the dental site that is yet to be scanned or that has only partially been scanned. Processing logic may additionally or alternatively determine one or more difficult to scan regions that has yet to be scanned or that is only partially scanned. In one embodiment, areas of interest (AOIs) and/or difficult to scan regions are determined based on analysis of a previously generated 3D model of the dental site (e.g., of the patient's dental arch). For example, a doctor may have scanned the patient's dental arches during prior patient visits or earlier in the current patient visit. Processing logic may analyze such a 3D model to identify tooth crowding, areas with little clearance (e.g., the buccal side of the back molars), and so on.

At block 2510, processing logic determines a current position (and optionally one or more past positions) of a probe head of an intraoral scanner relative to the 3D surface based at least in part on a most recent intraoral scan that successfully stitched to the 3D surface. At block 2512, processing logic determines one or more suggested scanning parameters for one or more next intraoral scans of the intraoral scanning session. The scanning parameters may include a relative position and/or orientation of the intraoral scanner probe head relative to a portion of the dental site to be scanned next. Scanning parameters may additionally include a speed with which to move the intraoral scanner, a distance of the scanner from the dental site, an angle of the scanner relative to the dental site, and so on. Processing logic may additionally determine one or more unscanned regions of the patient's oral cavity. Additionally, processing logic may determine scanning quality metric values for already scanned regions, and may identify those regions with one or more scanning quality metric values that are outside of target ranges for the scanning quality metric values. Additionally, processing logic may identify one or more AOIs on the 3D surface.

At block 2514, processing logic outputs the one or more suggested scanning parameters for the one or more next intraoral scans on a display (e.g., in a GUI of an intraoral scan application). The one or more suggested scanning parameters may include, for example, a suggested next position and/or orientation of the probe head of the intraoral scanner relative to the 3D surface, a next distance between the probe head and the 3D surface, a speed of movement between a current position of the probe head and a next position of the probe head, and so on. The suggested next position(s) and/or orientation(s) of the probe head relative to the patient's oral cavity (e.g., dental arch) and/or other suggested scanning parameters may be positions and/or orientations suitable to scan the one or more unscanned regions, to rescan AOIs, and so on. For example, suggested next positions and/or orientations of the probe head relative to the patient's oral cavity may be positions and/or orientations and/or other suggested scanning parameters suitable to rescan already scanned regions with scanning quality metric values that failed to satisfy a scanning quality criterion (e.g., that were outside of target scanning quality metric value ranges). The suggested scanning parameters (e.g., position and/or orientation of the scan head, scan speed, scan distance, scan angle, etc.), when used, may cause one or more of the scan quality metric values to increase for the regions having the unacceptable scan quality metric values. Additionally, or alternatively, the suggested next position(s) and/or orientation(s) of the probe head relative to the patient's oral cavity (e.g., dental arch) and/or other suggested scanning parameters may be positions and/or orientations suitable to re-scan the AOIs. In one embodiment, at block 2516 processing logic outputs a representation of the probe head moving from a current position of the probe head to a next position of the probe head relative to the 3D surface of the dental site according to the one or more suggested scanning parameters.

In one example, processing logic determines a current angle of the probe head relative to the 3D surface, and may determine whether the angle of the probe head is within a target angle range (e.g., 40-60 degrees) relative to the probe head. Responsive to determining that the angle of the probe head is outside of the target angle range, processing logic may determine one or more angle adjustments for the probe head, where the one or more suggested scanning parameters may include the one or more angle adjustments.

In one example, processing logic determines a ratio of distal surfaces to mesial surfaces represented in the 3D surface of the dental site. Based on the ratio of the distal surfaces to the mesial surfaces, processing logic may determine whether the distal surfaces or the mesial surfaces are dominant. Responsive to determining that the distal surfaces are dominant, processing logic may determine one or more first angle adjustments for the probe head that will increase an amount of captured mesial surfaces. Responsive to determining that the mesial surfaces are dominant, processing logic may determine one or more second angle adjustments for the probe head that will increase an amount of captured distal surfaces. Processing logic may then determine one or more suggested scanning parameters that comprise the one or more first angle adjustments or the one or more second angle adjustments.

In one embodiment, processing logic determines a scanning speed associated with one or more intraoral scans and/or one or more regions of the 3D surface. Processing logic may determine that the scanning speed is outside of a scanning speed range, and may suggest one or more scanning parameters for the one or more next intraoral scans that will cause the scanning speed to fall within the target scanning speed range.

In one embodiment, processing logic determines a trajectory of the intraoral scanner during intraoral scanning. Processing logic projects the trajectory into the future, and optionally compares the areas to be scanned to a 3D model of the dental site that was previously generated. Processing logic may determine whether an upcoming area to be scanned is a difficult to scan region or an easy to scan region. If a difficult to scan region is upcoming in the intraoral scanning session, then processing logic may output an alert for a user to slow down a scan speed (e.g., to slow down a speed of the probe head) for scanning of the difficult to scan region. Processing logic may additionally determine one or more suggested scanning parameters for scanning the difficult to scan region (other than scan speed), and may output suggestions to use the one or more suggested scanning parameters.

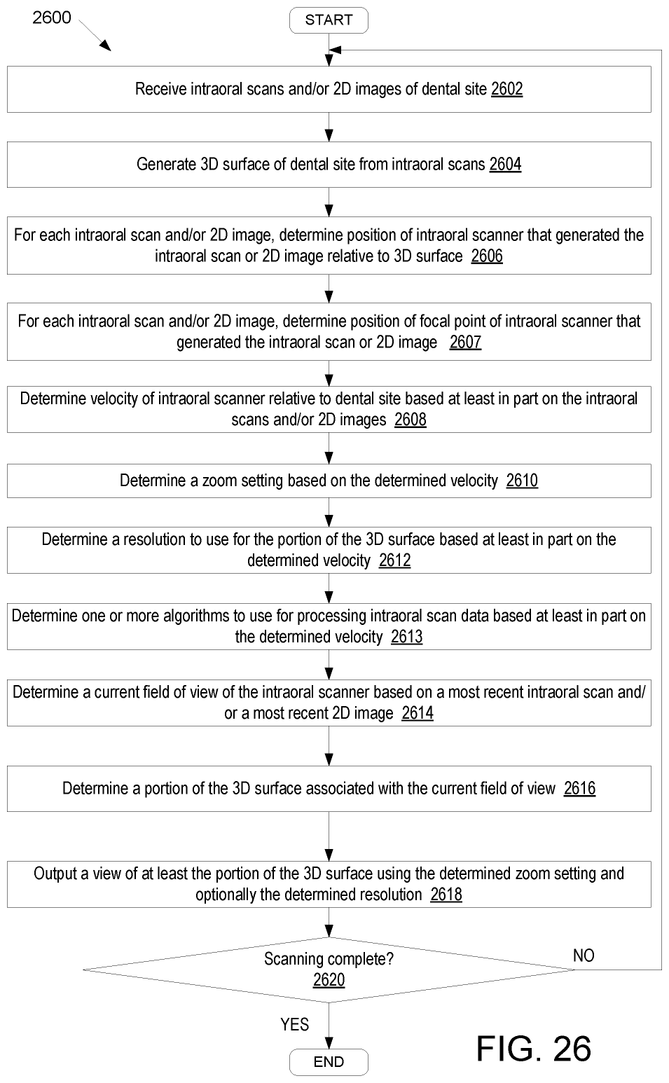

FIG. 26 is a flow chart for a method 2600 of adjusting a zoom setting of a 3D surface based on scanner velocity, in accordance with embodiments of the present disclosure. As a user moves an intraoral scanner during intraoral scanning, the amount of information that the user might find useful for a particular region (e.g., a region currently being scanned) may be correlated to a velocity at which the user moves the intraoral scanner. For example, if the user slows down the movement of the intraoral scanner (e.g., pauses the intraoral scanner to capture a preparation tooth or a difficult to capture tooth), it may be beneficial for the user to have more information about the region (e.g., tooth) that the scanner is currently focused on. Accordingly, in embodiments a zoom setting for a generated 3D surface is automatically controlled (e.g., controlled without user input) based at least in part on a velocity of the intraoral scanner.

At block 2602 of method 2600, receives one or more intraoral scans and/or associated 2D images of a dental site. At block 2604, processing logic generates a 3D surface of the dental site from the intraoral scans. Optionally the 2D images may also be used in the generation of the 3D surface (e.g., to provide color information for the 3D surface).

At block 2606, for each intraoral scan and/or 2D image, processing logic determines a position of the intraoral scanner that generated the intraoral scan or 2D image relative to the 3D surface. Since intraoral scans include many points with distance information indicating distance of those points in the intraoral scan to the intraoral scanner, the distance between the intraoral scanner to the dental site (and thus to the 3D surface to which the intraoral scans are registered and stitched) is known and/or can be easily computed. The intraoral scanner may alternate between generating intraoral scans and 2D images, and so the distance between the intraoral scanner and the dental site (and/or the 3D surface) that is associated with a 2D image may be interpolated based on distances associated with intraoral scans generated before and after the 2D image in embodiments.

At block 2607, for each intraoral scan and/or 2D image, processing logic may determine a position of a focal point of the intraoral scanner and/or virtual point at a set distance from the intraoral scanner that generated the intraoral scan/2D image. In one embodiment, the focal point and/or virtual point is a point about 10 mm from the intraoral scanner at an x,y position that is approximately at a center of a field of view of the intraoral scanner.

At block 2608, processing logic determines a velocity of the intraoral scanner relative to the dental site based at least in part on the intraoral scans and/or 2D images. In one embodiment, processing logic determines a difference between positions of the intraoral scanner associated with multiple intraoral scans/2D images, and determines the velocity based on the difference between the determined positions and a difference in time between when the intraoral scans/2D images were generated. In one embodiment, processing logic determines a difference between positions of the focal point and/or virtual point of the intraoral scanner associated with multiple intraoral scans/2D images, and determines the velocity based on the difference between the determined positions and a difference in time between when the intraoral scans/2D images were generated.

At block 2610, processing logic determines a zoom setting based on the determined velocity (e.g., of the intraoral scanner and/or of the focal point of the intraoral scanner). In one embodiment, the determined velocity is used as a key to perform a lookup in a lookup table. The lookup table may associate velocities with zoom settings. Accordingly, processing logic may determine an entry in the lookup table associated with the determined velocity, and may determine the zoom setting in that entry. In one embodiment, the zoom setting is determined by inputting the velocity into a function that relates velocity to zoom setting. In embodiments, the zoom setting is inversely proportional to the velocity. Accordingly, as the intraoral scanner speeds up, processing logic zooms out of the 3D surface. Similarly, as the intraoral scanner slows down and/or pauses, processing logic zooms in on the 3D surface.

In one embodiment, at block 2612 processing logic determines a resolution to use for a portion of the 3D surface and/or to use for an intraoral scan based on the determined velocity (e.g., of the intraoral scanner and/or of the focal point of the intraoral scanner). In one embodiment, the determined velocity is used as a key to perform a lookup in a lookup table. The lookup table may associate velocities with resolution settings. Accordingly, processing logic may determine an entry in the lookup table associated with the determined velocity, and may determine the resolution setting in that entry. In one embodiment, the resolution setting is determined by inputting the velocity into a function that relates velocity to resolution.

In one embodiment, at block 2613 processing logic determines one or more algorithms to use for processing intraoral scan data (e.g., intraoral scans, 2D images, regions of 3D surfaces, etc.). Examples of algorithms include registration algorithms, stitching algorithms, moving tissue detection and removal algorithms, object detection algorithms, soft tissue detection algorithms, and so on.

At block 2614, processing logic determines a current field of view of the intraoral scanner. The determined field of view may be a combined field of view of multiple cameras of the intraoral scanner as determined from a most recently received set of 2D images. Alternatively, the field of view may be the field of view associated with a most recently received intraoral scan.

At block 2616, processing logic determines a portion of the 3D surface associated with the current field of view. This may be the portion or region of the 3D surface that the intraoral scanner is currently focused on (e.g., the region currently being imaged).

At block 2618, processing logic outputs a view of at least the determined portion/region of the 3D surface using the determined zoom setting, and optionally using a determined resolution. The view may be, for example, an occlusal view, a birds-eye view, a distal-to-mesial view, a mesial-to-distal view, and so on. Processing logic may also process the region of the 3D surface and/or intraoral scan data used to generate the region of the 3D surface using the one or more selected algorithms. Determining on the current zoom setting, some of the 3D surface may not be displayed. For example, if processing logic zooms in on a particular region, then that region may be shown along with surrounding areas that are proximate to the region. However, other regions that are further away from the region presently being scanned may not be visible. In some embodiments, processing logic determines whether the velocity is below a velocity threshold. If the velocity is below the velocity threshold, processing logic may display a second view of the 3D surface together with a first view of the 3D surface. The two different views of the 3D surface may have different zoom settings and/or different pan/rotation settings. Accordingly, the first view of the 3D surface may be a zoomed in view of the 3D surface from a first angle (e.g., corresponding to a current angle of the intraoral scanner relative to the dental site) and the second view of the 3D surface may be a zoomed in view of the 3D surface from a different angle (e.g., an occlusal view, lingual view, etc.). In some embodiments, additional information is output to the display responsive to the velocity falling below a threshold velocity. Such information may include an icon and/or window that indicates surface quality scores, an indication of regions with missing data, and so on.

At block 2620, processing logic determines whether scanning is complete. If scanning is not complete, the method returns to block 2602, and operations 2602-2618 are repeated. This process may repeat as long as scanning is ongoing. Accordingly, processing logic may automatically zoom in and out as scanning is being performed as the velocity of the intraoral scanner changes. If the user slows down movement of the scanner, processing logic may zoom in on a current region to show that region in greater details, and if the user speeds up movement of the scanner, processing logic may zoom out to provide a view of more of the 3D surface.

In some embodiments, a user may enable or disable the automatic zoom function via the GUI for the intraoral scan application. In one embodiment, processing logic may provide a suggestion to activate the automatic zoom functionality if processing logic detects that a user has manually zoomed in and/or out one or more times during intraoral scanning. In one embodiment, processing logic outputs a suggestion to activate the automatic zoom functionality responsive to detecting that a user is having trouble scanning a region of a dental site.

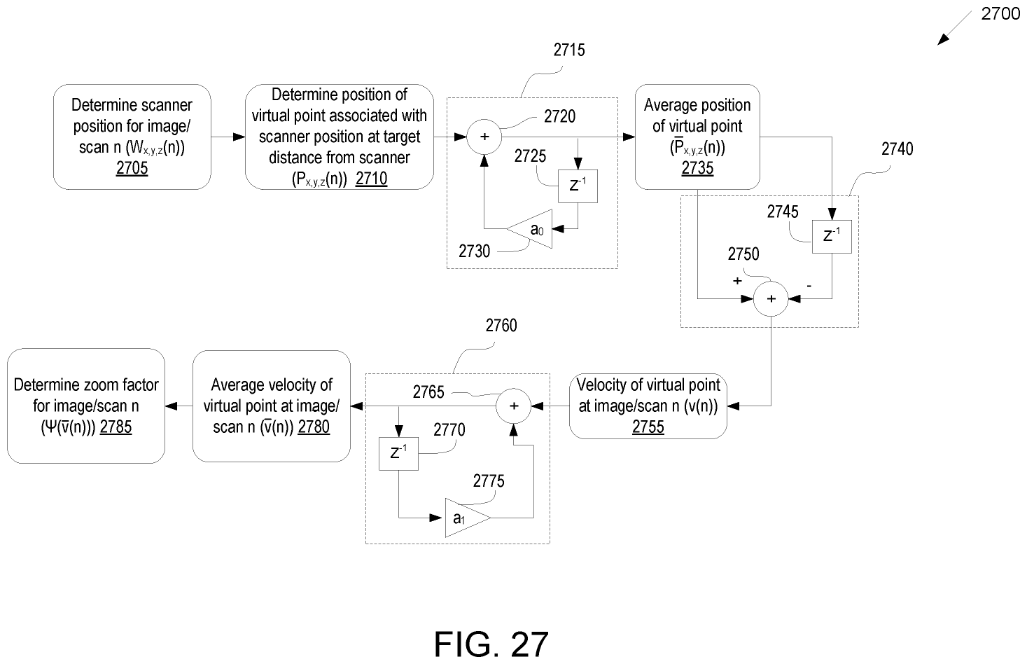

FIG. 27 is a flow chart for a method 2700 of adjusting a zoom setting of a 3D surface based on scanner velocity, in accordance with embodiments of the present disclosure. At block 2705 of method 2700, processing logic determines a scanner position for an image and/or intraoral scan received from an intraoral scanner during intraoral scanning. The scanner position may be a 3D position, and may be represented as W_{x,y,z}(n), where W is the scanner position and n is an image or intraoral scan. In embodiments, the scanner position may be continuously or periodically estimated, allowing concatenations of individual frames, for example using a process such as chain-stitching. At block 2710, processing logic determines a position of a virtual point associated with the scanner position, where the virtual point is at a target distance from the intraoral scanner. For example, the position of the virtual point may be associated with a focal point of the intraoral scanner. The virtual point provides a reliable estimation of how the field of view of the intraoral scanner changes over time. The virtual point may be a point at approximately the center of the FOV of the intraoral scanner at a typical depth. In one embodiment, the virtual point is a point about 10 mm from the intraoral scanner within the field of view of the intraoral scanner. Accordingly, the virtual point may have a position of x,y,z={0,0,10} in a scanner coordinate system. The virtual point position may be represented as P_{x,y,z}(n)=F(W_{x,y,z}(n)), where P is the position of the virtual point in the world coordinate system (e.g., coordinate system of the 3D surface) and F is a function applied to the scanner position to determine the position of the virtual point in the world coordinate system.

In embodiments, the virtual position of the point may frequently change as scanning progresses (e.g., as each new intraoral scan and/or 2D image is generated). This may cause the location of the virtual position to be jerky. Accordingly, in embodiments a smoothing operation is performed to smooth the location of the virtual point over time. In one embodiment, smoothing logic 2715 is applied to the virtual position and/or virtual positions associated with previously received intraoral scans and/or 2D images. The smoothing operation may reduce or eliminate a jerkiness of the virtual point over time.

In one embodiment, the smoothing logic 2715 is an infinite impulse response (IIR) filter. An IIR filter is a recursive filter in which the output of the filter us computed by using current and previous inputs and previous outputs. In one embodiment, at block 2720 a current virtual position is summed with a previous virtual position delayed at block 2725 and multiplied by a multiplier a₀ at block 2730. The multiplier a₀ may be a scalar value having a value anywhere from 0 to 1 that controls an averaging function. The output of the smoothing logic 2715 is an average position of the virtual point 2735, P̅_{x,y,z}(n).

At block 2740, processing logic determines a velocity of the average position of the virtual point over two or more most recent intraoral scans and/or 2D images. In one embodiment, a previous average position is stored in a delay logic 2745, and summing logic 2750 sums the most recent average position of the virtual point with a negative of the previous average position of the virtual point. Accordingly, a difference between the current position and the previous position of the virtual point is determined. The difference may be divided by the time difference between when the most recent intraoral scan and/or 2D image was generated and when the previous intraoral scan and/or 2D image was generated to determine the velocity of the virtual point associated with the current intraoral scan or 2D image, v(n), where v is the velocity.

In embodiments, the velocity of the point may frequently change as scanning progresses (e.g., as each new intraoral scan and/or 2D image is generated). This may cause the velocity of the virtual position to be jerky. Accordingly, in embodiments a smoothing operation is performed to smooth the velocity of the virtual point over time. In one embodiment, smoothing logic 2760 is applied to the velocity of the virtual position and/or virtual positions associated with previously received intraoral scans and/or 2D images. The smoothing operation may reduce or eliminate a jerkiness of the velocity.

In one embodiment, the smoothing logic 2760 is an infinite impulse response (IIR) filter. In one embodiment, at block 2765 a current velocity is summed with a previous velocity delayed at block 2770 and multiplied by a multiplier a₁ at block 2775. The multiplier a₁ may be a scalar value having a value anywhere from 0 to 1 that controls an averaging function. The output of the smoothing logic 2760 is an average velocity of the virtual point 2780, V_{x,y,z}(n).

At block 2785, processing logic determines a zoom factor for the image or scan n based on the average velocity associated with the image or scan n. In one embodiment, the zoom factor is determined by inputting the average velocity into a function, where the function outputs the zoom factor. The function may be represented as Ψ(v̅_{x,y,z}(n)). In one embodiment, the function is a monotonically decreasing function. In one embodiment, the function that maps velocity to zoom factor is as follows:

$$Ψ ( \overline{v} ) = \frac{M - 1}{1 + α \cdot {\overline{v}}^{2}}$$

Where M is a constant that represents the maximum zoom and a is a constant that represents sensitivity.

In one embodiment, the zoom factor is determined by performing a lookup in a lookup table using the average velocity as a key.

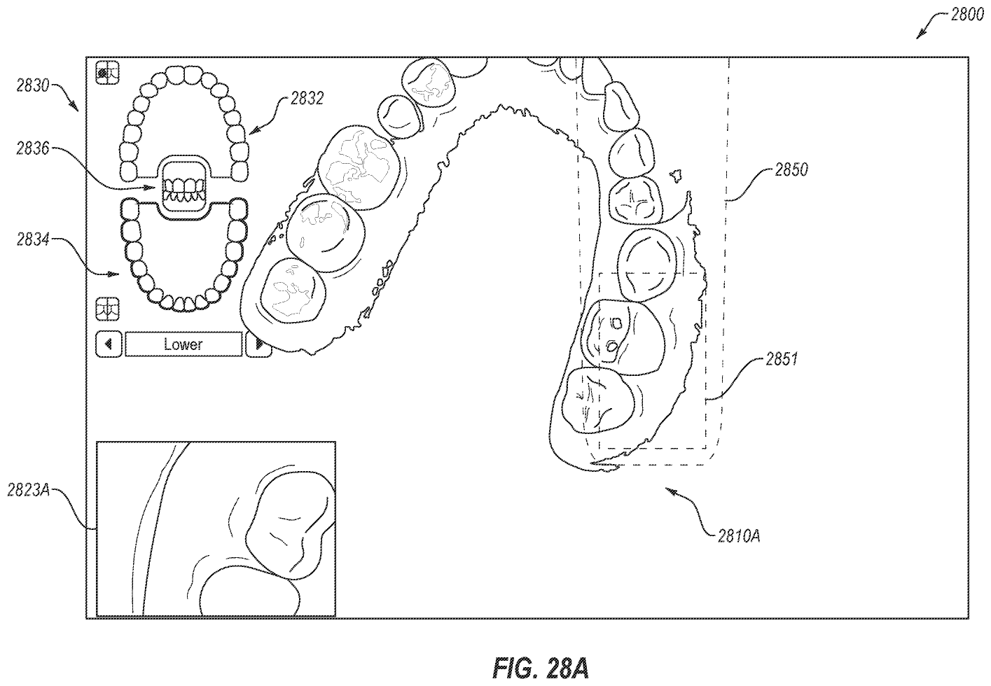

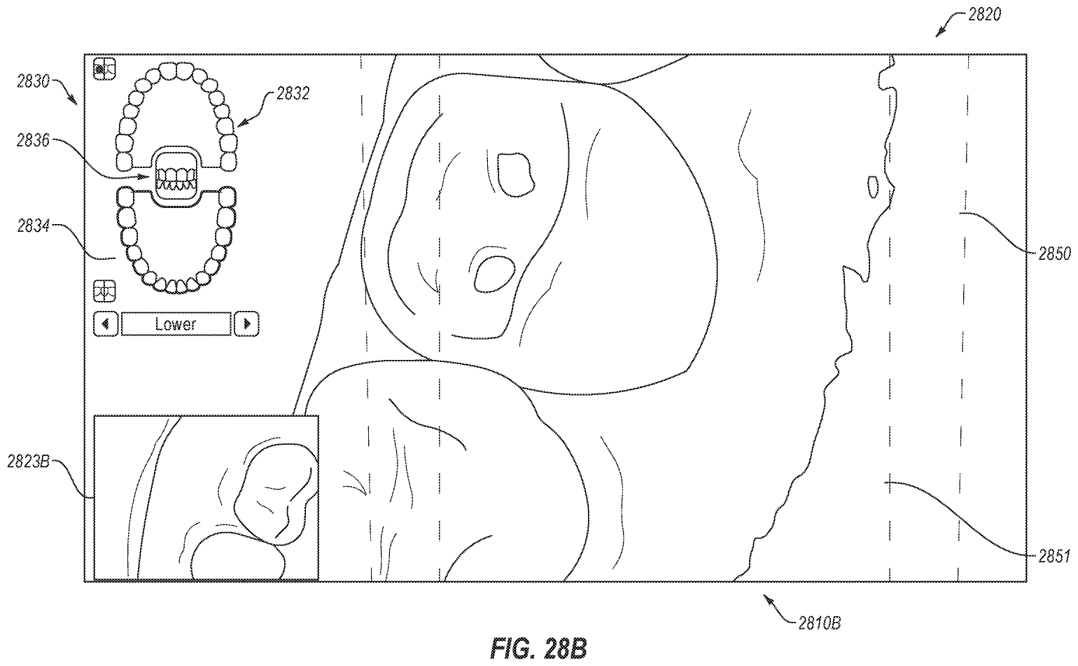

FIG. 28A illustrates a view of a 3D surface 2810A of a dental site with a first zoom setting, in accordance with an embodiment of the present disclosure. FIG. 28A illustrates a view of a graphical user interface 2800 for an intraoral scan application that includes 3D surface 2810A and a 2D image 2823A of a current field of view (FOV) of an intraoral scanner, in accordance with embodiments of the present disclosure. Additionally, an outline 2850 of the intraoral scanner, including an outline 2351 of a field of view of the intraoral scanner may be shown relative to the 3D surface 2810A. The 3D surface 2810A is generated by registering and stitching together multiple intraoral scans captured during an intraoral scanning session. As each new intraoral scan is generated, that scan is registered to the 3D surface and then stitched to the 3D surface. Accordingly, the 3D surface becomes more and more accurate with each intraoral scan, until the 3D surface is complete. A 3D model may then be generated based on the intraoral scans. In one embodiment, as shown, a scan segment indicator 2830 may include an upper dental arch segment indicator 2832, a lower dental arch segment indicator 2834 and a bite segment indicator 2836.

Some regions of a dental arch may be particularly challenging to scan. For example, regions with deep pockets or valleys and distal most molars may be particularly challenging to scan. A user may naturally slow down movement of the intraoral scanner when scanning such regions. In some embodiments, processing logic may compute a velocity of the intraoral scanner (or a virtual point within a field of view of the intraoral scanner) and determine a zoom factor setting based on the velocity. Processing logic may then automatically adjust the zoom factor setting based on the velocity. As shown in FIG. 28A, the scanner velocity may be high, resulting in a relatively low zoom setting (e.g., a zoom setting of 1×).

FIG. 28B illustrates a view of a 3D surface 2810B of a dental site with a second zoom setting, in accordance with an embodiment of the present disclosure. 3D surface 2810B may be an updated version of 3D surface 2810A that has been updated after one or more additional intraoral scans have been stitched to the 3D surface. FIG. 28B illustrates a view of a graphical user interface 2820 for an intraoral scan application that includes 3D surface 2810B and a 2D image 2823B of a current field of view (FOV) of an intraoral scanner, in accordance with embodiments of the present disclosure. Additionally, an outline 2850 of the intraoral scanner, including an outline 2351 of a field of view of the intraoral scanner may be shown relative to the 3D surface 2810B. The 3D surface 2810B is generated by registering and stitching one or more intraoral scans to 3D surface 2810A. Processing logic may have determined that the velocity of the scanner has slowed down relative to the velocity associated with FIG. 28A, and may have automatically adjusted the zoom factor setting based on the updated velocity (e.g., automatically zoomed in on a current region being scanned). As shown in FIG. 28B, the scanner velocity may be low, resulting in a relatively high zoom setting (e.g., a zoom setting of 2×, 3×, 4×, 5×, 6×, 7×, 8×, 9×, 10×, and so on).

FIG. 29 illustrates a diagrammatic representation of a machine in the example form of a computing device 2900 within which a set of instructions, for causing the machine to perform any one or more of the methodologies discussed herein, may be executed. In alternative embodiments, the machine may be connected (e.g., networked) to other machines in a Local Area Network (LAN), an intranet, an extranet, or the Internet. The computing device 2900 may correspond, for example, to computing device 105 and/or computing device 106 of FIG. 1. The machine may operate in the capacity of a server or a client machine in a client-server network environment, or as a peer machine in a peer-to-peer (or distributed) network environment. The machine may be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines (e.g., computers) that individually or jointly execute a set (or multiple sets) of instructions to perform any one or more of the methodologies discussed herein.

The example computing device 2900 includes a processing device 2902, a main memory 2904 (e.g., read-only memory (ROM), flash memory, dynamic random access memory (DRAM) such as synchronous DRAM (SDRAM), etc.), a static memory 2906 (e.g., flash memory, static random access memory (SRAM), etc.), and a secondary memory (e.g., a data storage device 2928), which communicate with each other via a bus 2908.

Processing device 2902 represents one or more general-purpose processors such as a microprocessor, central processing unit, or the like. More particularly, the processing device 2902 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 2902 may also be one or more special-purpose processing devices such as an application specific integrated circuit (ASIC), a field programmable gate array (FPGA), a digital signal processor (DSP), network processor, or the like. Processing device 2902 is configured to execute the processing logic (instructions 2926) for performing operations and steps discussed herein.

The computing device 2900 may further include a network interface device 2922 for communicating with a network 2964. The computing device 2900 also may include a video display unit 2910 (e.g., a liquid crystal display (LCD) or a cathode ray tube (CRT)), an alphanumeric input device 2912 (e.g., a keyboard), a cursor control device 2914 (e.g., a mouse), and a signal generation device 2920 (e.g., a speaker).

The data storage device 2928 may include a machine-readable storage medium (or more specifically a non-transitory computer-readable storage medium) 2924 on which is stored one or more sets of instructions 2926 embodying any one or more of the methodologies or functions described herein, such as instructions for intraoral scan application 115, which may correspond to intraoral scan application 115 of FIG. 1. A non-transitory storage medium refers to a storage medium other than a carrier wave. The instructions 2926 may also reside, completely or at least partially, within the main memory 2904 and/or within the processing device 2902 during execution thereof by the computing device 2900, the main memory 2904 and the processing device 2902 also constituting computer-readable storage media.

The computer readable storage medium 2924 may also store a software library containing methods for the intraoral scan application 115. While the computer-readable storage medium 2924 is shown in an example embodiment to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium other than a carrier wave that is capable of storing or encoding a set of instructions for execution by the machine and that cause the machine to perform any one or more of the methodologies of the present disclosure. The term “computer-readable storage medium” shall accordingly be taken to include, but not be limited to, solid-state memories, and optical and magnetic media.

It is to be understood that the above description is intended to be illustrative, and not restrictive. Many other embodiments will be apparent upon reading and understanding the above description. Although embodiments of the present disclosure have been described with reference to specific example embodiments, it will be recognized that the disclosure is not limited to the embodiments described, but can be practiced with modification and alteration within the spirit and scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

1. An intraoral scanning system, comprising:
an intraoral scanner; and
a computing device, wherein the computing device is to:
receive a plurality of intraoral scans of a dental site from the intraoral scanner during an intraoral scanning session;
generate a three-dimensional (3D) surface of the dental site based on the plurality of intraoral scans;
determine a first surface quality score for a first region of the 3D surface;
output a first view of the 3D surface to a display, wherein the first region of the 3D surface is shown with a first visualization associated with the first surface quality score;
receive one or more additional intraoral scans of the dental site during the intraoral scanning session;
update the 3D surface based on the one or more additional intraoral scans;
determine a new surface quality score for the first region of the updated 3D surface; and
output a first view of the updated 3D surface to the display, wherein the first region of the updated 3D surface is shown with a second visualization associated with the new surface quality score.

2. The intraoral scanning system of claim 1, wherein the computing device is further to:
input at least one of the first region of the 3D surface or the plurality of intraoral scans associated with the first region into a trained machine leaning model, wherein the trained machine learning model outputs the first surface quality score.

3. The intraoral scanning system of claim 1, wherein the plurality of intraoral scans are generated by projecting a structured light comprising a plurality of features onto the dental site and capturing the plurality of features on the dental site, and wherein the first surface quality score is determined based at least in part on a quantity of the plurality of features associated with the first region of the 3D surface.

4. The intraoral scanning system of claim 1, wherein the 3D surface is generated based on a plurality of points from the plurality of intraoral scans, and wherein the computing device is further to:
determine a quality score for each point of the plurality of points;
determine a subset of the plurality of points that are associated with the first region; and
determine the first surface quality score for the first region based on quality scores of the subset of the plurality of points and on a quantity of the subset of the plurality of points.

5. The intraoral scanning system of claim 4, wherein the quality score for a point of the plurality of points is computed based on at least one of:
a) a distance between the point and at least one of a first camera or a second camera of the intraoral scanner that captured the point in generation of an intraoral scan of the plurality of intraoral scans and b) a distance between the first camera and the second camera;
c) a distance between the point and a camera of the intraoral scanner that captured the point in generation of the intraoral scan and d) a distance between the camera and a structured light projector of the intraoral scanner that projected structured light to the point;
a number cameras of the intraoral scanner that captured the point in generation of the intraoral scan;
a spot size associated with the point;
an angle between a normal to the 3D surface at the point and an imaging axis of the camera that captured the point in generation the intraoral scan; or
a type of material of the dental site at the point.

6. The intraoral scanning system of claim 1, further comprising:
determining a first grading rubric for the first region;
determining the first visualization corresponding to the first surface quality score and the second visualization corresponding to the new surface quality score based on the first grading rubric;
determining a third surface quality score for a second region of the 3D surface,
determining a second grading rubric for the second region; and
determining a third visualization corresponding to the third surface quality score based on the second grading rubric, wherein the second region of the 3D surface is shown with the third visualization.

7. The intraoral scanning system of claim 1, wherein the computing device is further to:
determine whether a restorative treatment or an orthodontic treatment is to be performed for the dental site; and
select a grading rubric that associates surface quality scores with visualizations based on whether the restorative treatment or the orthodontic treatment is selected.

8. The intraoral scanning system of claim 1, wherein the computing device is further to:
receive a plurality of two-dimensional (2D) images of the dental site; and
determine a quantity of the plurality of 2D images that depict the first region;
wherein the first surface quality score for the first region is based at least in part on the quantity of the plurality of 2D images that depict the first region.

9. The intraoral scanning system of claim 1, wherein the computing device is further to:
determine a first roughness and a first resolution associated with the first region of the 3D surface;
wherein the first surface quality score is determined based at least in part on the first roughness and the first resolution.

10. The intraoral scanning system of claim 1, wherein the computing device is further to:
determine that the new surface quality score for the first region has failed to improve for a threshold amount of time and that the new surface scanning quality score is below a surface quality threshold;
determine one or more scanning suggestions that, if implemented, would cause the new surface quality score for the first region to improve; and
output the one or more scanning suggestions to the display.

11. The intraoral scanning system of claim 1, wherein the computing device is further to:
determine that the new surface quality score for the first region has failed to improve for a threshold amount of time and that the new surface scanning quality score is below a surface quality threshold; and
increase a zoom setting for the first region.

12. The intraoral scanning system of claim 1, wherein the intraoral scanner comprises a plurality of cameras each having at least one of a different position or a different orientation in the intraoral scanner, and wherein the computing device is further to:
receive a plurality of two-dimensional (2D) images each generated by a different camera of the plurality of cameras;
determine a 2D image of the plurality of 2D images associated with improved intraoral scan quality; and
output the determined 2D image to the display, wherein responsive to output of the determined 2D image, a doctor using the intraoral scanner will reposition the intraoral scanner in a manner that causes the improved intraoral scan quality.

13. The intraoral scanning system of claim 1, wherein the computing device is further to:
determine that the new surface quality score for the first region has failed to improve for a threshold amount of time; and
make a determination that additional intraoral scans of the first region will not improve the updated surface quality score for the region.

14. An intraoral scanning system, comprising:
an intraoral scanner; and
a computing device, wherein the computing device is to:
receive a plurality of intraoral scans of a dental site from the intraoral scanner during an intraoral scanning session;
generate a three-dimensional (3D) surface of the dental site based on the plurality of intraoral scans;
determine a surface quality score for a first region of the 3D surface;
receive one or more additional intraoral scans of the dental site during the intraoral scanning session;
update the 3D surface based on the one or more additional intraoral scans;
update the surface quality score for the first region based on the updated 3D surface;
determine that the surface quality score for the first region fails to satisfy one or more criteria; and
output a notice to at least one of stop generating new intraoral scans of the first region or to move on to a next region.

15. The intraoral scanning system of claim 14, wherein to determine that the surface quality score for the first region fails to satisfy the one or more criteria the computing device is to:
determine that the surface quality score for the first region has failed to improve for a threshold amount of time; and
make a determination that additional intraoral scans of the first region will not improve the surface quality score for the region.

16. The intraoral scanning system of claim 14, wherein to determine that the surface quality score for the first region fails to satisfy the one or more criteria the computing device is to:
determine that the surface quality score for the first region is below a surface quality threshold and that additional surface quality scores of one or more additional regions proximate to the first region are at or above the surface quality threshold.

17. An intraoral scanning system, comprising:
an intraoral scanner; and
a computing device, wherein the computing device is to:
receive a plurality of intraoral scans of a dental site from an intraoral scanner during an intraoral scanning session;
determine a quality score for each point of a plurality of points in one or more of the plurality of intraoral scans;
generate a three-dimensional (3D) surface based on the plurality of points from the one or more of the plurality of intraoral scans;
determine a first surface quality score for a first region of the 3D surface based at least in part on a) a quantity of points, from the plurality of points, that are associated with the first region and b) quality scores of the points that are associated with the first region; and
output a view of the 3D surface to a display, wherein the first region of the 3D surface is shown with a first visualization associated with the first surface quality score.

18. The intraoral scanning system of claim 17, wherein the computing device is further to:
input data for the plurality of points from the one or more of the plurality of intraoral scans into a trained machine learning model, wherein the trained machine learning model outputs the quality score for each of the plurality of points.

19. The intraoral scanning system of claim 17, wherein the computing device is further to:
determine a first roughness and a first resolution associated with the first region of the 3D surface;
wherein the first surface quality score is determined based at least in part on the first roughness and the first resolution.

20. The intraoral scanning system of claim 19, wherein:
the first resolution is determined based at least in part on the quantity of points that are associated with the first region; and
the first roughness is determined based on distances between the points associated with the first region and nearest points on the 3D surface.
